(19) United States
(12) Patent Application Publication
(10) Pub. No.: US 2015/0290235 A1
(43) Pub. Date: Oct. 15, 2015

(54) USE OF SMALL MOLECULE INHIBITORS/ACTIVATORS IN COMBINATION WITH (DEOXY)NUCLEOSIDE OR (DEOXY)NUCLEOTIDE ANALOGS FOR TREATMENT OF CANCER AND HEMATOLOGICAL MALIGNANCIES OR VIRAL INFECTIONS

(71) Applicant: AB SCIENCE, Paris (FR)

(72) Inventors: Laurent Gros, Marseille (FR); Patrice Dubreuil, Marseille (FR); Alain Moussy, Paris (FR); Stephane Audebert, Marseille (FR); Colin Mansfield, Ecully (FR)

(21) Appl. No.: 14/646,775

(22) PCT Filed: Nov. 8, 2013
(86) PCT No.: PCT/EP2013/073442

§ 371 Date: May 22, 2015

Related U.S. Application Data
(60) Provisional application No. 61/729,453, filed on Nov. 23, 2012.

(51) Int. Cl. A61K 31/7068; A61K 31/496

(52) U.S. Cl. CPC A61K 31/7068; A61K 31/496

(57) ABSTRACT

A method for treating patients afflicted with cancer (including hematological malignancies) or viral infections, wherein the patients are under treatment or are to be treated with at least one anticancer or antiviral agent, and in particular (deoxy)nucleotide or (deoxy)nucleoside analog drugs, includes administering at least one small molecule inhibitor/activator (including ATP competitive inhibitors, signal transduction inhibitors/activators, protein kinase inhibitors/activators, and tyrosine kinase inhibitors/activators) in combination with the (deoxy) nucleotide or (deoxy)nucleoside analog, and wherein the small molecule inhibitor/activator is administered in sufficient amount to modulate deoxynucleotide or deoxynucleoside kinase activity (and in particular deoxycytidine kinase activity) to modulate activation of the (deoxy)nucleotide or (deoxy)nucleoside analog in vivo with a subsequent therapeutically beneficial anticancer or antiviral effect. The combined treatments together include a therapeutically effective amount.

The present invention relates to a method for treating patients afflicted with cancer (including hematological malignancies) or viral infections, wherein said patients are under treatment or are to be treated with at least one anticancer or antiviral agent, and in particular (deoxy)nucleotide or (deoxy)nucleoside analog drugs, comprising administering at least one small molecule inhibitor/activator (including ATP competitive inhibitors, signal transduction inhibitors/activators, protein kinase inhibitors/activators, and tyrosine kinase inhibitors/activators) in combination with said (deoxy)nucleotide or (deoxy)nucleoside analog, and wherein said small molecule inhibitor/activator is administered in sufficient amount to modulate deoxynucleotide or deoxynucleoside kinase activity (and in particular deoxycytidine kinase activity) to modulate activation of said (deoxy)nucleotide or (deoxy)nucleoside analog in vivo with a subsequent therapeutically beneficial anticancer or antiviral effect. The combined treatments together comprise a therapeutically effective amount.

### BACKGROUND OF THE INVENTION

### Overview of Small Molecule Inhibitors/Activators

A small molecule drug is a compound with medicinal properties, characteristically with a molecular weight of less than 1000 Daltons, and typically between 300 and 700 Daltons. The advantages offered by small molecule drugs is their ability to enter into parts of the body that larger molecules cannot, for example, penetrating directly into cells, and that they are often orally bioavailable. Although small molecule drugs are frequently developed for their properties to act as enzyme inhibitors, i.e. a molecule that binds to an enzyme to decrease its activity, they also offer the ability of activating enzymes, i.e. a molecule that binds to an enzyme to increase its enzymatic activity. Such small molecule activators typically achieve this by either removing factors that inhibit activity or by producing changes to the enzyme to foster catalytic activity. In certain cases these small molecule drugs can serve as duel inhibitor/activator; for example, the activation of a given kinase serving as an effector mechanism to inhibit a targeted signaling pathway. Subcategories of small molecule inhibitors/activators include ATP competitive inhibitors, signal transduction inhibitors/activators, protein kinase inhibitors/activators, and tyrosine kinase inhibitors/activators. Protein kinases regulate the majority of cellular pathways, especially those involved in signal transduction by catalyzing phosphorylation reactions. Phosphorylation consists of delivering a single phosphoryl group from the adenosine triphosphate (ATP) to protein substrates. Phosphorylation usually results in a functional change of the substrate by shifting enzyme activity, cellular location, or association with other proteins. More than 500 protein kinases are predicted to exist, based on the human genome sequencing, which are grouped into three main classes based upon substrate preferences: serine-threonine kinases, tyrosine kinases, and so called dual-function kinases (i.e. both serine-threonine and tyrosine kinases).

Normally, protein kinase activity is strictly regulated, however, under pathological conditions protein kinases can be deregulated, leading to alterations in the phosphorylation and resulting in uncontrolled cell division, inhibition of apoptosis, and other disease causing abnormalities. Such aberrations in cell signaling pathways are the cause of many human and animal proliferative diseases and many human inflammatory diseases. For example, tyrosine kinases play a fundamental role in signal transduction and deregulated activity of these enzymes has been observed in cancer, benign proliferative disorders, and inflammatory diseases. Tyrosine kinases are found on the cell surface (receptor tyrosine kinases) and also in the cytoplasm and nucleus of cells, where they participate in signal transduction and regulation of gene transcription. In the normal cell, a growth factor can bind to its tyrosine kinases receptor, which then becomes activated and passes on the signal internally via binding ATP and then adding phosphate groups to itself (autophosphorylation) and to other molecules further down the pathway. At least 20 types of proteins that can be found on the cell surface are included in the family of receptor tyrosine kinases. Examples include c-Kit, epidermal growth factor receptor (EGFR), vascular endothelial growth factor receptor (VEGFR), and platelet-derived growth factor receptor (PDGFR).

While protein kinase signaling is critical for normal development and life processes, unregulated signaling can lead to uncontrolled cell growth and survival and thus is one of the underlying causes of some types of cancer. Small molecule inhibitors/activators (including ATP competitive inhibitors, signal transduction inhibitors/activators, protein kinase inhibitors/activators, and tyrosine kinase inhibitors/activators) are a way to more directly target a cancer cell compared with traditional cytotoxic drugs. Small molecule inhibitors/activators have been approved for treatment of certain types of cancer in humans and dogs. Examples of small molecule inhibitors/activators that have been approved for cancer treatment are shown in Tables 1 and 2. Many other small molecule inhibitors/activators are under development. Examples include, but are not limited to: afatinib, alitretinoin, axitinib, bafetinib, bexarotene, BI-2536, bosutinib, brivanib, canertinib, cediranib, CP724714, crizotinib, dasatinib, danusertib, dovitinib, E7080, erlotinib, everolimus, fostamatinib, gefitinib, imatinib, lapatinib, lestaurtinib, linsitinib, masitinib, motesanib, neratinib, nilotinib, NVP TAE-684, OSI-027, OSI-420, OSI-930, pazopanib, pelitinib, PF573228, regorafenib, romidepsin, ruxolitinib, saracatinib, sorafenib, sunitinib, TAE226, TAE684, tandutinib, telatinib, tautinib, temsirolimus, toceranib, tofacitinib, tozasertib, tretinoin, vandetanib, vatalanib, vemurafenib, vorinostat and WZ 4002.

One of the most effective approaches to modify signaling associated with protein kinases or tyrosine kinases has been to use small molecules that block the ATP binding site of the kinase. With this blockage, small molecule inhibitors, also referred to as ATP competitive inhibitors, protein kinase inhibitors, and tyrosine kinase inhibitors depending upon their specific targets or mechanisms of action, prevent the kinase from phosphorylating and beginning the signaling cascade, which can lead to an inhibitory/fatal effect on cells reliant upon the kinase signaling pathway being inhibited, or “downstream” consequences of this; for example, impeding new blood vessel growth (angiogenesis).

### Overview of (Deoxy)Nucleotide and (Deoxy)Nucleoside Analog Drugs

(Deoxy)nucleotide and (deoxy)nucleoside analogs are synthetic molecules that resemble a naturally occurring nucleotide or nucleoside, but that lack a bond site needed to link it to an adjacent nucleotide or nucleoside. These drugs can act as inhibitors of viral and cellular replication. They are among the most important therapeutic agents currently used to treat tumors and viral diseases. Cytotoxic (deoxy)nucleoside analogs such as capecitabine (Xeloda®), cladribine (Litak®), cytarabine (Cytosar-U®), decitabine (Dacogen®), fluorouracil (5FU, Adrucil®), fludarabine (Fludara®), and gemcitabine (Gemzar®) are commonly used in chemotherapy of cancer. Other (deoxy)nucleoside analogs, such as zidovudine (Retrovir®), lamivudine (Epivir®), and abacavir (Ziagen®), or (deoxy)nucleotide analogs such as tenofovir (Viread®), are used in treatment of viral infections such as human immunodeficiency virus (HIV) infection.

(Deoxy)nucleotide and (deoxy)nucleoside analogs (also referred to as nucleotide analog reverse-transcriptase inhibitors [NtARTIs or NtRTIs] and nucleoside analog reverse-transcriptase inhibitors [NARTIs or NRTIs]) are classified as competitive substrate inhibitors. That is to say, they are analogs of the naturally occurring deoxynucleotides or deoxynucleosides needed to synthesize the viral DNA or RNA, respectively, which will compete with the natural deoxynucleotides/deoxynucleosides for incorporation into the growing viral DNA/RNA chain. (Deoxy)nucleotide and (deoxy)nucleoside analog drugs have various modes of action, however, a common feature for most (deoxy)nucleotide and (deoxy)nucleoside analogs is a process called chain termination. Many of these drugs require a phosphorylation by nucleoside and nucleotide kinases to become pharmacologically active, i.e. monophosphylated, biphosphylated or triphosphylated. The phosphorylated (deoxy)nucleotide or (deoxy)nucleoside analogs then disrupt the normal functions of DNA or RNA leading to cell death or inhibition of viral replication. In general, for antiviral treatment, analogs of (deoxy)nucleotides or (deoxy)nucleosides needed to synthesize the viral DNA/RNA, compete with their natural substrate counterpart for incorporation into the growing viral DNA/RNA chain. However, structural differences designed into the analog prevent bonding of subsequent (deoxy)nucleotides or (deoxy)nucleosides thus, stopping viral DNA/RNA synthesis. Likewise, for anticancer treatment, analogs of (deoxy)nucleotides or (deoxy)nucleosides compete with their natural substrate counterpart for incorporation into DNA/RNA; however, structural differences designed into the analog interfere with DNA/RNA production and therefore normal cell development and division. In this manner, inhibition of cell division harms tumor cells more than other cells because the proliferation rate of cancer cells is greater than other cells.

Overview of Deoxycytidine Kinase (dCK)

Many (deoxy)nucleotide and (deoxy)nucleoside analogs need to be phosphorylated to a monophosphate, diphosphate, or triphosphate form intracellularly for a complete pharmacological activity. For example, certain (deoxy)nucleotide and (deoxy)nucleoside analogs, including the commonly used analog drugs of cytarabine (Ara-C) and gemcitabine, are phosphorylated to a triphosphate form before incorporation into DNA/RNA. One possible mode of action of (deoxy)nucleotide and (deoxy)nucleoside analogs is through inhibition of DNA/RNA synthesis after incorporation of its phosphorylated form into the replicating DNA/RNA strand. This phosphorylation step typically involves deoxynucleoside or deoxynucleotide kinases; for example, phosphorylation is mainly catalyzed by the deoxynucleoside kinase known as deoxycytidine kinase (dCK). Deoxycytidine kinase is also involved in the activation of certain demethylating agents, for example the DNA methyltransferase inhibitor decitabine (5-aza-29-deoxycytidine). Once inside the cell decitabine undergoes three steps of phosphorylation to achieve its active form, with the initial rate-limiting monophosphorylation being controlled by the deoxycytidine kinase.

Human deoxycytidine kinase (hdCK) is an essential deoxynucleoside kinase implied in the biosynthesis of the nucleotide precursors used for cellular DNA synthesis. Among nucleotide kinases, dCK has the unique property to use either ATP or UTP as a phosphate donor, although several enzymatic and structural studies have established that UTP is the true physiological hDCK-phosphate donor [Hughes T L, et al. 1997 Biochemistry 36(24): 7540; Godsey M H, et al. 2006 Biochemistry 45(2): 452]. hDCK is required for the phosphorylation of several deoxyribonucleosides and their nucleoside analogs: 2′-deoxy-adenosine (2′dA), 2′-deoxy-guanosine (2′dG) et 2′-deoxy-cytosine (2′dC). hDCK is equally responsible for the activation by phosphorylation of a number of nucleoside-like prodrugs widely used in the anticancer and/or antiviral chemotherapy such as 2′-Deoxy-2′,2′-difluorocytidine (gemcitabine), 1-(β-D-Arabino-furanosyl)-cytosine (ARAC), 2-Chloro-2′-deoxyadenosine (2CdA, cladribine), 9-β-D-Arabinofuranosyl-2-fluoroadenine (F-ARA-A/fludarabine), 2′,3′-Dideoxy-3′-thiacytidine (L-3TC/lamivudine) or 5-Aza-2′-deoxycytidine (decitabine). Thus, dCK plays an important role in activation of (deoxy)nucleotide and (deoxy)nucleoside analogs.

### Current Limitations of (Deoxy)Nucleotide and (Deoxy)Nucleoside Analog Drugs

The clinical use of (deoxy)nucleotide and (deoxy)nucleoside analogs is often limited by high toxicity in healthy tissues or resistance mechanisms that reduce the patient's susceptibility and therefore the drug's potency. Despite advances in the development of (deoxy)nucleotide and (deoxy)nucleoside analogs and their use in combination therapies, most patients either do not achieve remission or relapse after an initial therapeutic response.

As might be expected of drugs such as (deoxy)nucleotide and (deoxy)nucleoside analogs that interfere with DNA/RNA synthesis, there are significant adverse effects with any organs or processes that rely on cell division, such as the replenishment of red and white blood cells. These drugs can also interfere with the energy regulating organelles known as mitochondria because they have their own DNA, without the protective mechanisms of the cell nucleus. The toxicity is classified according to the structure and chemical properties of the specific analog. General symptoms of (deoxy)nucleotide and (deoxy)nucleoside analog toxicity include peripheral neuropathy, myopathy, bone marrow suppression and pancreatitis. This toxicity can either be acute but sometimes also be delayed and occur after several weeks or months of drug treatment. Effectiveness and toxicity of any given nucleoside analog depend on several factors including uptake, transport, metabolic activation, incorporation and degradation. Mitochondrial toxicity is a severe side effect of several clinically used (deoxy)nucleotide and (deoxy)nucleoside analogs, especially for combination regimens, with complications including fatal hepatic failure, peripheral neuropathy, pancreatitis, and symptomatic hyperlactatemia/lactic acidosis.

Development of drug resistance is another major problem in the treatment of cancers and viral infection. Resistance can be either inherent or acquired. Inherent resistance is a quality of several tumor types, which is reflected in low response rates in clinical trials. Acquired resistance can develop by selection of cells with drug resistance mutations from a heterogeneous tumor cell population during repetitive treatment with a drug.

### AIMS OF THE INVENTION

There is an urgent need to discover suitable methods for the treatment of cancer (including hematological malignancies) or viral disease, including combination treatments that result in decreased side effects and that are effective at treating and controlling cancers or viral infection.

The invention aims to solve the technical problem of providing an active ingredient that improves prior art methods for the treatment of cancer (including hematological malignancies) or viral disease, in human patients receiving treatment in either first line or second line and beyond, where said active ingredient is administered in combination with at least one anticancer or antiviral therapeutic agent.

The invention also aims to solve the technical problem of providing an active ingredient that improves prior art methods for the treatment of cancer (including hematological malignancies) or viral disease, in human patients receiving treatment in either first line or second line and beyond, where said active ingredient is administered in combination with at least one (deoxy)nucleotide or (deoxy)nucleoside analog.

The invention also aims to solve the technical problem of providing an active ingredient that when administered in combination with at least one anticancer or antiviral therapeutic agent increases the amount of said anticancer or antiviral therapeutic agent's active ingredient available for cellular uptake and/or the increased intracellular concentration of said anticancer or antiviral therapeutic agent's active ingredient.

In one embodiment, the invention aims to solve the technical problem of providing an active ingredient that produces a therapeutically beneficial effect when administered in combination with at least one anticancer or antiviral therapeutic agent, especially (deoxy)nucleotide or (deoxy)nucleoside analog drugs, with the advantage of decreasing the dose of the aforementioned anticancer or antiviral therapeutic agent(s) with subsequent decrease in unwanted or harmful side effects, whilst simultaneously maintaining a therapeutically effective amount of the aforementioned anticancer or antiviral therapeutic agent(s). This is sometimes referred to as a ‘dose-sparing’ strategy, in this case with respect to the (deoxy)nucleotide or (deoxy)nucleoside analog drugs, i.e. an analogy-sparing strategy.

In another embodiment, the invention aims to solve the technical problem of providing an active ingredient that produces a therapeutically beneficial effect when administered in combination with at least one anticancer or antiviral therapeutic agent, especially (deoxy)nucleotide or (deoxy)nucleoside analog drugs, for the treatment of cancer (including hematological malignancies) or viral disease in a human patient, wherein said patient is refractory or resistant to said anticancer or antiviral therapeutic agent(s).

In yet another embodiment, the invention aims to solve the technical problem of providing an active ingredient that when administered in combination with at least one other anticancer or antiviral therapeutic agent, especially (deoxy)nucleotide or (deoxy) nucleoside analog drugs, promotes an extended treatment period for the aforementioned anticancer or antiviral therapeutic agent(s) by retarding the onset of acquired drug resistance; i.e. it acts as maintenance therapy.

The invention aims to provide an efficient treatment for such diseases at an appropriate dose, route of administration and daily intake.

### SUMMARY OF THE INVENTION

Deoxycytidine kinase (dCK) is required for the phosphorylation of several antiviral and anticancer (deoxy)nucleotide and (deoxy)nucleoside analogs drugs, with lack of response or resistance to these agents possibly being associated with a loss or decrease in dCK activity.

Strategies aiming to enhance the therapeutic effects of (deoxy)nucleotide or (deoxy)nucleoside analog drugs, for example, through stimulation of dCK activity, could be a great benefit to patients suffering from cancer (including hematological malignancies) or viral infections. Thus, one possible solution is the development of (deoxy)nucleotide and (deoxy)nucleoside analog-sensitizing agent. In the absence of drug resistance, such a sensitizing agent would permit lower doses of the (deoxy)nucleotide and (deoxy)nucleoside analogs to be administered for equivalent potency compared with the standard higher dosage leading to lower toxicity, improved treatment compliance and long-term administration. Alternatively, drugs capable of overcoming an under-expression, down-regulation, or decreased activity of dCK may be useful in counteracting inherent and acquired resistance, thereby facilitating the prolonged therapeutic benefits of (deoxy)nucleotide and (deoxy)nucleoside analogs.

The invention relates to the discovery that at least one small molecule inhibitor/activator (including ATP competitive inhibitors, signal transduction inhibitors/activators, protein kinase inhibitors/activators, and tyrosine kinase inhibitors/activators) and in particular masitinib or a pharmaceutically acceptable salt or hydrate thereof, can be used in combination with one or more anticancer or antiviral agents, especially (deoxy)nucleotide or (deoxy)nucleoside analog drugs, to provide therapeutically beneficial anticancer or antiviral effects.

The present invention relates to a method for treating patients afflicted with cancer (including hematological malignancies) or viral infections, wherein said patients are under treatment or are to be treated with at least one anticancer or antiviral agent, and in particular (deoxy)nucleotide or (deoxy)nucleoside analog drugs, comprising administering at least one small molecule inhibitor/activator (including ATP competitive inhibitors, signal transduction inhibitors/activators, protein kinase inhibitors/activators, and tyrosine kinase inhibitors/activators) in combination with said (deoxy)nucleotide or (deoxy)nucleoside analog, and wherein said small molecule inhibitor/activator is administered in sufficient amount to modulate (deoxy)nucleotide or (deoxy)nucleoside kinase activity (and in particular deoxycytidine kinase activity), notably to modulate activation of said (deoxy)nucleotide or (deoxy)nucleoside analog in vivo with a subsequent therapeutically beneficial anticancer or antiviral effect. The combined treatments together comprise a therapeutically effective amount.

The invention relates to a method for the treatment of a cancer (including hematological malignancies) or a viral infection in a human patient, wherein said method comprises administering to a human patient at least one small molecule inhibitor/activator in combination with at least one anticancer or antiviral drug.

In one embodiment the invention also relates to the treatment of patients afflicted with cancer (including hematological malignancies) or viral infection, wherein said patients are under treatment or are to be treated with one or more anticancer or antiviral agents, especially (deoxy)nucleotide or (deoxy)nucleoside analog agents, comprising administering at least one small molecule inhibitor/activator (including ATP competitive inhibitors, signal transduction inhibitors/activators, protein kinase inhibitors/activators, and tyrosine kinase inhibitors/activators) in combination with at least one anticancer or antiviral agent, and wherein said small molecule inhibitor(s) are administered in sufficient amount to modulate deoxynucleotide or deoxynucleoside kinase activity, and in particular deoxycytidine kinase activity, with a subsequent increased bioavailability (increased amount of said anticancer or antiviral therapeutic agent's active ingredient being available for cellular uptake and/or the increased intracellular concentration of said anticancer or antiviral therapeutic agent's active ingredient) and/or with a subsequent increased phosphorylation of said anticancer or antiviral drug(s).

In another embodiment, the invention relates to the treatment of patients afflicted with cancer (including hematological malignancies) or viral infection, wherein said patients are under treatment or are to be treated with one or more anticancer or antiviral agents, comprising administering at least one small molecule inhibitors/activator (including ATP competitive inhibitors, signal transduction inhibitors/activators, protein kinase inhibitors/activators, and tyrosine kinase inhibitors/activators) in combination with at least one (deoxy)nucleotide or (deoxy)nucleoside analog agents, and wherein said small molecule inhibitor(s) are administered in sufficient amount to modulate deoxynucleotide or deoxynucleoside kinase activity, and in particular deoxycytidine kinase activity, to modulate phosphorylation of said (deoxy)nucleotide or (deoxy)nucleoside analog in vivo.

In another embodiment, the invention relates to the treatment of patients afflicted with cancer (including hematological malignancies) or viral infection, in which at least one small molecule inhibitors/activator (including ATP competitive inhibitors, signal transduction inhibitors/activators, protein kinase inhibitors/activators, and tyrosine kinase inhibitors/activators) and at least one anticancer or antiviral agent, especially (deoxy)nucleotide or (deoxy)nucleoside analog agents, are administered to patients in need thereof, and wherein said small molecule inhibitor(s)/activator(s), inhibits the activity of one or more protein kinases, including and without particular limitation: c-Kit, Lyn, Fyn, Lck and other Src family kinases, platelet-derived growth factor receptor (PDGFR), Fms, Flt3, Abelson proto-oncogene (ABL), anaplastic lymphoma kinase (AKL), epidermal growth factor receptor (EGFR), fibroblast growth factor receptor (FGFR), Human EGFR type 2 (HER2), hepatocyte growth factor receptor (HGFR/Met), Ron, Mer, Axl, insulin-like growth factor-1 receptor (IGF-1R), JAK, FAK, PLK, Aurora kinases, Pim kinases or vascular endothelial growth factor receptor (VEGFR).

In another embodiment, the invention relates to the treatment of patients afflicted with cancer, wherein said patients are under treatment or are to be treated with at least one anticancer agent, especially (deoxy)nucleotide or (deoxy)nucleoside analog agents, and who are not refractory or resistant to said anticancer agent(s), wherein at least one small molecule inhibitors/activator (including ATP competitive inhibitors, signal transduction inhibitors/activators, protein kinase inhibitors/activators, and tyrosine kinase inhibitors/activators) and in particular masitinib or a pharmaceutically acceptable salt or hydrate thereof, is administered in combination with said anticancer agent(s), and wherein said small molecule inhibitor(s) produces a dose-sparing effect on the anticancer agent(s).

In yet another embodiment of this invention, at least one small molecule inhibitors/activator (including ATP competitive inhibitors, signal transduction inhibitors/activators, protein kinase inhibitors/activators, and tyrosine kinase inhibitors/activators) and in particular masitinib or a pharmaceutically acceptable salt or hydrate thereof, is administered in combination with at least one anticancer agent, especially (deoxy)nucleotide or (deoxy)nucleoside analog drugs, for the treatment of patients afflicted with cancer, wherein said patients are refractory or resistant to said anticancer agent(s).

In another embodiment, the invention relates to the treatment of patients afflicted with viral infection, wherein said patients are under treatment or are to be treated with at least one anticancer agent, especially (deoxy)nucleotide or (deoxy)nucleoside analog agents, and who are not refractory or resistant to said antiviral agent(s), wherein at least one small molecule inhibitors/activator (including ATP competitive inhibitors, signal transduction inhibitors/activators, protein kinase inhibitors/activators, and tyrosine kinase inhibitors/activators) and in particular masitinib or a pharmaceutically acceptable salt or hydrate thereof, is administered in combination with said anticancer agent(s), and wherein said small molecule inhibitor(s) produces a dose-sparing effect on the antiviral agent(s).

In yet another embodiment of this invention, at least one small molecule inhibitors/activator (including ATP competitive inhibitors, signal transduction inhibitors/activators, protein kinase inhibitors/activators, and tyrosine kinase inhibitors/activators) and in particular masitinib or a pharmaceutically acceptable salt or hydrate thereof, is administered in combination with at least one antiviral agent, especially (deoxy)nucleotide or (deoxy)nucleoside analog drugs, for the treatment of patients afflicted with viral infection, wherein said patients are refractory or resistant to said antiviral agent(s).

In another embodiment, the invention relates to the treatment of a cancer in a human patient, wherein said method comprises administering to a human patient at least one tyrosine kinase inhibitor optionally in combination with at least one anticancer drug, wherein said patient is selected from patients naïve to at least one anticancer drug, or responding to treatment with said at least one anticancer drug; patients resistant, intolerant, or refractory to said at least one anticancer drug, and patients with an under-expression, down-regulation, or decreased activity of dCK.

In another embodiment, the invention relates to the treatment of a viral infection in a human patient, wherein said method comprises administering to a human patient at least one tyrosine kinase inhibitor optionally in combination with at least one antiviral drug, wherein said patient is selected from patients naïve to at least one antiviral drug, or responding to treatment with said at least one antiviral drug; patients resistant, intolerant, or refractory to said at least one antiviral drug, and patients with an under-expression, down-regulation, or decreased activity of dCK.

### DESCRIPTION OF THE INVENTION

Many (deoxy)nucleotide and (deoxy)nucleoside analogs need to be phosphorylated to a monophosphate, diphosphate, or triphosphate form, for pharmacological activity. Phosphorylation is typically catalyzed by deoxynucleoside or deoxynucleotide kinases, for example, deoxycytidine kinase (dCK). The initial phosphorylation of the (deoxy)nucleotide or (deoxy)nucleoside analog to its monophosphate form is often the rate-limiting step in the activation process. Thus, accumulation of the analog drug is higher in cells that contain high levels of activating enzymes. For this reason, phosphorylation catalyzed by the deoxynucleoside kinase dCK plays a pivotal role in activation of numerous (deoxy)nucleotide and (deoxy)nucleoside analogs, including gemcitabine, cytarabine (Ara-C), and cladribine (2-CdA). The deoxycytidine kinase is also important in the activation of certain demethylating agents, for example the DNA methyltransferase inhibitor decitabine (5-aza-2-deoxycytidine). Once inside the cell decitabine undergoes three steps of phosphorylation to achieve its active form, with the initial rate-limiting monophosphorylation being orchestrated by deoxycytidine kinase.

In one example mode of action, deoxynucleoside kinases are enzymes that catalyze the chemical reaction:

<<ATP/UTP+2′-deoxynucleoside # ADP/UDP+2′-deoxynucleoside 5′-phosphate>>

The two substrates of this enzyme are ATP/UTP and 2′-deoxynucleoside, whereas its two products are ADP/UDP and 2′-deoxynucleoside 5′-phosphate.

In the mode of action shown below, it is illustrated how the deoxycytidine kinase is essential for phosphorylation of gemcitabine (2′,2′-difluorodeoxycytidine), a deoxycytidine antimetabolites drug active against various solid tumors.

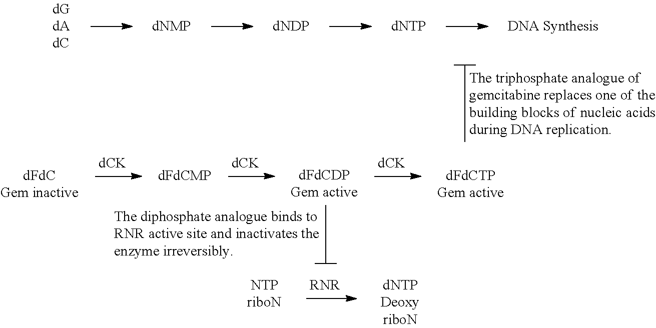

Gemcitabine is a structural analog (difluoro form) of deoxycytidine nucleoside, which inhibits DNA synthesis both in direct competition with dCTP [d(eoxy)-+c(ytidine)+t(ri)p(hosphate)] under its dFdC 5′-triphosphate (dFdCTP) form, and indirectly at the level of the deoxyribonucleotides synthesis by blocking irreversibly the RiboNucleotides Reductase (RNR) activity through its dFdCDP form.

A similar activation process is used for all the nucleotides analogs via nucleotide kinases, especially deoxycytidine kinase (dCK).

The problem of resistance to (deoxy)nucleotide and (deoxy)nucleoside analogs has been well investigated for the nucleoside analog gemcitabine (Gemzar®, Eli Lilly and Company), an analog of deoxycytidine with activity against several solid tumors. Gemcitabine enters the cell via a facilitated nucleoside transport mechanism and is phosphorylated into gemcitabine 5′-monophosphate (dFd-CMP) by deoxycytidine kinase (dCK). It is then subsequently phosphorylated by other pyrimidine kinases to the active 5′-diphosphate (dFd-CDP) and triphosphate (dFd-CTP) derivatives. In association with dCK's role in activation of (deoxy)nucleotide or (deoxy)nucleoside analog drugs, several researchers have linked abnormal dCK activity with acquired resistance to gemcitabine in cell and animal models [Bergman A M, et al. Drug Resistance Updates 2002, 5:19; Ruiz van Haperen V W, et al. Cancer Res 1994, 54:4138; Dumontet C, et al. Br J Haematol 1999, 106:78; van der Wilt C L, et al. Adv Exp Med Biol 2000, 486:287]. In one study by Galmarini et al. [BMC Pharmacology 2004, 4:8], analysis of the mechanisms of resistance in gemcitabine-resistant tumor cells via in vitro models and mouse xenografts suggested that partial deletion of the dCK gene was involved with resistance to gemcitabine. Cytarabine (Ara-C, Cytosar-U®) is another analog of deoxycytidine that has been studied in relation to the problem of resistance. This drug is effective in the treatment of different forms of leukemia. Again, under-expression, down-regulation, or decreased activity of dCK has been associated with resistance to cytarabine in various resistant cell lines [Verhoef V, et al. Cancer Res 1981, 41:4478; Bhalla K, et al. Cancer Res 1984, 44:5029; Stegmann A P, et al. Leukemia 1993, 7:1005]. Indeed, transfection of the dCK gene in dCK-deficient tumor cell lines has been shown to restore in vitro sensitivity to cytarabine [Stegmann A P, et al. Blood 1995, 85:1188; Hapke D M, et al. Cancer Res 1996, 56:2343]. Furthermore, in vitro models have shown cross-resistance between Cladribine (Litak®), gemcitabine, fludarabine (Fludara®) and cytarabine with reduced dCK activity as the underlying determinant of resistance [Dumontet C, et al. Br J Haematol 1999, 106:78; Orr R M, et al. Clin Cancer Res 1995; 1:391]. Cross-resistance is a resistance to a particular drug that often results in resistance to other drugs from a similar chemical class, to which the cells may not have been exposed.

However, there are many other possible resistance mechanisms against (deoxy)nucleotide and (deoxy)nucleoside analogs such as gemcitabine. Bergman et al. summarized these as including: an increased activity of dCDA; an increased ribonucleotide reductase activity; a decreased accumulation of triphosphates; or an altered DNA polymerase [Bergman A M, et al. Drug Resistance Updates 2002, 5:19]. Galmarini et al. described three main mechanisms of resistance: (1) a primary mechanism of resistance to (deoxy)nucleotide and (deoxy)nucleoside analogs arise from an insufficient intracellular concentration of (deoxy)nucleotide and (deoxy)nucleoside analog triphosphates, which may result from inefficient cellular uptake, reduced levels of activating enzymes, increased (deoxy)nucleotide and (deoxy)nucleoside analog degradation, or expansion of the deoxyribonucleotide triphosphate pools; (2) an inability to achieve sufficient alterations in DNA strands or deoxyribonucleotide triphosphate pools, either by altered interaction with DNA polymerases, by lack of inhibition of ribonucleotide reductase, or because of inadequate p53 exonuclease activity; and (3) drug resistance by consequence of a defective induction of apoptosis.

Hence, under-expression, down-regulation, or decreased activity of dCK would appear to be only one possible mechanism of resistance to gemcitabine, and therefore of (deoxy)nucleotide and (deoxy)nucleoside analogs in general. Furthermore, this link is itself controversial with proof being mostly restricted to in vitro experimentation, typically with resistance established using continuous exposure to gemcitabine at increasing concentrations, which appears difficult to reproduce under in vivo conditions and are therefore of limited clinical relevance. Indeed, a study by Bergman et al. that developed the first model with in vivo induced resistance to gemcitabine, those resistance mechanisms known from in vitro studies (e.g. dCK, dCDA, and DNA polymerase) did not reveal a clear explanation, and concluded that dCK activity was not the most important determinant of gemcitabine resistance. In contrast to many in vitro findings, this study identified increased expression of ribonucleotide reductase subunit M1 (RRM1) as the major determinant of acquired gemcitabine resistance in vivo [Bergman et al. Cancer Res 2005; 65(20): 9510-6].

In summary, the precise role of dCK in cancer cell or viral resistance to (deoxy)nucleotide or (deoxy)nucleoside analog drugs remains unclear. In connection with the current invention, the discovery that compounds of the invention may potentiate anticancer or antiviral drugs via modulation of deoxynucleotide or deoxynucleoside kinase activity, and in particular dCK, with a subsequent increased phosphorylation and bioavailability of said drugs was unexpected and could not be predicted. As a consequence, this finding defines specific patient subpopulations for whom treatment with the compound of the invention and at least one (deoxy)nucleotide or (deoxy)nucleoside analog drug can be expected to be of therapeutic benefit, i.e. patients with an under-expression, down-regulation, or decreased activity of dCK, and also patients who are intolerant to the standard dosage regimen of a given anticancer or antiviral agent. Recently, we discovered that the combination of masitinib, a small molecule inhibitor, and gemcitabine (Gemzar®, Eli Lilly and Company), a nucleoside analog, inhibits the growth of human pancreatic adenocarcinoma. Our in vitro studies established proof-of-concept that masitinib can sensitize gemcitabine-refractory pancreatic cancer cell lines (see Example 1). Masitinib as a single agent was shown to have no significant antiproliferative activity while the masitinib/gemcitabine combination showed synergy in vitro on proliferation of gemcitabine-refractory cell lines Mia Paca2 and Panc1, and to a lesser extent in vivo on Mia Paca2 cell tumor growth. Specifically, masitinib at 10 μM strongly sensitized Mia Paca2 cells to gemcitabine (400-fold reduction in IC₅₀); and moderately sensitized Panc1 cells (10-fold reduction) [Humbert M, et al. (2010) PLoS ONE 5(3): e9430. doi:10.1371/journal.pone.0009430]. These findings are supported by other in vitro data that shows masitinib can sensitize various human and canine cancer cell lines to a range of chemotherapeutic agents (see Examples 2 and 3). Masitinib sensitized different cell lines of human breast cancer, prostate cancer, ovarian cancer, colon cancer, and non-small cell lung cancer (NSCLC) to gemcitabine. Masitinib also strongly sensitized canine osteosarcoma and mammary carcinoma cells to gemcitabine [Thamm D H, et al. 2011 The Veterinary Journal, doi:10.1016/j.tvjl.2011.01.001]. These data established proof-of-concept that masitinib in combination with chemotherapeutic agents such as gemcitabine can generate synergistic growth inhibition in various human and canine cancers, possibly through chemosensitization.

Data from our in vivo studies also discovered antiproliferative activity of the masitinib/gemcitabine combination in a Nog-SCID mouse model of human pancreatic cancer (see Example 4). As expected, gemcitabine monotherapy efficiently reduced tumor growth compared to the control, while masitinib monotherapy only weakly inhibited tumor growth. The combination of masitinib plus gemcitabine also reduced tumor growth and showed an improvement in tumor inhibition as compared to gemcitabine monotherapy. These results confirm the hypothesis that masitinib can enhance the antiproliferative activity of gemcitabine in vivo.

From the masitinib-related preclinical data one could tentatively hypothesize that masitinib in combination with gemcitabine can generate synergistic growth inhibition in various cancers. In broader terms, it may be possible that small molecule inhibitors/activators (including ATP competitive inhibitors, signal transduction inhibitors/activators, protein kinase inhibitors/activators, and tyrosine kinase inhibitors/activators) in combination with anticancer or antiviral drugs, and in particular (deoxy)nucleotide and (deoxy)nucleoside analog drugs, can generate therapeutic benefits, possibly through chemosensitization. However, the mechanisms underlying this response remained to be elucidated and still required extensive pre-clinical experimentation to identify unknown targets (kinase or non kinase) of small molecule inhibition/activation that are responsible for this effect. Without such knowledge it would be impossible to predict which combinations can be expected to produce a synergistic effect.

We have discovered through experimentation using a reverse proteomic approach (see Example 5), an original property of masitinib that can account for the observed response of this drug in combination with anticancer drugs such as gemcitabine and will therefore enable the identification, development, and application of small molecule inhibitors/activators (including ATP competitive inhibitors, signal transduction inhibitors/activators, protein kinase inhibitors/activators, and tyrosine kinase inhibitors/activators) in combination therapies with anticancer or antiviral agents, especially (deoxy)nucleotide or (deoxy)nucleoside analog drugs, for the treatment of cancers (including hematological malignancies) and viral infections.

We have generated a modified version of masitinib with the following formula:

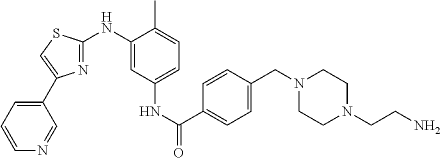 [SMILES: Cc1ccc(NC(=O)c2ccc(CN3CCN(CCN)CC3)cc2)cc1Nc1nc(-c2cccnc2)cs1]

Formula: C₂₉H₃₃N₇OS

PM: 527.68

This modified masitinib is able to be covalently coupled to NHS-beads. Beads were then incubated with cellular lysates and protein pull down were performed under proteomic conditions. After precipitation, proteins were analyzed by LC-MS and were identified by protein database comparison.

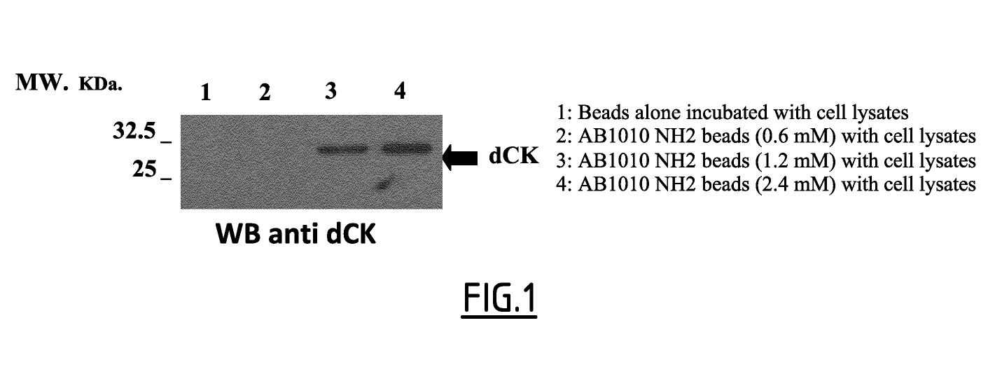

Conditions of affinity precipitations were validated on known targets (c-Kit, Lyn) and MS-spectrometry protein identifications have been obtained from various cell extracts with the same results. Protein interactions with masitinib have then been confirmed by western blot analysis using specific antibodies. Seen below (FIG. 1) is confirmation of interaction between dCK and masitinib by using western blot with anti dCK antibody after a NH2-modified-masitinib pull down.

Results have identified the deoxycytidine kinase (dCK) as being among the masitinib interacting proteins.

The direct masitinib interaction with dCK suggests an original and never described mechanism for this class of enzyme. Thus, it appears that masitinib is capable of modulating dCK activity with a consequence that it can modulate phosphorylation of (deoxy)nucleotide or (deoxy)nucleoside analog drugs. Such a property may be of great therapeutic benefit, either amplifying the effectiveness of dCK-associated chemotherapeutic agents, reducing the risk of such chemotherapeutic agents by maintaining effectiveness at lower doses, or by counteracting the effects of drug resistance. This discovery is contra-intuitive as chemotherapy resensitization could be more expected to occur due to inhibition of an enzymatic activity rather than activation of enzymatic activity.

Unexpected data showing modification of dCK enzymatic activity by masitinib is described in Example 5. Summarizing these findings, we have positively identified that the deoxynucleoside kinase dCK is one of the masitinib-interacting proteins, with masitinib effectively up-regulating its activity. Thus, it appears that masitinib is capable of modulating dCK activity with a consequence that it can induce phosphorylation of (deoxy)nucleotide or (deoxy)nucleoside analog drugs. It was also discovered that this concept is not a generally applicable to all small molecule inhibitors as the following small molecule inhibitors, and without particular limitation, did not activate dCK: dovitinib, erlotinib, fostamatinib, nilotinib, pazopanib, sorafenib, sunitinib, toceranib, and vemurafenib. However, in additional to masitinib the following small molecule inhibitors, and without particular limitation, were observed to activate dCK: imatinib, BI-2536, bosutinib, danusertib, and tozacertib

Small molecule inhibitors/activators are drugs that interfere with the function of molecules involved in the development and progression of various diseases, most commonly through the mechanisms of ATP competitive inhibition, signal transduction inhibition/activation, protein kinase inhibition/activation, or tyrosine kinase inhibition/activation. For example, a tyrosine kinase inhibitor is a drug that inhibits tyrosine kinases, thereby interfering with signaling processes within cells. Blocking such processes can stop the cell growing and dividing.

In one embodiment, the small molecule inhibitor/activator of the invention has the following formula [A]:

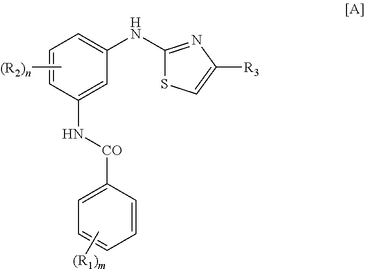

### Wherein:

R1 and R2 are selected independently from hydrogen, halogen, a linear or branched alkyl, cycloalkyl group containing from 1 to 10 carbon atoms, trifluoromethyl, alkoxy, cyano, amino, alkylamino, dialkylamino, solubilizing group.
m is 0-5 and n is 0-4.
R3 is one of the following:
(i) an aryl group such as phenyl or a substituted variant thereof bearing any combination, at any one ring position, of one or more substituents such as halogen, alkyl groups containing from 1 to 10 carbon atoms, trifluoromethyl, cyano and alkoxy;
(ii) a heteroaryl group such as 2, 3, or 4-pyridyl group, which may additionally bear any combination of one or more substituents such as halogen, alkyl groups containing from 1 to 10 carbon atoms, trifluoromethyl and alkoxy;
(iii) a five-membered ring aromatic heterocyclic group such as for example 2-thienyl, 3-thienyl, 2-thiazolyl, 4-thiazolyl, 5-thiazolyl, which may additionally bear any combination of one or more substituents such as halogen, an alkyl group containing from 1 to 10 carbon atoms, trifluoromethyl, and alkoxy, or a pharmaceutically acceptable salt or solvent thereof.

Unless otherwise specified, the below terms used herein are defined as follows:

As used herein, the term an “aryl group” means a monocyclic or polycyclic-aromatic radical comprising carbon and hydrogen atoms. Examples of suitable aryl groups include, but are not limited to, phenyl, tolyl, anthracenyl, fluorenyl, indenyl, azulenyl, and naphthyl, as well as benzo-fused carbocyclic moieties such as 5,6,7,8-tetrahydronaphthyl. An aryl group can be unsubstituted or substituted with one or more substituents.

In one embodiment, the aryl group is a monocyclic ring, wherein the ring comprises 6 carbon atoms, referred to herein as “(C6)aryl.”

As used herein, the term “alkyl group” means a saturated straight chain or branched non-cyclic hydrocarbon having from 1 to 10 carbon atoms. Representative saturated straight chain alkyls include methyl, ethyl, n-propyl, n-butyl, n-pentyl, n-hexyl, n-heptyl, n-octyl, n-nonyl and n-decyl; while saturated branched alkyls include isopropyl, sec-butyl, isobutyl, tert-butyl, isopentyl, 2-methylbutyl, 3-methylbutyl, 2-methylpentyl, 3-methylpentyl, 4-methylpentyl, 2-methylhexyl, 3-methylhexyl, 4-methylhexyl, 5-methylhexyl, 2,3-dimethylbutyl, 2,3-dimethylpentyl, 2,4-dimethylpentyl, 2,3-dimethylhexyl, 2,4-dimethylhexyl, 2,5-dimethylhexyl, 2,2-dimethylpentyl, 2,2-dimethylhexyl, 3,3-dimethylpentyl, 3,3-dimethylhexyl, 4,4-dimethylhexyl, 2-ethylpentyl, 3-ethylpentyl, 2-ethylhexyl, 3-ethylhexyl, 4-ethylhexyl, 2-methyl-2-ethylpentyl, 2-methyl-3-ethylpentyl, 2-methyl-4-ethylpentyl, 2-methyl-2-ethylhexyl, 2-methyl-3-ethylhexyl, 2-methyl-4-ethylhexyl, 2,2-diethylpentyl, 3,3-diethylhexyl, 2,2-diethylhexyl, 3,3-diethylhexyl and the like. Alkyl groups included in compounds of this invention may be optionally substituted with one or more substituents.

As used herein, the term “alkoxy” refers to an alkyl group which is attached to another moiety by an oxygen atom. Examples of alkoxy groups include methoxy, isopropoxy, ethoxy, tert-butoxy, and the like. Alkoxy groups may be optionally substituted with one or more substituents.

As used herein, the term “heteroaryl” or like terms means a monocyclic or polycyclic heteroaromatic ring comprising carbon atom ring members and one or more heteroatom ring members (such as, for example, oxygen, sulfur or nitrogen). Typically, a heteroaryl group has from 1 to about 5 heteroatom ring members and from 1 to about 14 carbon atom ring members. Representative heteroaryl groups include pyridyl, 1-oxo-pyridyl, furanyl, benzo[1,3]dioxolyl, benzo[1,4]dioxinyl, thienyl, pyrrolyl, oxazolyl, imidazolyl, thiazolyl, isoxazolyl, quinolinyl, pyrazolyl, isothiazolyl, pyridazinyl, pyrimidinyl, pyrazinyl, triazinyl, triazolyl, thiadiazolyl, isoquinolinyl, indazolyl, benzoxazolyl, benzofuryl, indolizinyl, imidazopyridyl, tetrazolyl, benzimidazolyl, benzothiazolyl, benzothiadiazolyl, benzoxadiazolyl, indolyl, tetrahydroindolyl, azaindolyl, imidazopyridyl, quinazolinyl, purinyl, pyrrolo[2,3]pyrimidinyl, pyrazolo[3,4]pyrimidinyl, imidazo[1,2-a]pyridyl, and benzo(b)thienyl. A heteroatom may be substituted with a protecting group known to those of ordinary skill in the art, for example, the hydrogen on a nitrogen may be substituted with a tert-butoxycarbonyl group. Heteroaryl groups may be optionally substituted with one or more substituents. In addition, nitrogen or sulfur heteroatom ring members may be oxidized. In one embodiment, the heteroaromatic ring is selected from 5-8 membered monocyclic heteroaryl rings. The point of attachment of a heteroaromatic or heteroaryl ring to another group may be at either a carbon atom or a heteroatom of the heteroaromatic or heteroaryl rings.

The term “heterocycle” as used herein, refers collectively to heterocycloalkyl groups and heteroaryl groups.

As used herein, the term “heterocycloalkyl” means a monocyclic or polycyclic group having at least one heteroatom selected from O, N or S, and which has 2-11 carbon atoms, which may be saturated or unsaturated, but is not aromatic. Examples of heterocycloalkyl groups including (but not limited to): piperidinyl, piperazinyl, 2-oxopiperazinyl, 2-oxopiperidinyl, 2-oxopyrrolidinyl, 4-piperidonyl, pyrrolidinyl, hydantoinyl, valerolactamyl, oxiranyl, oxetanyl, tetrahydropyranyl, tetrahydrothiopyranyl, tetrahydropyrindinyl, tetrahydropyrimidinyl, tetrahydrothiopyranyl sulfone, tetrahydrothiopyranyl sulfoxide, morpholinyl, thiomorpholinyl, thiomorpholinyl sulfoxide, thiomorpholinyl sulfone, 1,3-dioxolane, tetrahydrofuranyl, dihydrofuranyl-2-one, tetrahydrothienyl, and tetrahydro-1,1-dioxothienyl. Typically, monocyclic heterocycloalkyl groups have 3 to 7 members. Preferred 3 to 7 membered monocyclic heterocycloalkyl groups are those having 5 or 6 ring atoms. A heteroatom may be substituted with a protecting group known to those of ordinary skill in the art, for example, the hydrogen on a nitrogen may be substituted with a tert-butoxycarbonyl group. Furthermore, heterocycloalkyl groups may be optionally substituted with one or more substituents. In addition, the point of attachment of a heterocyclic ring to another group may be at either a carbon atom or a heteroatom of a heterocyclic ring. Only stable isomers of such substituted heterocyclic groups are contemplated in this definition.

As used herein the term “substituent” or “substituted” means that a hydrogen radical on a compound or group is replaced with any desired group that is substantially stable to reaction conditions in an unprotected form or when protected using a protecting group. Examples of preferred substituents are those found in the exemplary compounds and embodiments disclosed herein, as well as halogen (chloro, iodo, bromo, or fluoro); alkyl; alkenyl; alkynyl; hydroxy; alkoxy; nitro; thiol; thioether; imine; cyano; amido; phosphonato; phosphine; carboxyl; thiocarbonyl; sulfonyl; sulfonamide; ketone; aldehyde; ester; oxygen (—O); haloalkyl (e.g., trifluoromethyl); cycloalkyl, which may be monocyclic or fused or non-fused polycyclic (e.g., cyclopropyl, cyclobutyl, cyclopentyl, or cyclohexyl), or a heterocycloalkyl, which may be monocyclic or fused or non-fused polycyclic (e.g., pyrrolidinyl, piperidinyl, piperazinyl, morpholinyl, or thiazinyl), monocyclic or fused or non-fused polycyclic aryl or heteroaryl (e.g., phenyl, naphthyl, pyrrolyl, indolyl, furanyl, thiophenyl, imidazolyl, oxazolyl, isoxazolyl, thiazolyl, triazolyl, tetrazolyl, pyrazolyl, pyridyl, quinolinyl, isoquinolinyl, acridinyl, pyrazinyl, pyridazinyl, pyrimidinyl, benzimidazolyl, benzothiophenyl, or benzofuranyl); amino (primary, secondary, or tertiary); CO2CH3; CONH2; OCH2CONH2; NH2; SO2NH2; OCHF2; CF3; OCF3; and such moieties may also be optionally substituted by a fused-ring structure or bridge, for example —OCH2O—. These substituents may optionally be further substituted with a substituent selected from such groups. In certain embodiments, the term “substituent” or the adjective “substituted” refers to a substituent selected from the group consisting of an alkyl, an alkenyl, an alkynyl, an cycloalkyl, an cycloalkenyl, a heterocycloalkyl, an aryl, a heteroaryl, an aralkyl, a heteraralkyl, a haloalkyl, —C(O)NR11R12, —NR13C(O)R14, a halo, —OR13, cyano, nitro, a haloalkoxy, —C(O)R13, —NR11R12, —SR13, —C(O)OR13, —OC(O)R13, —NR13C(O)NR11R12, —OC(O)NR11R12, —NR13C(O)OR14, —S(O)rR13, —NR13S(O)rR14, —OS(O)rR14, S(O)rNR11R12, —O, —S, and —N—R13, wherein r is 1 or 2; R11 and R12, for each occurrence are, independently, H, an optionally substituted alkyl, an optionally substituted alkenyl, an optionally substituted alkynyl, an optionally substituted cycloalkyl, an optionally substituted cycloalkenyl, an optionally substituted heterocycloalkyl, an optionally substituted aryl, an optionally substituted heteroaryl, an optionally substituted aralkyl, or an optionally substituted heteraralkyl; or R1 and R12 taken together with the nitrogen to which they are attached is optionally substituted heterocycloalkyl or optionally substituted heteroaryl; and R13.0 and R14 for each occurrence are, independently, H, an optionally substituted alkyl, an optionally substituted alkenyl, an optionally substituted alkynyl, an optionally substituted cycloalkyl, an optionally substituted cycloalkenyl, an optionally substituted heterocycloalkyl, an optionally substituted aryl, an optionally substituted heteroaryl, an optionally substituted aralkyl, or an optionally substituted heteraralkyl. In certain embodiments, the term “substituent” or the adjective “substituted” refers to a solubilizing group.

The term “solubilizing group” means any group which can be substantially ionized and that enables the compound to be soluble in a desired solvent, such as, for example, water or water-containing solvent. Furthermore, the solubilizing group can be one that increases the compound or complex's lipophilicity. Typically, the solubilizing group is selected from alkyl group substituted with one or more heteroatoms such as N, O, S, each optionally substituted with alkyl group substituted independently with alkoxy, amino, alkylamino, dialkylamino, carboxyl, cyano, or substituted with cycloheteroalkyl or heteroaryl, or a phosphate, or a sulfate, or a carboxylic acid.

For example, by “solubilizing group” it is referred herein to one of the following:
an alkyl, cycloalkyl, aryl, heretoaryl group comprising either at least one nitrogen or oxygen heteroatom or which group is substituted by at least one amino group or oxo group.
an amino group which may be a saturated cyclic amino group which may be substituted by a group consisting of alkyl, alkoxycarbonyl, halogen, haloalkyl, hydroxyalkyl, amino, monoalkylamino, dialkylamino, carbamoyl, monoalkylcarbamoyl and dialkylcarbamoyl.
one of the structures a) to i) shown below, wherein the wavy line and the arrow line correspond to the point of attachment to core structure of formula A.

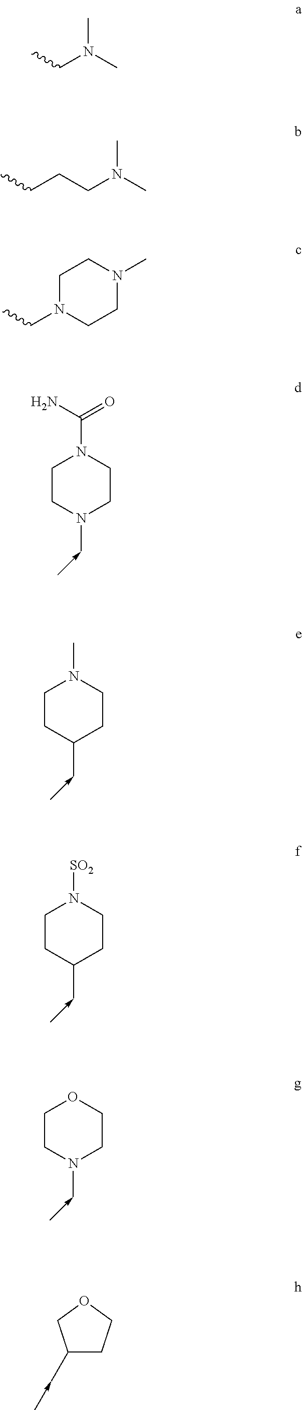

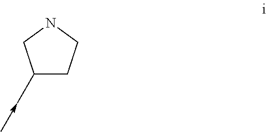

The term “cycloalkyl” means a saturated cyclic alkyl radical having from 3 to 10 carbon atoms. Representative cycloalkyls include cyclopropyl, 1-methylcyclopropyl, cyclobutyl, cyclopentyl, cyclohexyl, cycloheptyl, cyclooctyl, cyclononyl, and cyclodecyl. Cycloalkyl groups can be optionally substituted with one or more substituents.

The term “halogen” means —F, —Cl, —Br or —I.

In a particular embodiment the small molecule drug of the invention has general formula B, In a particular embodiment the invention relates to a compound of formula B, or a pharmaceutical acceptable salt thereof.

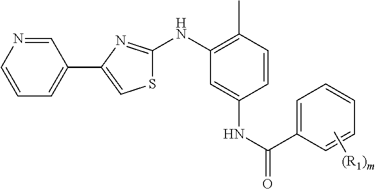

### [B]

### Wherein:

R1 is selected independently from hydrogen, halogen, a linear or branched alkyl, cycloalkyl group containing from 1 to 10 carbon atoms, trifluoromethyl, alkoxy, amino, alkylamino, dialkylamino, solubilizing group.
m is 0-5.

Masitinib is a c-Kit/FGFR3/PDGFR inhibitor with a potent anti-mast cell action

In one embodiment the small molecule inhibitor of the invention is masitinib or a pharmaceutically acceptable salt thereof, more preferably masitinib mesilate.

New potent and selective c-Kit, PDGFR and FGFR3 inhibitors are 2-(3-aminoaryl)amino-4-aryl-thiazoles described in AB Science's PCT application WO 2004/014903.

Masitinib is a small molecule drug, selectively inhibiting specific tyrosine kinases such as c-Kit, PDGFR, Lyn, Fyn and the fibroblast growth factor receptor 3 (FGFR3), without inhibiting, at therapeutic doses, kinases associated with known toxicities (i.e. those tyrosine kinases or tyrosine kinase receptors attributed to possible tyrosine kinase inhibitor cardiac toxicity, including ABL, KDR and Src) [Dubreuil et al., 2009, PLoS ONE 2009.4(9):e7258]. The chemical name for masitinib is 4-(4-methylpiperazin-1-ylmethyl)-N-[4-methyl-3-(4-pyridin-3ylthiazol-2-ylamino)phenyl]benzamide—CAS number 790299-79-5, and the structure is shown below. Masitinib was first described in U.S. Pat. No. 7,423,055 and EP1525200B1. A detailed procedure for the synthesis of masitinib mesilate is given in WO2008/098949.

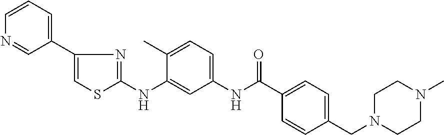 [SMILES: Cc1ccc(NC(=O)c2ccc(CN3CCN(C)CC3)cc2)cc1Nc1nc(-c2cccnc2)cs1]

Masitinib's main kinase target is c-Kit, for which it has been shown to exert a strong inhibitory effect on wild-type and juxtamembrane-mutated c-Kit receptors, resulting in cell cycle arrest and apoptosis of cell lines dependent on c-Kit signaling [Dubreuil et al., 2009, PLoS ONE, 4(9):e7258]. Stem cell factor, the ligand of the c-Kit receptor, is a critical growth factor for mast cells; thus, masitinib is an effective anti-mastocyte, exerting a direct anti-proliferative and pro-apoptotic action on mast cells through its inhibition of c-Kit signaling. In vitro, masitinib demonstrated high activity and selectivity against c-Kit, inhibiting recombinant human wild-type c-Kit with an half inhibitory concentration (IC₅₀) of 200±40 nM and blocking stem cell factor-induced proliferation and c-Kit tyrosine phosphorylation with an IC₅₀ of 150±80 nM in Ba/F3 cells expressing human or mouse wild-type c-Kit. In addition to its anti-proliferative properties, masitinib can also regulate the activation of mast cells through its targeting of Lyn and Fyn, key components of the transduction pathway leading to IgE induced degranulation [Gilfillan & Tkaczyk, 2006, Nat Rev Immunol, 6:218-230] [Gilfillan et al., 2009, Immunological Reviews, 228:149-169]. This can be observed in the inhibition of FcεRI-mediated degranulation of human cord blood mast cells [Dubreuil et al., 2009, PLoS ONE; 4(9):e7258]. Masitinib is also a potent inhibitor of PDGFR α and β receptors. Recombinant assays show that masitinib inhibits the in vitro protein kinase activity of PDGFR-α and β with IC₅₀ values of 540±60 nM and 800±120 nM. In Ba/F3 cells expressing PDGFR-α, masitinib inhibited PDGF-BB-stimulated proliferation and PDGFR-α tyrosine phosphorylation with an IC₅₀ of 300±5 nM.

Current antiviral and anticancer combination therapies consist of the treatment of patients with more than one individual therapeutic agent with the purpose to produce an additive or synergistic effect; that is to say, such combinations are more effective than the administration of the individual drugs alone. One objective of such a combination treatment approach is to increase the therapeutic efficacy. A second objective is to realize a potential decrease in dose of at least one of the individual components from the resulting combination in order to decrease unwanted or harmful side effects caused by higher doses of the individual components.

The present invention relates to a method of treating cancer (including hematological malignancies) or viral infection in a subject in need thereof, for example a human patient, by administering a first amount of at least one small molecule inhibitor/activator (including ATP competitive inhibitors, signal transduction inhibitors/activators, protein kinase inhibitors/activators, and tyrosine kinase inhibitors/activators), especially masitinib or a pharmaceutically acceptable salt or hydrate thereof, in a first treatment procedure, and a second amount of at least one anticancer or antiviral agent, especially a (deoxy)nucleotide or (deoxy)nucleoside analog drug, in a second treatment procedure, wherein the first and second amounts together comprise a therapeutically effective amount. The combined therapy of small molecule inhibitor(s)/activator(s) and (deoxy)nucleotide or (deoxy)nucleoside analog drug(s) produce a therapeutically beneficial anticancer or antiviral effect, for example, a synergistic effect.

In relation to the present invention, the term “treating” (and its various grammatical forms) refers to preventing, curing, reversing, attenuating, alleviating, minimizing, suppressing or halting the deleterious effects of a disease state, disease progression, disease causative agent (e.g., bacteria or viruses) or other abnormal condition. For example, treatment may involve alleviating a symptom (i.e., not necessary all symptoms) of a disease or attenuating the progression of a disease.

As used herein, the term “therapeutically effective amount” is intended to qualify the combined amount of the first and second treatments in the combination therapy. The combined amount will achieve the desired biological response. In one embodiment of the present invention, the desired biological response is partial or total inhibition, delay or prevention of the progression of cancer including cancer metastasis; inhibition, delay or prevention of the recurrence of cancer including cancer metastasis. In another embodiment of the present invention, the desired biological response is delay or prevention of the progression of viral infection including a partial or total block of viral replication; reduced viral load or a viral load maintained at undetectable levels; increased immune function and improved health status (including for example but not restricted to: prevention or decreased incidence of opportunistic infections and malignancies, increase in CD4 counts, stamina, and weight gain).

In relation to the present invention, the term “synergistic” (and its various grammatical forms) refers to the capacity of two or more drugs acting together so that the total effect of these drugs is greater than the sum of the effects if taken independently. The presence and effects of one drug enhances the effects of the second.

As used herein, the terms “combination treatment”, “combination therapy”, “combined treatment” or “combinatorial treatment”, used interchangeably, refer to a treatment of an individual with at least two different therapeutic agents. According to the invention, the individual is treated with a first therapeutic agent, a small molecule inhibitor/activator as described herein, especially masitinib or a pharmaceutically acceptable salt or hydrate thereof. The second therapeutic agent is an anticancer or antiviral agent, especially a (deoxy)nucleotide or (deoxy)nucleoside analog drug. A combinatorial treatment may include a third or even further therapeutic agents. The compound(s) of the invention and one or more anticancer or antiviral agent may be administered separately, simultaneously or sequentially in time.

The invention further relates to pharmaceutical combinations useful for the treatment of cancer (including hematological malignancies) or viral infections. The pharmaceutical combination comprises a first amount of at least one small molecule inhibitor/activator, especially masitinib or a pharmaceutically acceptable salt or hydrate thereof, and a second amount of at least one anticancer or antiviral agent, especially a (deoxy)nucleotide or (deoxy)nucleoside analog drug. The first and second amount together comprises a therapeutically effective amount. The invention further relates to the use of a first amount of at least one small molecule inhibitor/activator, especially masitinib or a pharmaceutically acceptable salt or hydrate thereof, and a second amount of at least one anticancer or antiviral agent, especially a (deoxy)nucleotide or (deoxy)nucleoside analog drug, for the manufacture of a medicament for treating cancer (including hematological malignancies) or viral infection. In particular embodiments of this invention, the combination of at least one small molecule inhibitor/activator, especially masitinib or a pharmaceutically acceptable salt or hydrate thereof, and a second amount of at least one anticancer or antiviral agent, especially a (deoxy)nucleotide or (deoxy)nucleoside analog drug, is considered therapeutically synergistic when the combination treatment regimen produces a better anticancer or antiviral result (e.g., cell growth arrest, apoptosis, induction of differentiation, cell death, inhibited viral reproduction, reduced viral load, improved immune function) than the additive effects of each constituent when it is administered alone at the corresponding dosages.

The invention also relates to the use of at least one small molecule inhibitor/activator in combination with at least one anticancer or antiviral drug for the preparation of a medicament, or a pharmaceutical composition, for the treatment of a cancer (including hematological malignancies) or viral infection, as defined in the present description and examples.

The invention also relates to a small molecule inhibitor/activator in combination with at least one anticancer or antiviral drug for use in a method for the treatment of a cancer (including hematological malignancies) or viral infection as defined in the present description and examples.

The invention also relates to a pharmaceutical composition or kit comprising at least one small molecule inhibitor/activator in combination with at least one anticancer or antiviral drug for use in a method for the treatment of a cancer (including hematological malignancies) or viral infection as defined in the present description and examples.

By “kit” it is meant physically at least two separate pharmaceutical compositions, wherein one composition comprises at least one anticancer or antiviral drug and a second composition comprising at least one small molecule inhibitor/activator.

A wide variety of cancers (including hematological malignancies) may be treated by the methods of the invention including, but not limited to: acute lymphocytic leukemia (ALL), acute myelogenous leukemia (AML), adrenocortical carcinoma, anal cancer, B cell lymphoma, basal cell carcinoma, bile duct cancer, bladder cancer, bone cancer, brainstem glioma, brain tumor, breast cancer, cervical cancer, chronic lymphocytic leukemia (CLL), chronic myelogenous leukemia (CML), colorectal cancer (CRC), endometrial cancer, esophageal cancer, eye cancer, gallbladder cancer, gastric (stomach) cancer, gastrointestinal stromal tumor (GIST), glioblastoma multiforme (GBM), hairy cell leukemia, head and neck cancer, heart cancer, hepatocellular (liver) carcinoma (HCC), Hodgkin's lymphoma and non-Hodgkin's lymphomas, Kaposi sarcoma, laryngeal cancer, mastocytosis, melanoma, myelofibrosis, myelodysplastic disease, myeloproliferative disease, myeloproliferative neoplasms, hematological neoplasms, myelodysplastic syndrome (MDS), multiple myeloma, non-small-cell lung carcinoma (NSCLC), lung cancer (small cell), melanoma, nasopharyngeal carcinoma, neuroendocrine tumors, neuroblastoma, oral cancer, oropharyngeal cancer, ovarian cancer, pancreatic cancer, paranasal sinus and nasal cavity cancer, parathyroid cancer, penile cancer, pharyngeal cancer, pituitary adenoma, prostate cancer, rectal cancer, renal cell (kidney) carcinoma (RCC), salivary gland cancer, skin cancer (nonmelanoma), small intestine cancer, small lymphocytic lymphoma (SSL), soft tissue sarcoma, squamous-cell carcinoma, T cell lymphoma, testicular cancer, throat cancer, thyroid cancer, triple negative breast cancer, urethral cancer, and uterine cancer.

Other cancers embraced by the methods of the present invention are: colon cancer, lung cancer, brain cancer, testicular cancer, skin cancer, small intestine cancer, large intestine cancer, throat cancer, oral cancer, bone cancer, thyroid cancer, hematological cancers, lymphoma and leukemia. Cancers that may be treated by the methods of the invention include, but are not limited to: Cardiac: sarcoma (angiosarcoma, fibrosarcoma, rhabdomyosarcoma, liposarcoma), myxoma, rhabdomyoma, fibroma, lipoma and teratoma; Lung: bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, mesothelioma; Gastrointestinal: esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Karposi's sarcoma, leiomyoma, hemangioma, lipoma, neurofibroma, fibroma), large bowel (adenocarcinoma, tubular adenoma, villous adenoma, hamartoma, leiomyoma), colon, colorectal, rectal; Genitourinary tract: kidney (adenocarcinoma, Wilm's tumor [nephroblastoma], lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma); Liver: hepatoma (hepatocellular carcinoma), cholangiocarcinoma, hepatoblastoma, angiosarcoma, hepatocellular adenoma, hemangioma; Bone: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors; Nervous system: skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma [pinealoma], glioblastoma multiform, oligodendroglioma, schwannoma, retinoblastoma, congenital tumors), spinal cord neurofibroma, meningioma, glioma, sarcoma); Gynecological: uterus (endometrial carcinoma), cervix (cervical carcinoma, pre-tumor cervical dysplasia), ovaries (ovarian carcinoma [serous cystadenocarcinoma, mucinous cystadenocarcinoma, unclassified carcinoma], granulosa-thecal cell tumors, Sertoli-Leydig cell tumors, dysgerrninoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma), breast; Hematologic: blood (myeloid leukemia [acute and chronic], acute lymphoblastic leukemia, chronic lymphocytic leukemia, myeloproliferative diseases, multiple myeloma, myelodysplastic syndrome), Hodgkin's disease, non-Hodgkin's lymphoma [malignant lymphoma]; Skin: malignant melanoma, basal cell carcinoma, squamous cell carcinoma, Karposi's sarcoma, moles dysplastic nevi, lipoma, angioma, dermatofibroma, keloids, psoriasis; and Adrenal glands: neuroblastoma.

The methods of the present invention are useful in the treatment in a wide variety of viral infections, including but not limited to: human immunodeficiency virus (HIV) infections, acquired immune deficiency syndrome (AIDS), hepacivirus infections (including hepatitis B, hepatitis C), herpes simplex virus (including HSV-1, HSV-2), varicella-zoster virus (VZV), human cytomegalovirus (HCMV), human papilloma virus (HPV), Epstein-Barr virus (EBV), Kaposi's sarcoma-associated herpes virus (KSHV), DNA virus infections, orthomyxovirus infections (i.e., influenza), viral hemorrhagic fevers (VHF), flaviviridae viruses (including West Nile virus, dengue virus, tick-borne encephalitis virus, yellow fever virus), or SARS coronavirus.

In particular, said at least one small molecule inhibitor/activator is administered in combination with at least one of said (deoxy)nucleotide or (deoxy)nucleoside analog drugs for the treatment patients suffering from cancer (including hematological malignancies) or viral infection, selected from the above indications.

In the present invention as defined above, the small molecule inhibitor/activator, dosed ideally in accordance to the manufacture's recommendations, is for example, and without particular limitation, either: afatinib, alitretinoin, axitinib, bafetinib, bexarotene, BI-2536, bosutinib, brivanib, canertinib, cediranib, CP724714, crizotinib, dasatinib, danusertib, dovitinib, E7080, erlotinib, everolimus, fostamatinib, gefitinib, imatinib, lapatinib, lestaurtinib, linsitinib, masitinib, motesanib, neratinib, nilotinib, NVP TAE-684, OSI-027, OSI-420, OSI-930, pazopanib, pelitinib, PF573228, regorafenib, romidepsin, ruxolitinib, saracatinib, sorafenib, sunitinib, TAE226, TAE684, tandutinib, telatinib, tautinib, temsirolimus, toceranib, tofacitinib, tozasertib, tretinoin, vandetanib, vatalanib, vemurafenib, vorinostat and WZ 4002.

A representative list of small molecule inhibitors/activators is presented in Tables 1 and 2. Many other small molecule inhibitors/activators are in development.

In one embodiment of the above-depicted treatment, the small molecule inhibitor/activator is chosen from masitinib, imatinib, sunitinib, axitinib, bosutinib, tozasertib, saracatinib, BI-2536, or NVP TAE-684.

In the present invention as defined above, the anticancer or antiviral agent is for example, and without particular limitation, either: abacavir, acyclovir, adefovir, amdoxovir, apricitabine, azacitidine, Atripla®, capecitabine, cladribine, movectro, clevudine, clofarabine, evoltra, Combivir®, cytarabine, decitabine, didanosine, elvucitabine, emtricitabine, entecavir, Epziconn®, festinavir, fludarabine, fluorouracil, gemcitabine, idoxuridine, KP-1461, lamivudine, nelarabine, racivir, ribavirin, sapacitabine, stavudine, taribavirin, telbivudine, tenofovir, tezacitabine, trifluridine, Trizivir®, troxacitabine, Truvada®, vidarabine, zalcitabine, or zidovudine.

A representative list of anticancer and antiviral agents, including (deoxy)nucleotide and (deoxy)nucleoside analog drugs, is presented in Tables 3 and 4. Many other anticancer and antiviral agents are in development.

TABLE 1
Representative examples of small molecule inhibitors/activators and their uses.

| | | | | Regulatory |
|---|---|---|---|---|
| NAME (INN) | BRAND | COMPANY | Indications | status |
| Alitretinoin | Panretin ® | Ligand | AIDS-related Kaposi sarcoma | FDA approved |
| | | Pharmaceuticals | | |
| Afatinib | Tomtovok ® | Boehringer | Solid tumors (inc. NSCLC, breast, | Phase 2/3 |
| | | Ingelheim | prostate) | |
| Axitinib | | Pfizer | Solid tumors (inc. breast, RCC) | Phase 2/3 |
| Bexarotene | Targretin ® | Eisai | CTCL | FDA approved |
| BI-2536 | | Boehringer | Solid tumors | Phase 2/3 |
| | | Ingelheim | | |
| Bosutinib | | Wyeth | Solid/hematological cancers (inc. | Phase 2/3 |
| | | | breast, CML), | |
| Brivanib | | BMS | Solid tumors (inc. HCC) | Phase 2/3 |
| Canertinib | | Pfizer | Solid/hematological cancers | Phase 2/3 |
| Cediranib | Recentin ® | AstraZeneca | Solid tumors | Phase 2/3 |
| CP 724714 | | Pfizer | Solid tumors | Phase 1 |
| Crizotinib | Xalkori ® | Pfizer | Solid tumors (inc. NSCLC) | FDA approved |
| Dasatinib | Sprycel ® | BMS | CML (blast phase, chronic phase), | FDA approved |
| | | | Acute lymphoblastic leukemia | |
| E7080 | | Eisai | Solid tumors | Phase 2/3 |
| Erlotinib | Tarceva ® | OSI | Solid tumors (inc. NSCLC, pancreatic) | FDA approved |
| Everolimus | Afinitor ® | Novartis | NSCLC | FDA approved |
| Fostamatinib | | AstraZeneca | Rheumatoid arthritis | Phase 2/3 |
| Gefitinib | Iressa ® | AstraZeneca | Solid tumors (inc. NSCLC) | FDA approved |
| Imatinib | Gleevec ® | Novartis | Hematological malignancy, solid | FDA approved |
| | | | tumors (inc. CML, GIST, systemic | |
| | | | mastocytosis) | |
| Lapatinib | Tykerb ® | GSK | Solid tumors (inc. breast), | FDA approved |
| Lestaurtinib | | Cephalon | Hematological malignancy (inc. AML) | Phase 2/3 |
| Linsitinib (OSI | | OSI | Solid/hematological cancers | Phase 2/3 |
| 906) | | | | |
| Masitinib | Masivet ® | AB Science | Canine mast cell tumor | FDA approved |
| | Kinavet ® | | | (vet) |
| | | | | Phase 2/3 |
| Neratinib | | Wyeth | Solid tumors (inc. breast) | Phase 2/3 |
| Nilotinib | Tasigna ® | Novartis | Hematological malignancy (inc. CML) | FDA approved |
| NVP-TAE684 | | Novartis | Solid tumors (inc. NSCLC) | Phase 1 |
| OSI-027 | | OSI | Solid tumors | Phase 1 |
| OSI 420 | | OSI | Solid tumors | Phase 1 |
| OSI 930 | | OSI | Solid tumors | Phase 1 |
| Pazopanib | Votrient ® | GSK | Solid tumors (inc. RCC, ovarian, soft | FDA approved |
| | | | tissue sarcoma.) | |
| Pelitinib | | Wyeth | Solid tumors | Phase 2/3 |
| PF573228 | | Pfizer | Solid tumors | Phase 1 |
| Regorafenib | | Bayer | Solid tumors (inc. GIST, colorectal) | Phase 2/3 |
| Romidepsin | Istodax ® | Celgene | CTCL | FDA approved |
| Ruxolitinib | | Novartis | Hematological malignancy (inc. | Phase 2/3 |
| | | | myelofibrosis) | |
| Saracatinib | | BioVision | Hematological malignancy (inc. | Phase 2/3 |
| | | | myelofibrosis). Solid cancers (inc. | |
| | | | ovarian) | |
| Sorafenib | Nexavar ® | Bayer | Solid tumors (inc. RCC, HCC) | FDA approved |
| Sunitinib | Sutent ® | Pfizer | Solid tumors (inc. GIST, RCC, | FDA approved |
| | | | pancreatic neuroendocrine tumors) | |
| Tandutinib | | Millennium | Solid/hematological cancers (inc. | Phase 2/3 |
| | | | AML, RCC) | |
| Telatinib | | ACT Biotech | Solid tumors (inc. gastric) | Phase 2/3 |
| Temsirolimus | Torisel ® | Wyeth | Advanced RCC | FDA approved |
| Toceranib | Palladia ® | Pfizer | Canine mast cell tumor | FDA approved |
| | | | | (vet) |
| Tofacitinib | | Pfizer | Immunological diseases (inc. | Phase 2/3 |
| | | | rheumatoid arthritis, psoriasis | |
| Tretinoin | Vesanoid ® | Roche | Acute promyelocytic leukemia | FDA approved |
| Vandetanib | Zactima ® | AstraZeneca | Solid tumors (inc. MTC) | FDA approved |
| Vatalanib | | Novartis | Solid tumors | Phase 2/3 |
| Vorinostat | Zolinza ® | Patheon | CTCL | FDA approved |
| WZ 4002 | | | Solid tumors (inc. lung) | Phase 1 |

ABL=Abelson proto-oncogene; ALK=anaplastic lymphoma kinase; AML=acute myelogenous leukemia; CML=chronic myelogenous leukemia; CRC=colorectal cancer; CTCL=cutaneous T-cell lymphoma; EGFR=epidermal growth factor receptor; FGFR=fibroblast growth factor receptor; GIST=gastrointestinal stromal tumor; HCC=hepatocellular carcinoma; HER2=Human EGFR type 2; HGFR=hepatocyte growth factor receptor; IGF-1R=insulin-like growth factor-1 receptor; INN=International Nonproprietary Name; IR=insulin receptor; MTC=Medullary thyroid cancer; NSCLS=Non-small-cell lung carcinoma; PDGFR=platelet-derived growth factor receptor; Plk1=Polo-Like Kinase 1; RCC=renal cell carcinoma; Trk=neurotrophic tyrosine kinase receptor; VEGFR=vascular endothelial growth factor receptor.

TABLE 2
Representative examples of small molecule inhibitors/activators and their chemical formula.

| NAME (INN) | Formula | Systematic (IUPAC) name |
|---|---|---|
| Alitretinoin | C20H28O2 | (2E,4E,6Z,8E)-3,7-dimethyl-9-(2,6,6-trimethyl- |
| | | 1-cyclohexenyl)nona-2,4,6,8-tetraenoic acid |
| Afatinib | C24H25ClFN5O3 | N-[4-[(3-Chloro-4-fluorophenyl)amino]-7-[[(3S)-tetrahydro-3- |
| | | furanyl]oxy]-6-quinazolinyl]-4(dimethylamino)-2-butenamide |
| Axitinib | C22H18N4OS | N-Methyl-2-[[3-[(E)-2-pyridin-2-ylethenyl]-1H-indazol-6- |
| | | yl]sulfanyl]benzamide |
| Bexarotene | C24H28O2 | 4-[1-(3,5,5,8,8-pentamethyltetralin-2-yl)ethenyl] |
| | | benzoic acid |
| BI-2536 | C28H39N7O3 | 4-((R)-8-cyclopentyl-7-ethyl-5,6,7,8-tetrahydro-5-methyl-6-oxopteridin- |
| | | 2-ylamino)-3-methoxy-N-(1-methylpiperidin-4-yl)benzamide |
| Bosutinib | C26H29Cl2N5O3 | 4-[(2,4-dichloro-5-methoxyphenyl)amino]-6-methoxy-7-[3-(4- |
| | | methylpiperazin-1-yl)propoxy]quinoline-3-carbonitrile |
| Brivanib | C19H19FN4O3 | 1-[[4-[(4-Fluoro-2-methyl-1H-indol-5-yl)oxy]-5-methylpyrrolo[2,1- |
| | | f][1,2,4]triazin-6-yl]oxy]-2-propanol |
| Canertinib | C24H25ClFN5O3 | N-[4-(3-Chloro-4-fluorophenylamino)-7-[3-(4- |
| | | morpholinyl)propoxy]quinazolin-6-yl]-2-propenamide dihydrochloride |
| Cediranib | C25H27FN4O3 | 4-[(4-fluoro-2-methyl-1H-indol-5-yl)oxy]-6-methoxy-7-[3-(pyrrolidin-1- |
| | | yl)propoxy]quinazoline |
| CP 724714 | C27H27N5O3 | 2-Methoxy-N-[3-[4-[[3-methyl-4-[(6-methyl-3- |
| | | pyridinyl)oxy]phenyl]amino]-6-quinazolinyl]-2-propen-1-yl]acetamide |
| Crizotinib | C21H22Cl2FN5O | 3-[(1R)-1-(2,6-dichloro-3-fluorophenyl)ethoxy]-5-(1-piperidin-4- |
| | | ylpyrazol-4-yl)pyridin-2-amine |
| Dasatinib | C22H26ClN7O2S | N-(2-chloro-6-methylphenyl)-2-[[6-[4-(2-hydroxyethyl)-1-piperazinyl]- |
| | | 2-methyl-4-pyrimidinyl]amino]-5-thiazole carboxamide monohydrate |
| E7080 | C21H19ClN4O4 | 4-[3-chloro-4-(cyclopropylcarbamoylamino)phenoxy]-7-methoxy- |
| | | quinoline-6-carboxamide |
| Erlotinib | C22H23N3O4 | N-(3-ethynylphenyl)-6,7-bis(2-methoxyethoxy) quinazolin-4-amine |
| Everolimus | C53H83NO14 | dihydroxy-12-[(2R)-1-[(1S,3R,4R)-4-(2-hydroxyethoxy)-3- |
| | | methoxycyclohexyl]propan-2-yl]-19,30-dimethoxy-15,17,21,23,29,35- |
| | | hexamethyl-11,36-dioxa-4-azatricyclo[30.3.1.04,9]hexatriaconta- |
| | | 16,24,26,28-tetraene-2,3,10,14,20-pentone |
| Fostamatinib | C23H26FN6O9P | [6-({5-fluoro-2-[(3,4,5-trimethoxyphenyl)amino]pyrimidin-4-yl}amino)- |
| | | 2,2-dimethyl-3-oxo-2,3-dihydro-4H-pyrido[3,2-b][1,4]oxazin-4-yl]methyl |
| | | dihydrogen phosphate |
| Gefitinib | C22H24ClFN4O3 | N-(3-chloro-4-fluoro-phenyl)-7-methoxy-6-(3-morpholin-4- |
| | | ylpropoxy)quinazolin-4-amine |
| Imatinib | C29H31N7O | 4-[(4-methylpiperazin-1-yl)methyl]-N-[4-methyl-3-[(4-pyridin-3- |
| | | ylpyrimidin-2-yl)amino]phenyl]benzamide |
| Lapatinib | C29H26ClFN4O4S | N-[3-chloro-4-[(3-fluorophenyl)methoxy] phenyl]-6-[5-[(2- |
| | | methylsulfonylethylamino) methyl]-2-furyl] quinazolin-4-amine |
| Lestaurtinib | C26H21N3O4 | |
| Linsitinib | C26H23N5O | Cyclobutanol, 3-[8-amino-1-(2-phenyl-7-quinolinyl)imidazo[1,5- |
| (OSI 906) | | a]pyrazin-3-yl]-1-methyl, cis- |
| Masitinib | C28H30N6OS | 4-(4-methylpiperazin-1-ylmethyl)-N-[4-methyl-3-(4-pyridin-3ylthiazol-2- |
| | | ylamino) phenyl]benzamide |
| Neratinib | C30H29ClN6O3 | (2E)-N-[4-[[3-chloro-4-[(pyridin-2-yl)methoxy] phenyl]amino]-3-cyano- |
| | | 7-ethoxyquinolin-6-yl]-4-(dimethylamino)but-2-enamide |
| Nilotinib | C28H22F3N7O | 4-methyl-N-[3-(4-methyl-1H-imidazol-1-yl)-5-(trifluoromethyl)phenyl]- |
| | | 3-[(4-pyridin-3-ylpyrimidin-2-yl) amino]benzamide |
| NVP-TAE684 | C₃₀H₄₀ClN₇O₃S | 5-Chloro-N4-(2-(isopropylsulfonyl)phenyl-N2-(2-methoxy-4-(4- |
| | | methylpiperazin-1-yl)-piperidin-1-yl)phenyl)pyrimidine-2,4-diamine |
| OSI-027 | C21H23ClN6O3 | 4-(4-amino-5-(7-methoxy-1H-indol-2-yl)imidazo[5,1-f][1,2,4]triazin-7- |
| | | yl)cyclohexanecarboxylic acid hydrochloride. |
| OSI 420 | C21H21N3O4 | 2-[[4-[(3-Ethynylphenyl)amino]-7-(2-methoxyethoxy)-6- |
| | | quinazolinyl]oxy]ethanol |
| OSI 930 | C22H16F3N3O2S | 3-[(Quinolin-4-ylmethyl)-amino]-thiophene-2-carboxylic acid (4- |
| | | trifluoromethoxy-phenyl)-amide |
| Pazopanib | C21H23N7O2S | 5-[[4-[(2,3-Dimethyl-2H-indazol-6-yl)methylamino]-2- |
| | | pyrimidinyl]amino]-2-methylbenzolsulfonamide |
| Pelitinib | C24H23ClFN5O2 | (2E)-N-{4-[(3-chloro-4-fluorophenyl)amino]-3-cyano-7-ethoxyquinolin- |
| | | 6-yl}-4-(dimethylamino)but-2-enamide |
| PF573228 | C22H20F3N5O3S | 3,4-Dihydro-6-[[4-[[[3-(methylsulfonyl)phenyl]methyl]amino]- |
| | | 5-(trifluoromethyl)-2-pyrimidinyl]amino]- |
| | | 2(1H)-quinolinone |
| Regorafenib | C21H15ClF4N4O3 | 4-[4-({[4-Chloro-3-(trifluoromethyl)phenyl]carbamoyl}amino)-3- |
| | | fluorophenoxy]-N-methylpyridine-2-carboxamide |
| Romidepsin | C24H36N4O6S2 | (1S,4S,7Z,10S,16E,21R)-7-ethylidene-4,21-diisopropyl-2-oxa-12,13- |
| | | dithia-5,8,20,23-tetrazabicyclo[8.7.6]tricos-16-ene-3,6,9,19,22-pentone |
| Ruxolitinib | C17H18N6 | (3R)-3-cyclopentyl-3-[4-(7H-pyrrolo[2,3-d]pyrimidin-4-yl)pyrazol-1- |
| | | yl]propanenitrile |
| Saracatinib | C27H32ClN5O5 | N-(5-chloro-1,3-benzodioxol-4-yl)-7-[2-(4-methylpiperazin-1-yl)ethoxy]- |
| | | 5-(tetrahydro-2H-pyran-4-yloxy)quinazolin-4-amine |
| Sorafenib | C21H16ClF3N4O3 | 4-[4-[[4-chloro-3-(trifluoromethyl)phenyl] carbamoylamino]phenoxy]-N- |
| | | methyl-pyridine-2-carboxamide |
| Sunitinib | C22H27FN4O2 | N-(2-diethylaminoethyl)-5-[(Z)-(5-fluoro-2-oxo-1H-indol-3- |
| | | ylidene)methyl]-2,4-dimethyl-1H-pyrrole-3-carboxamide |
| Tandutinib | C31H42N6O4 | 4-[6-Methoxy-7-(3-piperidin-1-ylpropoxy) quinazolin-4-yl]-N-(4-propan- |
| | | 2-yloxyphenyl) piperazine-1-carboxamide |
| Telatinib | C31H43N3O8 | 17-Demethoxy-17-allylaminogeldanamycin; Tanespimycin; 17- |
| | | Allylaminogeldanamycin |
| Temsirolimus | C56H87NO16 | |
| Toceranib | C22H25FN4O2 | (Z)-5-[(5-Fluoro-2-oxo-1,2-dihydro-3H-indol-3-ylidene)methyl]-2,4- |
| | | dimethyl-N-(2-pyrrolidin-1-ylethyl)-1H-pyrrole-3-carboxamide |
| Tofacitinib | C16H20N6O | 3-[(3R,4R)-4-methyl-3-[methyl(7H-pyrrolo[2,3-d]pyrimidin-4- |
| | | yl)amino]piperidin-1-yl]-3-oxopropanenitrile |
| Tretinoin | C20H28O2 | retinoic acid |
| Vandetanib | C22H24BrFN4O2 | N-(4-bromo-2-fluorophenyl)-6-methoxy-7-[(1-methylpiperidin-4- |
| | | yl)methoxy]quinazolin-4-amine |
| Vatalanib | C20H15ClN4 | N-(4-chlorophenyl)-4-(pyridin-4-ylmethyl)phthalazin-1-amine |
| Vorinostat | C14H20N2O3 | N-hydroxy-N′-phenyl-octanediamide |
| WZ 4002 | C25H27ClN6O3 | Chemical Name: N-(3-((5-chloro-2-((2-methoxy-4-(4-methylpiperazin-1- |
| | | yl)phenyl)amino)pyrimidin-4-yl)oxy)phenyl)acrylamide |

TABLE 3
Representative examples of anticancer and antiviral agents and their uses.

| | | | | | Regulatory |
|---|---|---|---|---|---|
| NAME (INN) | BRAND | COMPANY | Typical Dosage* | Treatment | Status |
| Abacavir | Ziagen ® | GSK | 300 mg twice daily or | Antiretroviral (HIV) | FDA |
| | | | 600 mg once daily | | approved |
| Acyclovir | Zovirax ® | | 400-800 mg tablet | Antiviral (inc. herpes viruses, | FDA |
| | | | | varicella-zoster, Epstein-Barr | approved |
| | | | | virus) | |
| Adefovir | Hepsera ® | Gilead | 10 mg once daily | Antiretroviral (inc. hepatitis | FDA |
| | | Sciences | | B, herpes) | approved |
| Amdoxovir | | RFS Pharma | | Antiretroviral (HIV) | Phase 2/3 |
| Apricitabine | | Avexa | | Antiretroviral (HIV) | Phase 2/3 |
| Azacitidine | Vidaza ® | Celgene | 75 mg/m² daily i.v. | Anticancer (inc. MDS) | FDA |
| | | | | | approved |
| | Atripla ® | Gilead | efavirenz 600 mg, | Antiretroviral (HIV) | FDA |
| | | | tenofovir 300 mg, | | approved |
| | | | emtricitabine 200 mg | | |
| Capecitabine | Xeloda ® | Roche | 1250 mg/m² b.i.d. | Anticancer (inc. breast, | FDA |
| | | | | colorectal) | approved |
| Cladribine | Litak ® | EMD Serono | 0.14 mg/kg BW i.v.; | Anticancer (inc. hairy cell | FDA |
| (2CDA) | Movectro | | | leukemia) | approved |
| Clevudine | Levovir/ | Pharmasset | | Antiretroviral (inc. hepatitis | Phase 2/3 |
| | Revovir ® | | | B) | |
| Clofarabine | Clolar ® | Genzyme | 52 mg/m² daily | Anticancer (inc. ALL, AML) | FDA |
| | (US) | Corp. | | | approved |
| | Evoltra | | | | |
| | Combivir ® | GSK | zidovudine 300 mg | Antiretroviral (HIV) | FDA |
| | | | lamivudine 150 mg | | approved |
| Cytarabine (Ara-C) | Tarabine | Pfizer | 200 mg/m² i.v. or | Anticancer (inc. ALL, AML, | FDA |
| | PFS ® | | 3000 mg/m² i.v. high | non-Hodgkin lymphoma) | approved |
| | | | dose | | |
| Decitabine | Dacogen ® | MGI Pharma | | Anticancer (inc. MDS) | FDA |
| | | | | | approved |
| Didanosine | Videx ® | BMS | 250 mg-400 mg once | Antiretroviral (HIV) | FDA |
| | | | daily p.o. | | approved |
| Elvucitabine | | Achillion | 10 mg once daily | Antiretroviral (HIV) | Phase 2/3 |
| Emtricitabine | Emtriva ® | Gilead | 200 mg once daily | Antiretroviral (HIV, hepatitis | FDA |
| | | | p.o. | B) | approved |
| Entecavir | Baraclude ® | BMS | | Antiretroviral (hepatitis B) | FDA |
| | | | | | approved |
| | Epzicom ® | GSK | 600 mg abacavir 300 mg | Antiretroviral (HIV) | FDA |
| | | | lamivudine | | approved |
| Festinavir | | BMS | | Antiretroviral (HIV) | Phase 2/3 |
| Fludarabine | Fludara ® | Genzyme | 25 mg/m² i.v | Anticancer (inc. chronic | FDA |
| | | | | lymphocytic leukemia non- | approved |
| | | | | Hodgkins lymphomas, AML) | |
| Fluorouracil | Adrucil ® | Teva | 500-2600 mg/m² i.v. | Anticancer (inc. colorectal, | FDA |
| | | | | pancreatic, breast, basal cell | approved |
| | | | | carcinoma) | |
| Gemcitabine | Gemzar ® | Eli Lilly | 1000-1250 mg/m² i.v. | Anticancer (inc. NSCLC, | FDA |
| | | | | pancreatic, bladder, breast, | approved |
| | | | | lung, esophageal) | |
| Idoxuridine | Dendrid ® | | | Antiviral (herpes) | FDA |
| | | | | | approved |
| KP-1461 | | Koronis | | Antiretroviral (HIV) | Phase 2/3 |
| Lamivudine | Zeffix, | GSK | 150 mg twice daily or | Antiretroviral (HIV, hepatitis | FDA |
| | Heptovir, | | 300 mg once daily | B) | approved |
| | Epivir ® | | | | |
| Nelarabine | Arranon ®, | GSK | 650-1500 mg/m² i.v. | Anticancer (inc. T-cell ALL | FDA |
| | Atriance | | | and T-cell lymphoblastic | approved |
| | | | | lymphoma) | |
| Racivir | | Pharmasset | 600 mg daily | Antiretroviral (HIV) | Phase 2/3 |
| Ribavirin | Virazole ® | Valeant | 800 mg to 1200 mg | Antiretroviral (hepatitis C) | FDA |
| | | Pharma | b.i.d. | | approved |
| Sapacitabine | | Cyclacel | | Anticancer (inc. AML, CLL, | Phase 2/3 |
| | | Pharma | | SLL, NSCLC,) | |
| Stavudine | Zerit ® | BMS | 30-40 mg twice daily | Antiretroviral (HIV) | FDA |
| | | | | | approved |
| Taribavirin | | Valeant | | Antiretroviral (inc. hepatitis | Phase 2/3 |
| | | Pharma | | C, hepatitis B, yellow fever) | |
| Telbivudine | Tyzeka ®, | Novartis | | Antiretroviral (hepatitis B) | Phase 2/3 |
| | Sebivo ® | | | | |
| Tenofovir | Viread ® | Gilead | 300 mg once daily | Antiretroviral (HIV) | FDA |
| | | | | | approved |
| Tezacitabine | | Chiron | | Anticancer (solid cancer inc. | Phase 2/3 |
| | | | | esophageal, stomach, | |
| | | | | Adenocarcinoma, colorectal) | |
| Trifluridine | Viroptic ® | GSK | | Antiviral (inc. herpes simplex; | Phase 2/3 |
| | | | | HIV; mycobacterium avium- | |
| | | | | intracellulare) | |
| | Trizivir ® | GSK | 300 mg abacavir | Antiretroviral (HIV) | FDA |
| | | | 150 mg Lamivudine | | approved |
| | | | 300 mg zidovudine | | |
| Troxacitabine | Troxatyl ® | SGX | | Anticancer (inc. AML, CML) | Phase 2/3 |
| | Truvada ® | Gilead | 300 mg Tenofovir | Antiretroviral (HIV) | FDA |
| | | | 200 mg Emtricitabine | | approved |
| Vidarabine | Vira-A ® | | 0.75 mg three times | Antiviral (inc. herpes simplex, | FDA |
| | | | daily | varicella zoster, vaccinia) | approved |
| Zalcitabine | Hivid ® | Roche | | Antiretroviral (HIV, AIDS) | FDA |
| | | | | | approved |
| | | | | | (discontinued) |
| Zidovudine | Retrovir ®, | GSK | 300 mg twice daily | Antiretroviral (HIV, AIDS) | FDA |
| | Retrovis | | | | approved |

*Typical adult dose or dose range for various indications.
AIDS = acquired immune deficiency syndrome.
ALL = acute lymphocytic leukemia.
AML = acute myelogenous leukemia.
BW = body weight.
CLL = chronic lymphocytic leukemia.
CML = chronic myelogenous leukemia.
CRC = colorectal cancer.
CTCL = cutaneous T-cell lymphoma.
INN = International Nonproprietary Name.
i.v. = intravenous administration.
GIST = gastrointestinal stromal tumor.
HCC = hepatocellular carcinoma.
HIV = human immunodeficiency virus.
MDS = myelodysplastic syndrome.
MTC = Medullary thyroid cancer.
NSCLC = Non-small-cell lung carcinoma.
p.o. = oral administration.
RCC = renal cell carcinoma.
SSL = small lymphocytic lymphoma.

TABLE 4
Representative examples of anticancer and antiviral agents and their chemical formula.

| NAME (INN) | Formula | Systematic (IUPAC) name |
|---|---|---|
| Abacavir | C14H18N6O | {(1S,4R)-4-[2-amino-6-(cyclopropylamino)-9H-purin-9- |
| | | yl]cyclopent-2-en-1-yl}methanol |
| Acyclovir | C8H11N5O3 | 2-Amino-9-(propoxymethyl)-1H-purin-6(9H)-one |
| Adefovir | C8H12N5O4P | {[2-(6-amino-9H-purin-9-yl)ethoxy]methyl}phosphonic acid |
| Amdoxovir | C9H12N6O3 | [(2R,4R)-4-(2,6-Diaminopurin-9-yl)-1,3-dioxolan-2- |
| | | yl]methanol |
| Apricitabine | C8H11N3O3S | 4-amino-1-[(2R,4R)-2-(hydroxymethyl)-1,3-oxathiolan-4- |
| | | yl]pyrimidin-2(1H)-one |
| Azacitidine | C8H12N4O5 | 4-amino-1-β-D-ribofuranosyl-1,3,5-triazin-2(1H)-one |
| Capecitabine | C15H22FN3O6 | pentyl[1-(3,4-dihydroxy-5-methyl-tetrahydrofuran-2-yl)-5- |
| | | fluoro-2-oxo-1H-pyrimidin-4-yl]aminomethanoate |
| Cladribine | C10H12ClN5O3 | 5-(6-amino-2-chloro-purin-9-yl)-2-(hydroxymethyl)oxolan- |
| (2CDA) | | 3-ol |
| Clevudine | C10H13FN2O5 | 1-[(2S,3R,4S,5S)-3-fluoro-4-hydroxy-5- |
| | | (hydroxymethyl)oxolan-2-yl]-5-methylpyrimidine-2,4-dione |
| Clofarabine | C10HClFN5O3 | 5-(6-amino-2-chloro-purin-9-yl)-4-fluoro-2- |
| | | (hydroxymethyl)oxolan-3-ol |
| Cytarabine (Ara-C) | C9H13N3O5 | 4-amino-1-[(2R,3S,4R,5R)-3,4-dihydroxy-5- |
| | | (hydroxymethyl)oxolan-2-yl] pyrimi |
| din-2-one | | |
| Decitabine | C8H12N4O4 | 4-amino-1-(2-deoxy-b-D-erythro-pentofuranosyl)- |
| | | 1,3,5-triazin-2(1H)-one |
| Didanosine | C10H12N4O3 | 9-[(2R,5S)-5-(hydroxymethyl)oxolan-2-yl]-6,9-dihydro-3H- |
| | | purin-6-one |
| Elvucitabine | C9H10FN3O3 | 4-Amino-5-fluoro-1-[(2S,5R)-5-(hydroxymethyl)-2,5- |
| | | dihydrofuran-2-yl]pyrimidin-2-one |
| Emtricitabine | C8H10FN3O3S | 4-amino-5-fluoro-1-[(2R,5S)-2-(hydroxymethyl)-1,3- |
| | | oxathiolan-5-yl]-1,2-dihydropyrimidin-2-one |
| Entecavir | C12H15N5O3 | 2-Amino-9-[(1S,3R,4S)-4-hydroxy-3-(hydroxymethyl)-2- |
| | | methylidenecyclopentyl]-6,9-dihydro-3H-purin-6-one |
| Festinavir | | |
| Fludarabine | C10H13FN5O7P | [(2R,3R,4S,5R)-5-(6-amino-2-fluoro-purin-9-yl)-3,4- |
| | | dihydroxy-oxolan-2-yl]methoxyphosphonic acid |
| Fluorouracil | C4H3FN2O2 | 5-fluoro-1H-pyrimidine-2,4-dione |
| Gemcitabine | C9H11F2N3O4 | 4-amino-1-(2-deoxy-2,2-difluoro-β-D-erythro- |
| | | pentofuranosyl)pyrimidin-2(1H)-on 2′,2′-difluoro-2′- |
| | | deoxycytidine |
| Idoxuridine | C9H11IN2O5 | 1-[(2R,4S,5R)-4-hydroxy-5-(hydroxymethyl)oxolan-2-yl]-5- |
| | | iodo-1,2,3,4-tetrahydropyrimidine-2,4-dione |
| KP-1461 | C8H14N4O4 | |
| Lamivudine | C8H11N3O3S | 4-amino-1-[(2R,5S)-2-(hydroxymethyl)-1,3-oxathiolan-5- |
| | | yl]-1,2-dihydropyrimidin-2-one |
| Nelarabine | C11H15N5O5 | (2R,3S,4R,5R)-2-(2-amino-6-methoxy-purin-9-yl)-5- |
| | | (hydroxymethyl)oxolane-3,4-diol |
| Racivir | C8H10FN3O3S | 4-Amino-5-fluoro-1-[(2S,5R)-2-(hydroxymethyl)-1,3- |
| | | oxathiolan-5-yl]pyrimidin-2(1H)-one |
| Ribavirin | | |
| Sapacitabine | C26H42N4O5 | 1-(2-cyano-2-deoxy-β-D-arabinofuranosyl)-4- |
| | | (palmitoylamino)pyrimidin-2(1H)-one |
| Stavudine | C10H12N2O4 | 1-((2R,5S)-5-(hydroxymethyl)-2,5-dihydrofuran-2-yl)-5- |
| | | methylpyrimidine-2,4(1H,3H)-dione |
| Taribavirin | C8H13N5O4 | 1-[(2R,3R,4S,5S)-3,4-dihydroxy-5-(hydroxymethyl)oxolan- |
| | | 2-yl]-1,2,4-triazole-3-carboximidamide |
| Telbivudine | C10H14N2O5 | 1-(2-deoxy-β-L-erythro-pentofuranosyl)-5- |
| | | methylpyrimidine-2,4(1H,3H)-dione |
| Tenofovir | C9H14N5O4P | ({[(2R)-1-(6-amino-9H-purin-9-yl)propan-2- |
| | | yl]oxy}methyl)phosphonic acid |
| Tezacitabine | C10H12FN3O4 | 4-amino-1-[(2R,3E,4S,5R)-3-(fluoromethylidene)-4- |
| | | hydroxy-5-(hydroxymethyl)oxolan-2-yl]pyrimidin-2-one |
| Trifluridine | C10H11F3N2O5 | 1-[4-hydroxy-5-(hydroxymethyl)oxolan-2-yl]-5- |
| | | (trifluoromethyl) pyrimidine-2,4-dione |
| Troxacitabine | C8H11N3O4 | 4-amino-1-[(2S,4S)-2-(hydroxymethyl)-1,3-dioxolan-4- |
| | | yl]pyrimidin-2(1H)-one |
| Vidarabine | C10H15N5O5 | (2R,3S,4S,5R)-2-(6-amino-9H-purin-9-yl)-5- |
| | | (hydroxymethyl)oxolane-3,4-diol hydrate |
| Zalcitabine | C9H13N3O3 | 4-amino-1-((2R,5S)-5-(hydroxymethyl)tetrahydrofuran-2- |
| | | yl)pyrimidin-2(1H)-one |
| Zidovudine | C10H13N5O4 | 1-[(2R,4S,5S)-4-azido-5-(hydroxymethyl)oxolan-2-yl]-5- |
| | | methylpyrimidine-2,4-dione |

In one preferred embodiment of the above-depicted treatment, wherein the patient is under treatment or is to be treated with one or more anticancer or antiviral agent, for example, (deoxy)nucleotide or (deoxy)nucleoside analog drugs, and is not refractory or resistant to said anticancer or antiviral agent(s), the small molecule inhibitor(s) (for example, ATP competitive inhibitors, signal transduction inhibitors/activators, protein kinase inhibitors/activators, tyrosine kinase inhibitors/activators, and especially masitinib or a pharmaceutically acceptable salt or hydrate thereof), to be administered in combination with said (deoxy)nucleotide or (deoxy)nucleoside analog drug(s), is dosed ideally in accordance to the manufacture's recommendations, with the (deoxy)nucleotide or (deoxy)nucleoside analog drug(s) dosed in accordance to the manufacture's recommendations or some numeric fraction less than the manufacture's recommendations. The magnitude of this numeric fraction depends on the degree of synergy or sensitization between a given combination of small molecule inhibitor(s)/activator(s) and (deoxy)nucleotide or (deoxy)nucleoside analog drug(s), and also on the type of cancer (including hematological malignancies) or viral infection being treated. To a first approximation, this numeric fraction, or ‘analog-sparing/sensitization factor’, can be estimated as the reciprocal of the half inhibitory concentration (IC₅₀) (that is to say, a dose for a given therapeutic effect) of the (deoxy)nucleotide or (deoxy)nucleoside analog agent(s) alone divided by the equivalent IC₅₀ (or dose for said given therapeutic effect) when in combination with the small molecule inhibitor(s)/activator(s), dosed ideally in accordance to the manufacture's recommendations.

In the example of the analog-sparing/sensitization factor being equal to 0.5, the (deoxy)nucleotide or (deoxy)nucleoside analog treatment step would require approximately half (50%) the manufacture's recommended dose to achieve the equivalent therapeutic effect, with the small molecule inhibitor/activator treatment step being dosed in accordance to the manufacture's recommendations. In the example of the analog-sparing/sensitization factor being equal to 0.1, the (deoxy)nucleotide or (deoxy)nucleoside analog treatment step would require approximately one tenth (10%) the manufacture's recommended dose to achieve the equivalent therapeutic effect, with the small molecule inhibitor/activator treatment step being dosed in accordance to the manufacture's recommendations. In the example of the analog-sparing/sensitization factor being equal to 0.05, the (deoxy)nucleotide or (deoxy)nucleoside analog treatment step would require approximately one twentieth (5%) the manufacture's recommended dose to achieve the equivalent therapeutic effect, with the small molecule inhibitor/activator treatment step being dosed in accordance to the manufacture's recommendations.

To further exemplify the present invention's concept of small molecule inhibitor/activator induced analog-sparing and analog-sensitization treatment regimens, consider the manufacture's recommended dose of the small molecule inhibitor/activator masitinib (at least 6.0 mg±1.5 mg/kg/day over a 28 day cycle), and that of the nucleoside analog gemcitabine (1000±250 mg/m² of patient surface area weekly for 3 weeks followed by 1 week of rest, every 28 days). It follows that a hypothetical analog-sparing/sensitization factor of 0.5, 0.1, or 0.05 would allow for a reduction in gemcitabine dose to 500, 100, or 50 mg/m², respectively. Alternatively, if gemcitabine is dosed at the manufacture's recommended dose as part of a small molecule inhibitor/activator combination therapy with a hypothetical analog-sparing/sensitization factor of 0.8, 0.66, or 0.5, the therapeutic effect would be equivalent to that achieved from a gemcitabine dose of 1250, 1500, or 2000 mg/m², respectively; however, with approximately the same toxicity associated with the manufacture's recommended dose.

Within this framework of analog-sparing or analog-sensitization regimens, many dosing combinations exist that will achieve the equivalent therapeutic effect; that is to say, the (deoxy)nucleotide or (deoxy)nucleoside analog treatment step may administer a dose within a range from the manufacture's recommended dose for single agent use, representing the maximum (deoxy)nucleotide or (deoxy)nucleoside analog dose, to the minimum analog-sparing dose when administered in combination with small molecule inhibitor/activator treatment step, said small molecule inhibitor(s)/activator(s) dosed in accordance to the manufacture's recommendations. In the situation where all other parameters are stable, as the dose of the (deoxy)nucleotide or (deoxy)nucleoside analog treatment step varies, the dose of the small molecule inhibitor/activator treatment step would need to counterbalance that change to maintain a stable therapeutic effect. For example, an increased (deoxy)nucleotide or (deoxy)nucleoside analog dose would require a decrease in small molecule inhibitor/activator dose to maintain a constant therapeutic effect. In practice, dosing combinations between the (deoxy)nucleotide or (deoxy)nucleoside analog treatment step and small molecule treatment step can be a considered a dynamic process that needs to be tailored to the individual patient in order to optimize the balance between response and toxicity throughout treatment, both of which are likely to vary over time and duration of drug exposure depending upon adverse reactions of the possible drug combination, changes in patient tolerance to adverse effects, and the patient's susceptibility of developing resistance to the (deoxy)nucleotide or (deoxy)nucleoside analog drug(s).

The combination therapy can provide a therapeutic advantage in view of the dissimilar toxicity associated with the individual treatment modalities used. For example, treatment with small molecule inhibitors/activators can lead to a particular toxicity that is not seen with anticancer or antiviral agents, and vice versa. When the therapeutic effect achieved is the result of the combination treatment producing an enhanced or synergistic effect, the doses of each agent can be administered at a dose for which said toxicities do not exist or are minimal, such that together the combination therapy provides a therapeutic dose while avoiding the toxicities of each of the constituents of the combination agents.

In another preferred embodiment of the above-depicted treatment, wherein the patient is refractory or resistant to the anticancer or antiviral agent, for example, (deoxy)nucleotide or (deoxy)nucleoside analogs, the administered (deoxy)nucleotide or (deoxy)nucleoside analog drug(s) is dosed ideally in accordance to the manufacture's recommendations, with the small molecule inhibitor's)/activator(s) to be administered in combination also dosed ideally in accordance to the manufacture's recommendations. In this regard, the small molecule inhibitor/activator, especially masitinib or a pharmaceutically acceptable salt or hydrate thereof, and at least one anticancer or antiviral agent, especially (deoxy)nucleotide or (deoxy)nucleoside analog drug, are to be administered separately, simultaneously or sequentially in time.

Since there is no established mechanism of resistance, not all patients may express a dCK-associated drug resistance. In one particular embodiment, the present invention relates to a method for treating cancer (including hematological malignancies) or viral infections, wherein said treatment comprises administering at least one small molecule inhibitor/activator (including ATP competitive inhibitors, signal transduction inhibitors/activators, protein kinase inhibitors/activators, and tyrosine kinase inhibitors/activators, and especially masitinib or a pharmaceutically acceptable salt or hydrate thereof), to a patient or group of patients with an under-expression, down-regulation, or decreased activity of dCK. Optionally, said method comprises a step of identifying an under-expression, down-regulation, or decreased activity of dCK. In particular, said method comprises administering to said patient or group of patients at least another anticancer or antiviral agent, different from said small molecule inhibitor/activator.

The identification of patients with an under-expression, down-regulation, or decreased activity of dCK can be made using methods previously described, including but not limited to: real-time quantitative PCR [Mansson E, et al. Leukemia (2002) 16, 386]; or immunocytochemistry [Hubeek I, et al. J Clin Pathol 2005; 58:695]; or [18F]fluorodeoxyglucos positron emission tomography (PET) [Laing R, et al. Proc Natl Acad Sci USA. 2009; 106(8):2847]. For example, immunocytochemistry is an effective and reliable method for determining the expression of dCK in patient samples and requires little tumour material. This method enables large scale screening of dCK expression in tumour samples.

In the absence of drug resistance, the main clinical limitation on use of (deoxy)nucleotide and (deoxy)nucleoside analogs at their standard dosage regimen is high toxicity in healthy tissues, with subsequent life-threatening adverse events or lower patient quality of life and poorer treatment compliance and lower drug exposure. The identification of patients with intolerance to the standard dosage regimen of (deoxy)nucleotide and (deoxy)nucleoside analogs is made through patient safety assessment on occurrence of adverse events, as defined by the Medical Dictionary for Regulatory Activities (MedDRA) coding and adverse event classification dictionary, or the Common Terminology Criteria for Adverse Events (CTCAE). An adverse event is defined as any modification of the clinical status of the patient, i.e. any emergence of a disease, sign or symptom, or modification of sign, symptom or concomitant disease, regardless of its relationship to study medication.

In one particular embodiment, the present invention relates to a method for treating cancer (including hematological malignancies) or viral infections, wherein said treatment comprises administering at least one small molecule inhibitor/activator (including ATP competitive inhibitors, signal transduction inhibitors/activators, protein kinase inhibitors/activators, and tyrosine kinase inhibitors/activators, and especially masitinib or a pharmaceutically acceptable salt or hydrate thereof), to a patient or group of patients who are intolerant to the standard dosage regimen of at least another anticancer or antiviral agent, different from said small molecule inhibitor/activator.

In one embodiment of the present invention, at least one small molecule inhibitor/activator (including ATP competitive inhibitors, signal transduction inhibitors/activators, protein kinase inhibitors/activators, and tyrosine kinase inhibitors/activators, and especially masitinib or a pharmaceutically acceptable salt or hydrate thereof), can be administered for the treatment of cancer (including hematological malignancies) or viral infections in combination with, and without particular limitation, at least one of the following anticancer or antiviral agents: abacavir, acyclovir, adefovir, amdoxovir, apricitabine, Atripla®, azacitidine, capecitabine, cladribine, movectro, clevudine, clofarabine, evoltra, Combivir®, cytarabine, decitabine, didanosine, elvucitabine, emtricitabine, entecavir, Epzicom®, festinavir, fludarabine, fluorouracil, gemcitabine, idoxuridine, KP-1461, lamivudine, nelarabine, racivir, ribavirin, sapacitabine, stavudine, taribavirin, telbivudine, tenofovir, tezacitabine, trifluridine, Trizivir®, troxacitabine, Truvada®, vidarabine, zalcitabine, or zidovudine (see Table 3 and 4 for chemical and structural formulae, dosing and manufacturing details).

In another embodiment of the present invention, said small molecule inhibitor/activator is administered in combination with azacitidine as part of an anticancer treatment. A particular example would be a product consisting of azacitidine and masitinib (or a pharmaceutically acceptable salt or hydrate thereof) used for the treatment of myelodysplastic syndromes.

In another embodiment of the present invention, said small molecule inhibitor/activator is administered in combination with capecitabine as part of an anticancer treatment. A particular example would be a product consisting of capecitabine and masitinib (or a pharmaceutically acceptable salt or hydrate thereof) used for the treatment of colon cancer. Another example would be a product consisting of capecitabine and masitinib (or a pharmaceutically acceptable salt or hydrate thereof) used for the treatment of metastasized breast cancer.

In another embodiment of the present invention, said small molecule inhibitor/activator is administered in combination with cladribine as part of an anticancer treatment. A particular example would be a product consisting of cladribine and masitinib (or a pharmaceutically acceptable salt or hydrate thereof) used for the treatment of hairy cell leukemia. Another example would be a product consisting of cladribine and masitinib (or a pharmaceutically acceptable salt or hydrate thereof) used for the treatment of systemic mastocytosis. Yet another example would be a product consisting of cladribine and masitinib (or a pharmaceutically acceptable salt or hydrate thereof) used for the treatment of multiple sclerosis.

In another embodiment of the present invention, said small molecule inhibitor/activator is administered in combination with clofarabine as part of an anticancer treatment. A particular example would be a product consisting of clofarabine and masitinib (or a pharmaceutically acceptable salt or hydrate thereof) used for the treatment of acute lymphoblastic leukemia.

In another embodiment of the present invention, said small molecule inhibitor/activator is administered in combination with cytarabine as part of an anticancer treatment. A particular example would be a product consisting of cytarabine and masitinib (or a pharmaceutically acceptable salt or hydrate thereof) used for the treatment of acute lymphoblastic leukemia. Another example would be a product consisting of cytarabine and masitinib (or a pharmaceutically acceptable salt or hydrate thereof) used for the treatment of chronic myelogenous leukemia. Yet another example would be a product consisting of cytarabine and masitinib (or a pharmaceutically acceptable salt or hydrate thereof) used for the treatment of acute myeloid leukemia.

In another embodiment of the present invention, said small molecule inhibitor/activator is administered in combination with decitabine as part of an anticancer treatment. A particular example would be a product consisting of decitabine and masitinib (or a pharmaceutically acceptable salt or hydrate thereof) used for the treatment of myelodysplastic syndromes.

In another embodiment of the present invention, said small molecule inhibitor/activator is administered in combination with fludarabine as part of an anticancer treatment. A particular example would be a product consisting of fludarabine and masitinib (or a pharmaceutically acceptable salt or hydrate thereof) used for the treatment of chronic lymphocytic leukemia.

In another embodiment of the present invention, said small molecule inhibitor/activator is administered in combination with fluorouracil as part of an anticancer treatment. A particular example would be a product consisting of fluorouracil and masitinib (or a pharmaceutically acceptable salt or hydrate thereof) used for the treatment of pancreatic cancer. Another example would be a product consisting of fluorouracil and masitinib (or a pharmaceutically acceptable salt or hydrate thereof) used for the treatment of breast cancer. Another example would be a product consisting of fluorouracil and masitinib (or a pharmaceutically acceptable salt or hydrate thereof) used for the treatment of actinic keratosis. Another example would be a product consisting of fluorouracil and masitinib (or a pharmaceutically acceptable salt or hydrate thereof) used for the treatment of advanced colorectal cancer. Another example would be a product consisting of fluorouracil and masitinib (or a pharmaceutically acceptable salt or hydrate thereof) used for the treatment of basal cell carcinoma. Another example would be a product consisting of fluorouracil and masitinib (or a pharmaceutically acceptable salt or hydrate thereof) used for the treatment of gastricadenocarcinoma. Another example would be a product consisting of fluorouracil and masitinib (or a pharmaceutically acceptable salt or hydrate thereof) used for the treatment of squamous cell carcinoma of the head and neck. Another example would be a product consisting of fluorouracil and masitinib (or a pharmaceutically acceptable salt or hydrate thereof) used for the treatment of stomach cancer.

In another embodiment of the present invention, said small molecule inhibitor/activator is administered in combination with gemcitabine as part of an anticancer treatment. A particular example would be a product consisting of gemcitabine and masitinib (or a pharmaceutically acceptable salt or hydrate thereof) used for the treatment of advanced or metastatic pancreatic cancer. Another example would be a product consisting of gemcitabine and masitinib (or a pharmaceutically acceptable salt or hydrate thereof) used for the treatment of breast cancer that has metastasized. Another example would be a product consisting of gemcitabine and masitinib, or a pharmaceutically acceptable salt or hydrate thereof, in the treatment advanced or metastatic non-small cell lung cancer. Another example would be a product consisting of gemcitabine and masitinib (or a pharmaceutically acceptable salt or hydrate thereof) used for the treatment of advanced or metastatic ovarian cancer. Another example would be a product consisting of gemcitabine and masitinib (or a pharmaceutically acceptable salt or hydrate thereof) used for the treatment of biliary tract cancer. Another example would be a product consisting of gemcitabine and masitinib (or a pharmaceutically acceptable salt or hydrate thereof) used for the treatment of bladder cancer. Another example would be a product consisting of gemcitabine and masitinib (or a pharmaceutically acceptable salt or hydrate thereof) used for the treatment of cervical cancer. Another example would be a product consisting of gemcitabine and masitinib (or a pharmaceutically acceptable salt or hydrate thereof) used for the treatment of malignant mesothelioma.

In another embodiment of the present invention, said small molecule inhibitor/activator is administered in combination with nelarabine as part of an anticancer treatment. A particular example would be a product consisting of nelarabine and masitinib (or a pharmaceutically acceptable salt or hydrate thereof) used for the treatment of T-cell acute lymphoblastic leukemia. Another example would be a product consisting of nelarabine and masitinib (or a pharmaceutically acceptable salt or hydrate thereof) used for the treatment of T-cell lymphoblastic lymphoma.

In another embodiment of the present invention, said small molecule inhibitor/activator is administered in combination with sapacitabine as part of an anticancer treatment. A particular example would be a product consisting of sapacitabine and masitinib (or a pharmaceutically acceptable salt or hydrate thereof) used for the treatment of acute myeloid leukemia. Another example would be a product consisting of sapacitabine and masitinib (or a pharmaceutically acceptable salt or hydrate thereof) used for the treatment of myelodysplastic syndromes.

In another embodiment of the present invention, said small molecule inhibitor/activator is administered in combination with tezacitabine as part of an anticancer treatment. A particular example would be a product consisting of tezacitabine and masitinib (or a pharmaceutically acceptable salt or hydrate thereof) used for the treatment of solid tumors.

In another embodiment of the present invention, said small molecule inhibitor/activator is administered in combination with troxacitabine as part of an anticancer treatment. A particular example would be a product consisting of troxacitabine and masitinib (or a pharmaceutically acceptable salt or hydrate thereof) used for the treatment of acute myeloid leukemia.

In another embodiment of the present invention, said small molecule inhibitor/activator is administered in combination with abacavir as part of an antiviral treatment. A particular example would be a product consisting of abacavir and masitinib (or a pharmaceutically acceptable salt or hydrate thereof) used for the treatment of HIV.

In another embodiment of the present invention, said small molecule inhibitor/activator is administered in combination with acyclovir as part of an antiviral treatment. A particular example would be a product consisting of acyclovir and masitinib (or a pharmaceutically acceptable salt or hydrate thereof) used for the treatment of herpes viruses.

In another embodiment of the present invention, said small molecule inhibitor/activator is administered in combination with adefovir as part of an antiviral treatment. A particular example would be a product consisting of adefovir and masitinib (or a pharmaceutically acceptable salt or hydrate thereof) used for the treatment of hepatitis B.

In another embodiment of the present invention, said small molecule inhibitor/activator is administered in combination with amdoxovir as part of an antiviral treatment. A particular example would be a product consisting of amdoxovir and masitinib (or a pharmaceutically acceptable salt or hydrate thereof) used for the treatment of HIV.

In another embodiment of the present invention, said small molecule inhibitor/activator is administered in combination with apricitabine as part of an antiviral treatment. A particular example would be a product consisting of apricitabine and masitinib (or a pharmaceutically acceptable salt or hydrate thereof) used for the treatment of HIV.

In another embodiment of the present invention, said small molecule inhibitor/activator is administered in combination with Atripla® as part of an antiviral treatment. A particular example would be a product consisting of Atripla® and masitinib (or a pharmaceutically acceptable salt or hydrate thereof) used for the treatment of HIV.

In another embodiment of the present invention, said small molecule inhibitor/activator is administered in combination with clevudine as part of an antiviral treatment. A particular example would be a product consisting of clevudine and masitinib (or a pharmaceutically acceptable salt or hydrate thereof) used for the treatment of hepatitis B.

In another embodiment of the present invention, said small molecule inhibitor/activator is administered in combination with Combivir® as part of an antiviral treatment. A particular example would be a product consisting of Combivir® and masitinib (or a pharmaceutically acceptable salt or hydrate thereof) used for the treatment of HIV.

In another embodiment of the present invention, said small molecule inhibitor/activator is administered in combination with didanosine as part of an antiviral treatment. A particular example would be a product consisting of didanosine and masitinib (or a pharmaceutically acceptable salt or hydrate thereof) used for the treatment of HIV.

In another embodiment of the present invention, said small molecule inhibitor/activator is administered in combination with elvucitabine as part of an antiviral treatment. A particular example would be a product consisting of elvucitabine and masitinib (or a pharmaceutically acceptable salt or hydrate thereof) used for the treatment of HIV.

In another embodiment of the present invention, said small molecule inhibitor/activator is administered in combination with emtricitabine as part of an antiviral treatment. A particular example would be a product consisting of emtricitabine and masitinib (or a pharmaceutically acceptable salt or hydrate thereof) used for the treatment of HIV. Another example would be a product consisting of emtricitabine and masitinib (or a pharmaceutically acceptable salt or hydrate thereof) used for the treatment of hepatitis B.

In another embodiment of the present invention, said small molecule inhibitor/activator is administered in combination with entecavir as part of an antiviral treatment. A particular example would be a product consisting of entecavir and masitinib (or a pharmaceutically acceptable salt or hydrate thereof) used for the treatment of hepatitis B.

In another embodiment of the present invention, said small molecule inhibitor/activator is administered in combination with Epzicom® as part of an antiviral treatment. A particular example would be a product consisting of Epzicom® and masitinib (or a pharmaceutically acceptable salt or hydrate thereof) used for the treatment of HIV.

In another embodiment of the present invention, said small molecule inhibitor/activator is administered in combination with festinavir as part of an antiviral treatment. A particular example would be a product consisting of festinavir and masitinib (or a pharmaceutically acceptable salt or hydrate thereof) used for the treatment of HIV.

In another embodiment of the present invention, said small molecule inhibitor/activator is administered in combination with idoxuridine as part of an antiviral treatment. A particular example would be a product consisting of idoxuridine and masitinib (or a pharmaceutically acceptable salt or hydrate thereof) used for the treatment of herpes viruses.

In another embodiment of the present invention, said small molecule inhibitor/activator is administered in combination with KP-1461 as part of an antiviral treatment. A particular example would be a product consisting of KP-1461 and masitinib (or a pharmaceutically acceptable salt or hydrate thereof) used for the treatment of HIV.

In another embodiment of the present invention, said small molecule inhibitor/activator is administered in combination with lamivudine as part of an antiviral treatment. A particular example would be a product consisting of lamivudine and masitinib (or a pharmaceutically acceptable salt or hydrate thereof) used for the treatment of HIV. Another example would be a product consisting of lamivudine and masitinib (or a pharmaceutically acceptable salt or hydrate thereof) used for the treatment of hepatitis B.

In another embodiment of the present invention, said small molecule inhibitor/activator is administered in combination with racivir as part of an antiviral treatment. A particular example would be a product consisting of racivir and masitinib (or a pharmaceutically acceptable salt or hydrate thereof) used for the treatment of HIV.

In another embodiment of the present invention, said small molecule inhibitor/activator is administered in combination with ribavirin as part of an antiviral treatment. A particular example would be a product consisting of ribavirin and masitinib (or a pharmaceutically acceptable salt or hydrate thereof) used for the treatment of hepatitis C.

In another embodiment of the present invention, said small molecule inhibitor/activator is administered in combination with stavudine as part of an antiviral treatment. A particular example would be a product consisting of stavudine and masitinib (or a pharmaceutically acceptable salt or hydrate thereof) used for the treatment of HIV.

In another embodiment of the present invention, said small molecule inhibitor/activator is administered in combination with taribavirin as part of an antiviral treatment. A particular example would be a product consisting of taribavirin and masitinib (or a pharmaceutically acceptable salt or hydrate thereof) used for the treatment of hepatitis C.

In another embodiment of the present invention, said small molecule inhibitor/activator is administered in combination with telbivudine as part of an antiviral treatment. A particular example would be a product consisting of telbivudine and masitinib (or a pharmaceutically acceptable salt or hydrate thereof) used for the treatment of hepatitis B.

In another embodiment of the present invention, said small molecule inhibitor/activator is administered in combination with tenofovir as part of an antiviral treatment. A particular example would be a product consisting of tenofovir and masitinib (or a pharmaceutically acceptable salt or hydrate thereof) used for the treatment of HIV.

In another embodiment of the present invention, said small molecule inhibitor/activator is administered in combination with trifluridine as part of an antiviral treatment. A particular example would be a product consisting of trifluridine and masitinib (or a pharmaceutically acceptable salt or hydrate thereof) used for the treatment of herpes viruses.

In another embodiment of the present invention, said small molecule inhibitor/activator is administered in combination with Trizivir® as part of an antiviral treatment. A particular example would be a product consisting of Trizivir® and masitinib (or a pharmaceutically acceptable salt or hydrate thereof) used for the treatment of HIV.

In another embodiment of the present invention, said small molecule inhibitor/activator is administered in combination with Truvada® as part of an antiviral treatment. A particular example would be a product consisting of Truvada® and masitinib (or a pharmaceutically acceptable salt or hydrate thereof) used for the treatment of HIV.

In another embodiment of the present invention, said small molecule inhibitor/activator is administered in combination with vidarabine as part of an antiviral treatment. A particular example would be a product consisting of vidarabine and masitinib (or a pharmaceutically acceptable salt or hydrate thereof) used for the treatment of herpes viruses.

In another embodiment of the present invention, said small molecule inhibitor/activator is administered in combination with zalcitabine as part of an antiviral treatment. A particular example would be a product consisting of zalcitabine and masitinib (or a pharmaceutically acceptable salt or hydrate thereof) used for the treatment of HIV.

In another embodiment of the present invention, said small molecule inhibitor/activator is administered in combination with zidovudine as part of an antiviral treatment. A particular example would be a product consisting of zidovudine and masitinib (or a pharmaceutically acceptable salt or hydrate thereof) used for the treatment of HIV.

In one embodiment of the above-depicted treatment, the small molecule inhibitor/activator is administered in the form of a mesilate; the orally bioavailable mesylate salt of the small molecule inhibitor/activator.

For example, in one preferred embodiment of the above-depicted treatment, the small molecule inhibitor/activator is masitinib, administered in the form of masitinib mesilate; the orally bioavailable mesylate salt of masitinib—CAS 1048007-93-7 (MsOH); C28H30N6OS.CH3SO3H; MW 594.76. Depending on age, individual condition, mode of administration, and the clinical setting, effective doses of masitinib or a pharmaceutically acceptable salt or hydrate thereof in human patients are 3.0 to 12.0 mg/kg/day per os, preferably in two daily intakes. Given that the masitinib dose in mg/kg/day used in the described dose regimens refers to the amount of active ingredient masitinib, compositional variations of a pharmaceutically acceptable salt of masitinib mesilate will not change the said dose regimens.

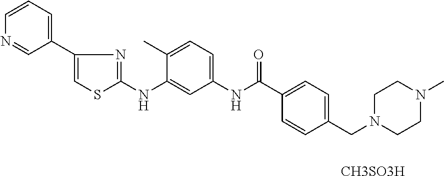

Pharmaceutically acceptable salts are pharmaceutically acceptable acid addition salts, like for example with inorganic acids, such as hydrochloric acid, sulfuric acid or a phosphoric acid, or with suitable organic carboxylic or sulfonic acids, for example aliphatic mono- or di-carboxylic acids, such as trifluoroacetic acid, acetic acid, propionic acid, glycolic acid, succinic acid, maleic acid, fumaric acid, hydroxymaleic acid, malic acid, tartaric acid, citric acid or oxalic acid, or amino acids such as arginine or lysine, aromatic carboxylic acids, such as benzoic acid, 2-phenoxy-benzoic acid, 2-acetoxy-benzoic acid, salicylic acid, 4-aminosalicylic acid, aromatic-aliphatic carboxylic acids, such as mandelic acid or cinnamic acid, heteroaromatic carboxylic acids, such as nicotinic acid or isonicotinic acid, aliphatic sulfonic acids, such as methane-, ethane- or 2-hydroxyethane-sulfonic, in particular methanesulfonic acid (or mesilate), or aromatic sulfonic acids, for example benzene-, p-toluene- or naphthalene-2-sulfonic acid.

The small molecule inhibitor/activator can be administered by any known administration method known to a person skilled in the art. As is known to the person skilled in the art, various forms of excipients can be used adapted to the mode of administration and some of them can promote the effectiveness of the active molecule, e.g. by promoting a release profile rendering this active molecule overall more effective for the treatment desired. The pharmaceutical compositions of the invention are thus able to be administered in various forms. Examples of routes of administration include but are not limited to: an injectable, pulverizable or ingestible form, for example via the intramuscular, intravenous, subcutaneous, intradermal, oral, topical, rectal, vaginal, ophthalmic, nasal, transdermal or parenteral route. A preferred route is oral administration. The present invention notably covers the use of a compound according to the present invention for the manufacture of pharmaceutical composition.

According to a particular embodiment, the composition of the invention is an oral composition.

Such medicament can take the form of a pharmaceutical composition adapted for oral administration, which can be formulated using pharmaceutically acceptable carriers well known in the art in suitable dosages. Such carriers enable the pharmaceutical compositions to be formulated as tablets, pills, dragees, capsules, liquids, gels, syrups, slurries, suspensions, and the like, for ingestion by the patient. In addition to the active ingredients, these pharmaceutical compositions may contain suitable pharmaceutically-acceptable carriers comprising excipients and auxiliaries which facilitate processing of the active compounds into preparations which can be used pharmaceutically. Further details on techniques for formulation and administration may be found in the latest edition of Remington's Pharmaceutical Sciences (Maack Publishing Co., Easton, Pa.). The present inventions also covers a single pharmaceutical packaging comprising a small molecule inhibitor/activator, especially masitinib or a pharmaceutically acceptable salt thereof and at least one anticancer or antiviral agent, especially (deoxy)nucleotide or (deoxy)nucleoside analog drugs, including notably: gemcitabine, abacavir, acyclovir, adefovir, amdoxovir, apricitabine, azacitidine, Atripla®, capecitabine, cladribine, movectro, clevudine, clofarabine, evoltra, Combivir®, cytarabine, decitabine, didanosine, elvucitabine, emtricitabine, entecavir, Epzicom®, festinavir, fludarabine, fluorouracil, idoxuridine, KP-1461, lamivudine, nelarabine, racivir, ribavirin, sapacitabine, stavudine, taribavirin, telbivudine, tenofovir, tezacitabine, trifluridine, Trizivir®, troxacitabine, Truvada®, vidarabine, zalcitabine, or zidovudine.

It should be apparent to a person skilled in the art that the various modes of administration, dosages and dosing schedules described herein merely set forth specific embodiments and should not be construed as limiting the broad scope of the invention. Any permutations, variations and combinations of the dosages and dosing schedules are included within the scope of the present invention. Moreover, the specific dosage and dosage schedule of the anticancer or antiviral agent, especially (deoxy)nucleotide or (deoxy)nucleoside analog drugs, can vary, and the optimal dose, dosing schedule and route of administration will be determined based upon the specific anticancer of antiviral agent that is being used, mode of administration, patient status and condition, clinical setting, and cancer or viral infection being treated.

The route of administration of the small molecule inhibitors/activators is independent of the route of administration of the anticancer or antiviral agents. In an embodiment, the administration of the small molecule inhibitor/activator is oral administration. In another embodiment, the administration for the small molecule inhibitor/activator is intravenous administration. Thus, in accordance with these embodiments, the small molecule inhibitor/activator is administered orally or intravenously, and the anticancer or antiviral agent can be administered orally, parenterally, intraperitoneally, intravenously, intra-arterially, transdermally, sublingually, intramuscularly, rectally, transbuccally, intranasally, liposomally, via inhalation, vaginally, intraoccularly, via local delivery by catheter or stent, subcutaneously, intra-adiposally, intra-articularly, intrathecally, or in a slow release dosage form.

In addition, the small molecule inhibitor/activator and anticancer or antiviral agent may be administered by the same mode of administration, i.e. both agents administered e.g. orally, or intravenously. However, it is also within the scope of the present invention to administer the small molecule inhibitor/activator by one mode of administration, e.g. oral, and to administer the anticancer or antiviral agent by another mode of administration, e.g. intravenously or any other ones of the administration modes described hereinabove.

The compound(s) of the invention and one or more anticancer or antiviral agent, may be administered separately, simultaneously or sequentially in time. In one embodiment of the above-depicted treatment, the small molecule inhibitor/activator is administered as an adjuvant therapy following surgery, radiotherapy, or systemic therapy such as (deoxy)nucleotide or (deoxy)nucleoside analog drugs. In another embodiment of the present invention, the small molecule inhibitor/activator is administered as a neoadjuvant therapy prior to surgery, radiotherapy, or systemic therapy such as (deoxy)nucleotide or (deoxy)nucleoside analog drugs. In yet another embodiment of the present invention, the small molecule inhibitor/activator is administered as a concomitant or concurrent therapy, for example in combination with (deoxy)nucleotide or (deoxy)nucleoside analog drugs.

The present invention also relates to a method for combining at least two drugs for treating a cancer (including hematological malignancies) or a viral infection, optionally with a drug resistance, wherein said method comprises selecting among anticancer or antiviral agents a first drug that involves deoxynucleotide or deoxynucleoside kinase in its activation pathway, and in particular dCK, and administering to a patient said first drug in combination with at least one small molecule inhibitor/activator with dCK-modulating activity (including ATP competitive inhibitors, signal transduction inhibitors/activators, protein kinase inhibitors/activators, and tyrosine kinase inhibitors/activators, and especially masitinib or a pharmaceutically acceptable salt or hydrate thereof). In one embodiment said patient presents an under-expression, down-regulation, or decreased activity of dCK. In another embodiment said patient is intolerant to the standard dosage regimen of said anticancer or antiviral agent.

In the Drawings:

FIG. 1: Western blot analysis showing interaction between dCK and masitinib.

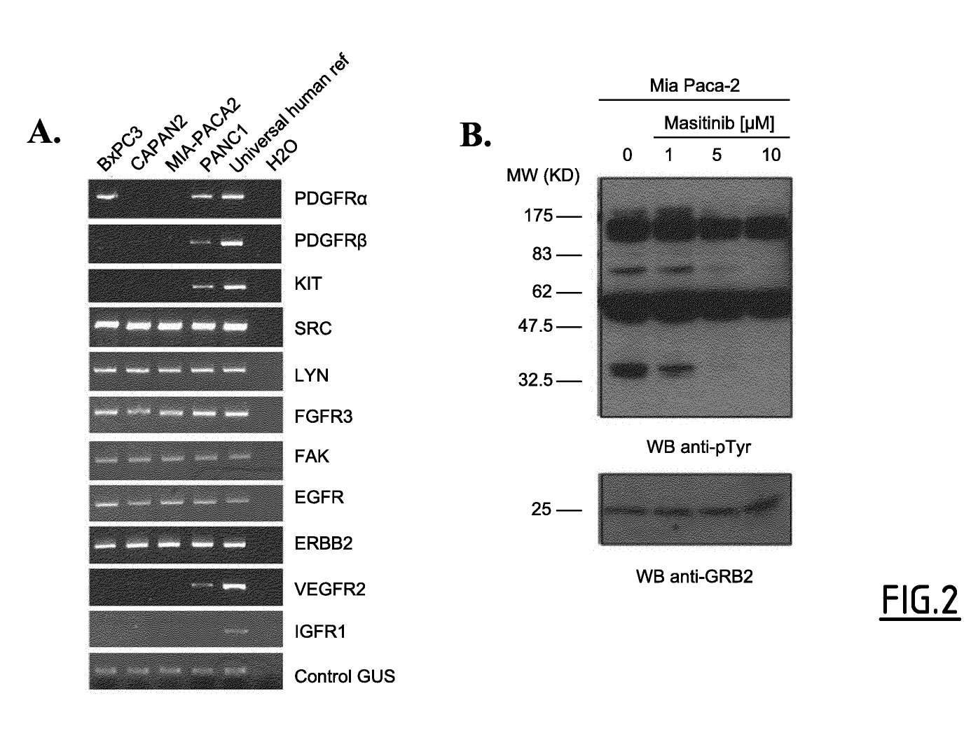

FIG. 2: Tyrosine kinase mRNA expression profile in human pancreatic cancer cell lines. (A) Messenger RNA expression of various receptor and cytoplasmic tyrosine kinases was analyzed by RT-PCR. Universal human reference total RNA was used as positive control for primers and the ubiquitous β-glucoronidase (GUS) served as an internal control for all RT-PCR reactions. (B) Tyrosine phosphorylation of proteins in response to masitinib. Mia Paca-2 cells (5×10⁶) were treated for 6 hours at 37° C. with various corcentrations of masitinib. Total cell lysates were prepared and tyrosine phosphorylation was analyzed by western blot with antibodies against phosphotyrosine (anti-pTyr). Anti-GRB2 WB demonstrates comparable loading of proteins. MW=molecular weight.

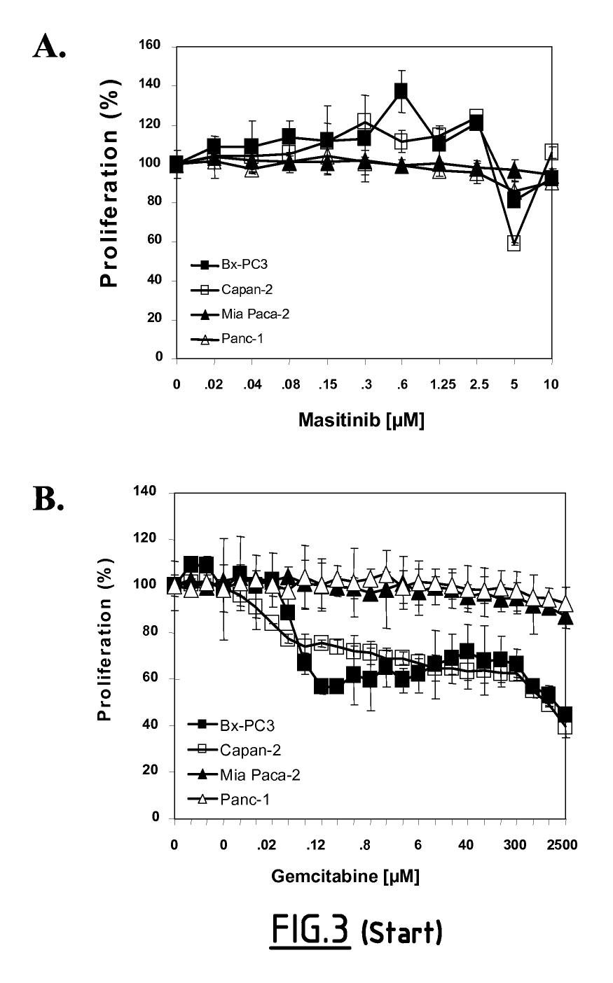

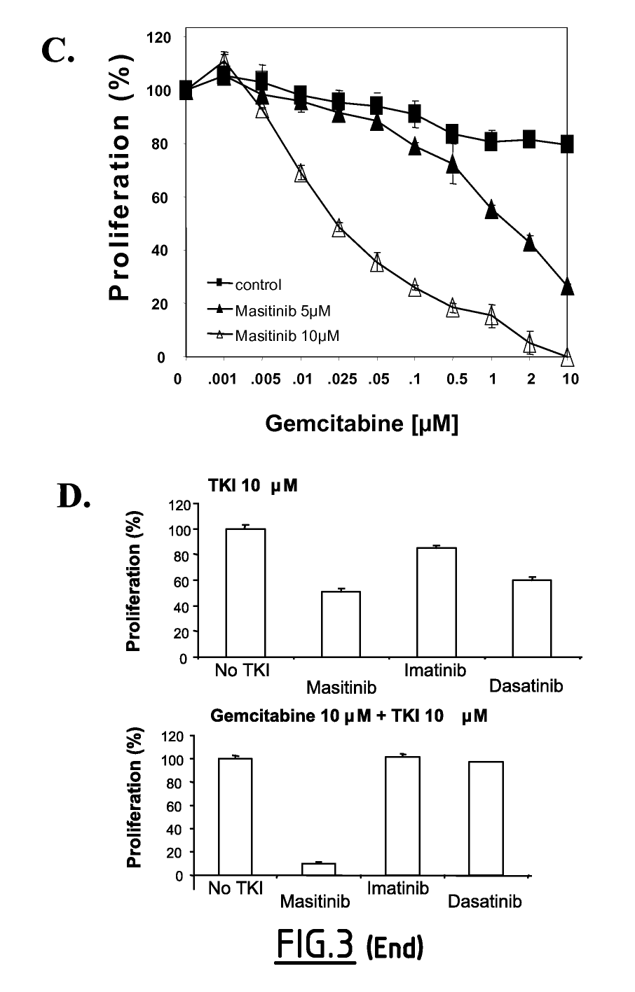

FIG. 3: Masitinib resensitization of resistant pancreatic tumor cell lines Mia Paca-2 and Panc-1 to gemcitabine. Sensitivity of pancreatic tumor cell lines to masitinib or gemcitabine as single agents, or in combination, was assessed using WST-1 proliferation assays. Four cell lines were tested for their sensitivity to masitinib (A) or gemcitabine (B). (C) Combination treatment of masitinib plus gemcitabine tested on gemcitabine resistant Mia Paca-2 cells. (D) Sensitivity of resistant Mia Paca-2 cells to various tyrosine kinase inhibitors alone (top) or in combination with gemcitabine (bottom) was analyzed in WST-1 proliferation assays.

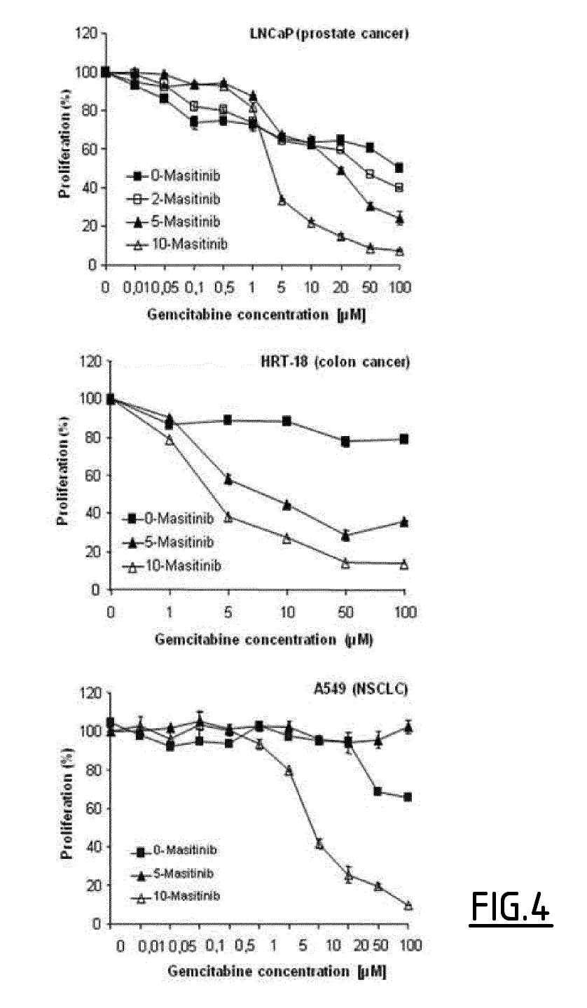

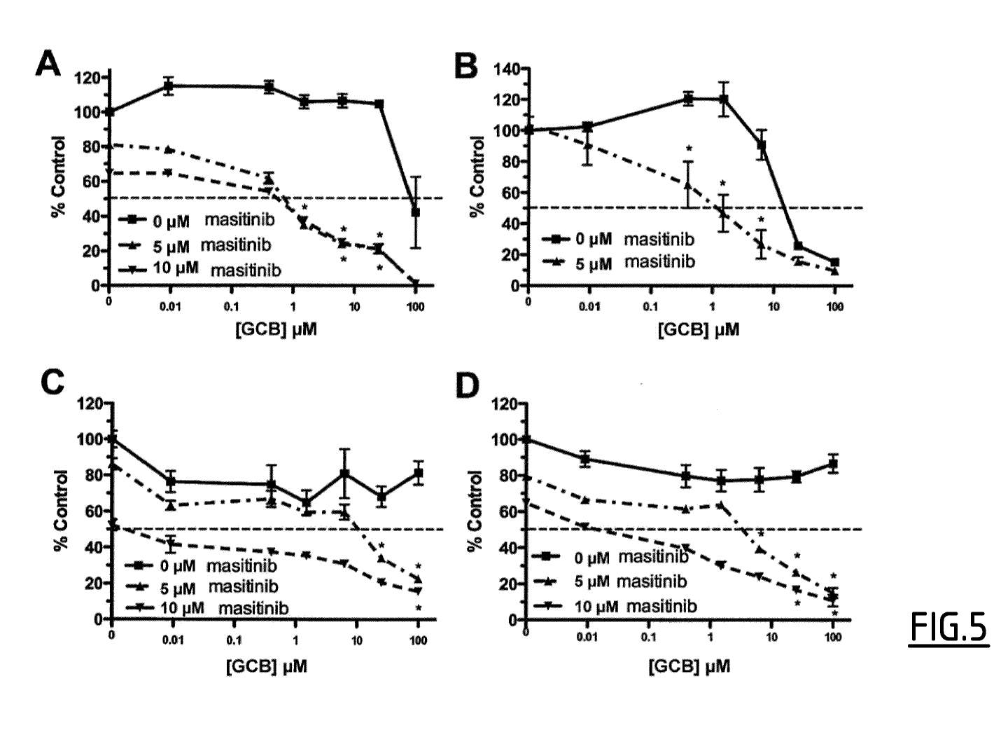

FIG. 4: Cell growth inhibition dose-response curves for gemcitabine. Masitinib enhances gemicitabine-induced growth inhibition.

FIG. 5: Cell growth inhibition dose-response curves for gemcitabine (GCB). Masitinib enhances gemicitabine-induced growth inhibition in canine osteosarcoma and breast carcinoma cell lines. (A) D17 osteosarcoma. (B) Abrams osteosarcoma. (C) CMT12 breast carcinoma. (D) CMT27 breast carcinoma. * Data points predicted to be synergistic based on Bliss analysis.

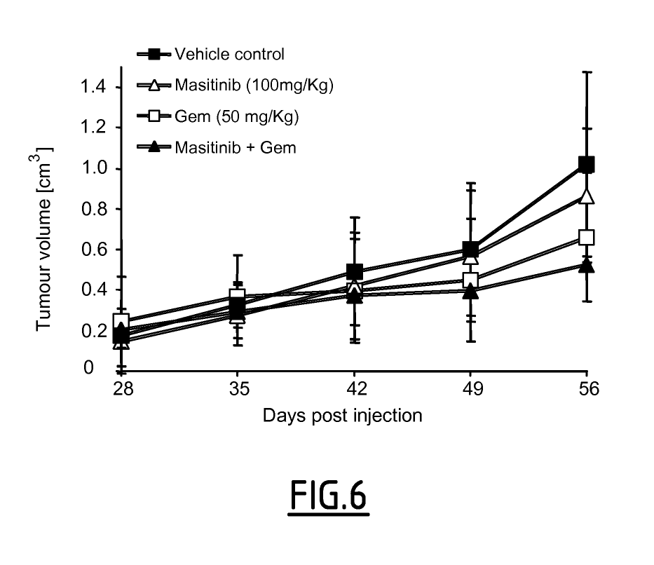

FIG. 6: In vivo anti-tumor activity of masitinib in a Nog-SCID mouse model of human pancreatic cancer.

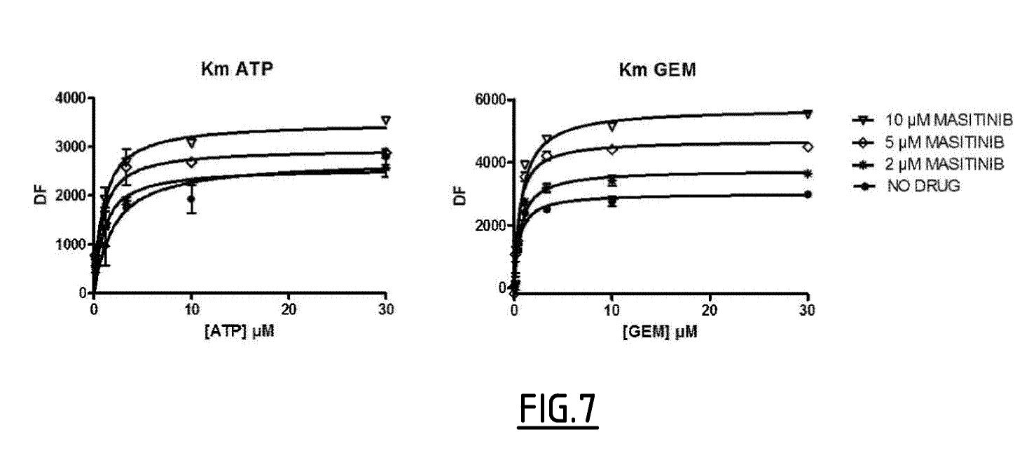

FIG. 7: Analysis of the effect of masitinib on dCK activity using ATP as the phosphate donor

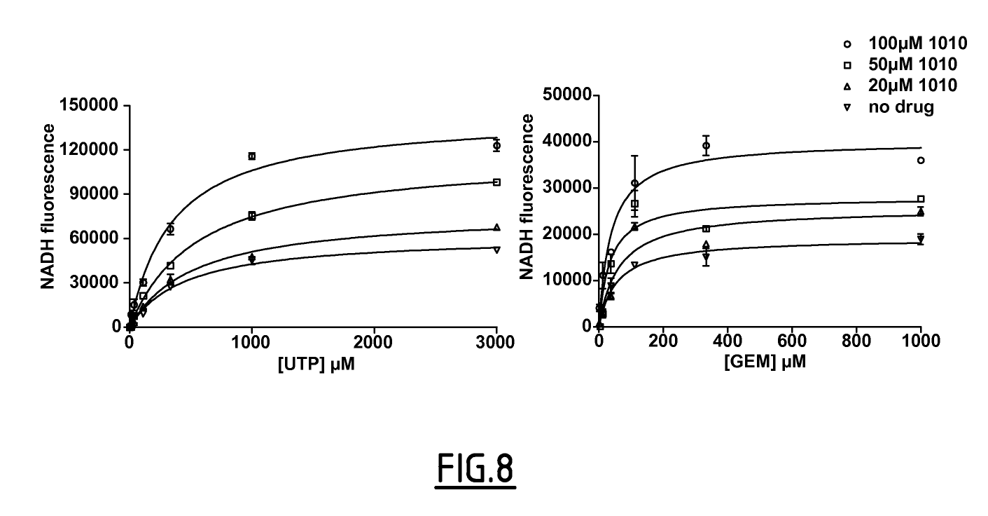

FIG. 8: dCK steady state kinetic in presence of UTP.

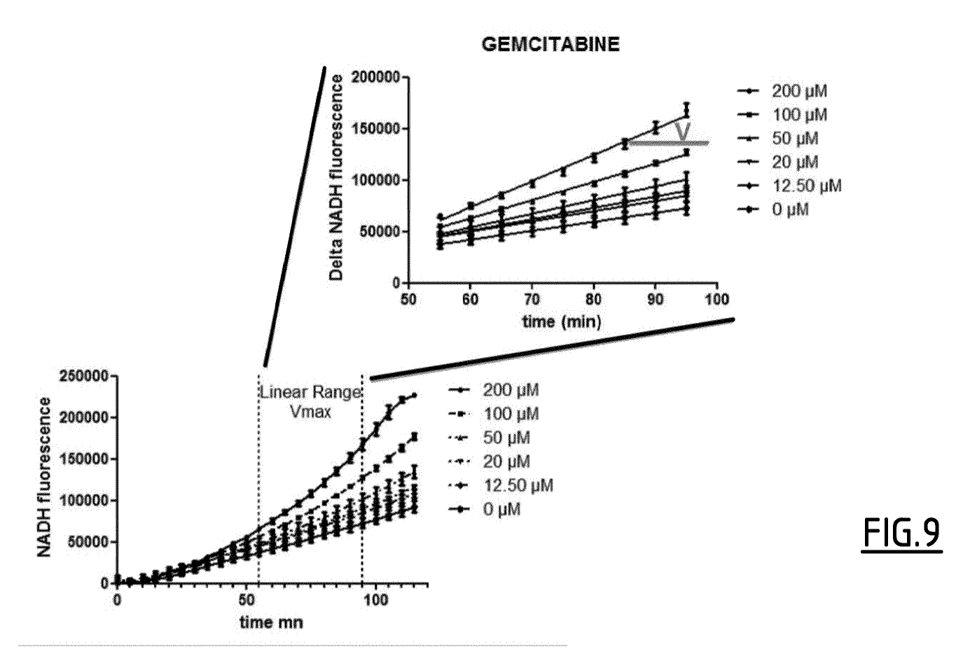

FIG. 9: Analysis of the effect of crescent dose of masitinib on the velocity of the phosphotransfer reaction catalyzed by dCK.

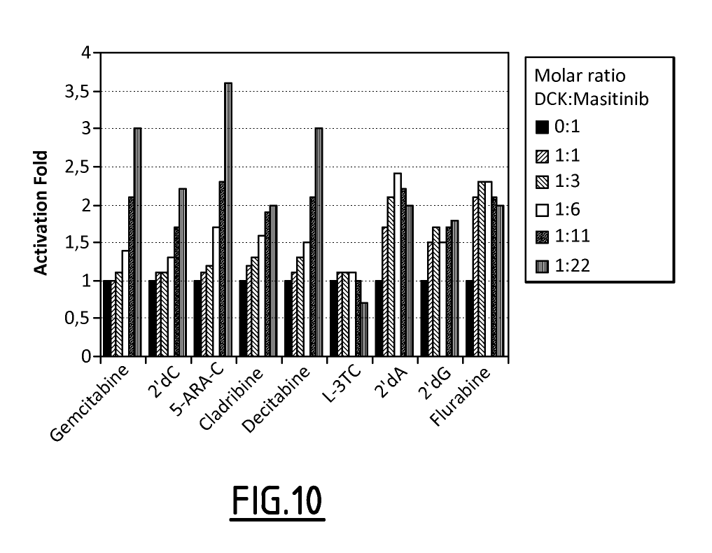

FIG. 10: Masitinib is global activator of dCK. Velocity was standardized with respect to the drug free control and the level of activation was defined as the ratio between the velocity at a given masitinib concentration and the velocity in the absence of drug. Concentration of the dCK substrate and dCK were held constant while varying the concentration of masitinib.

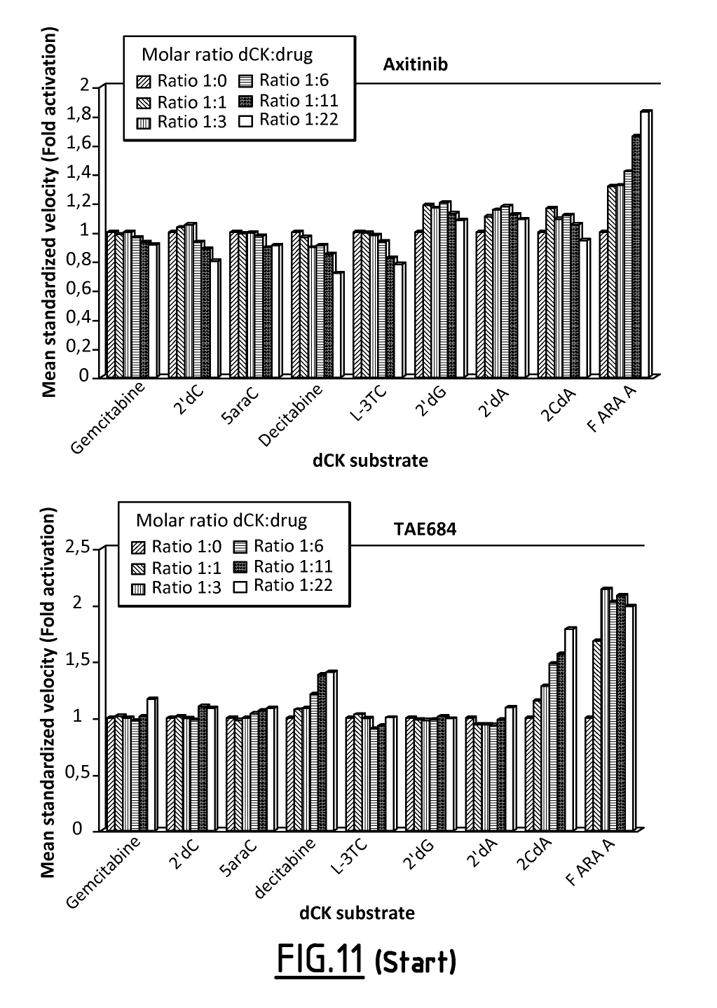

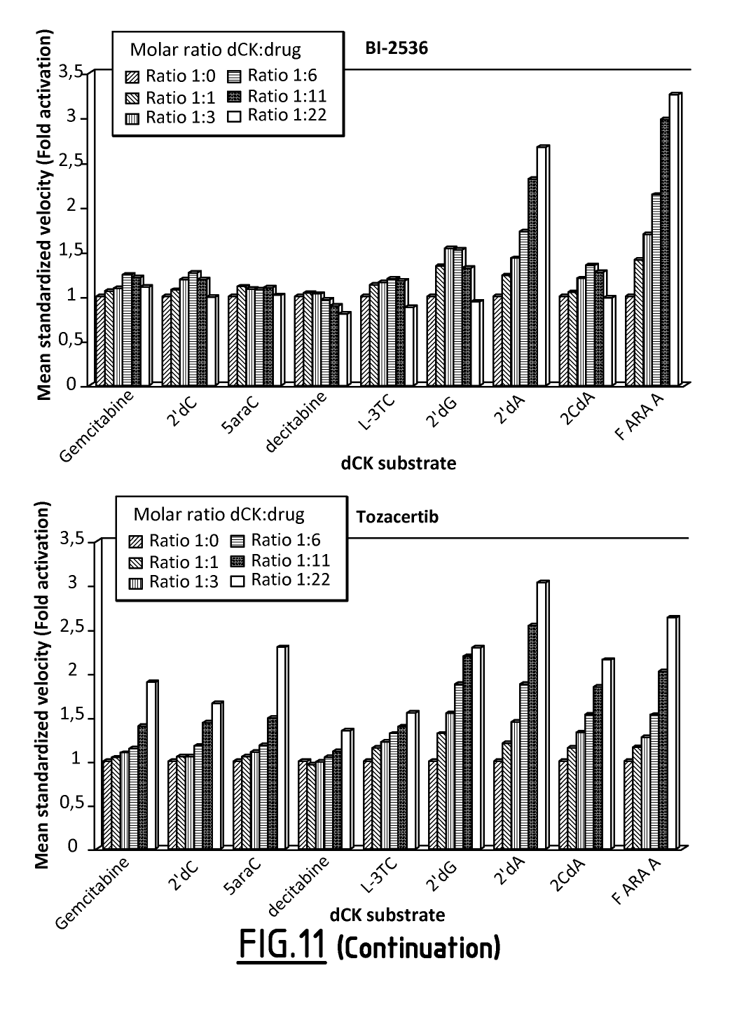

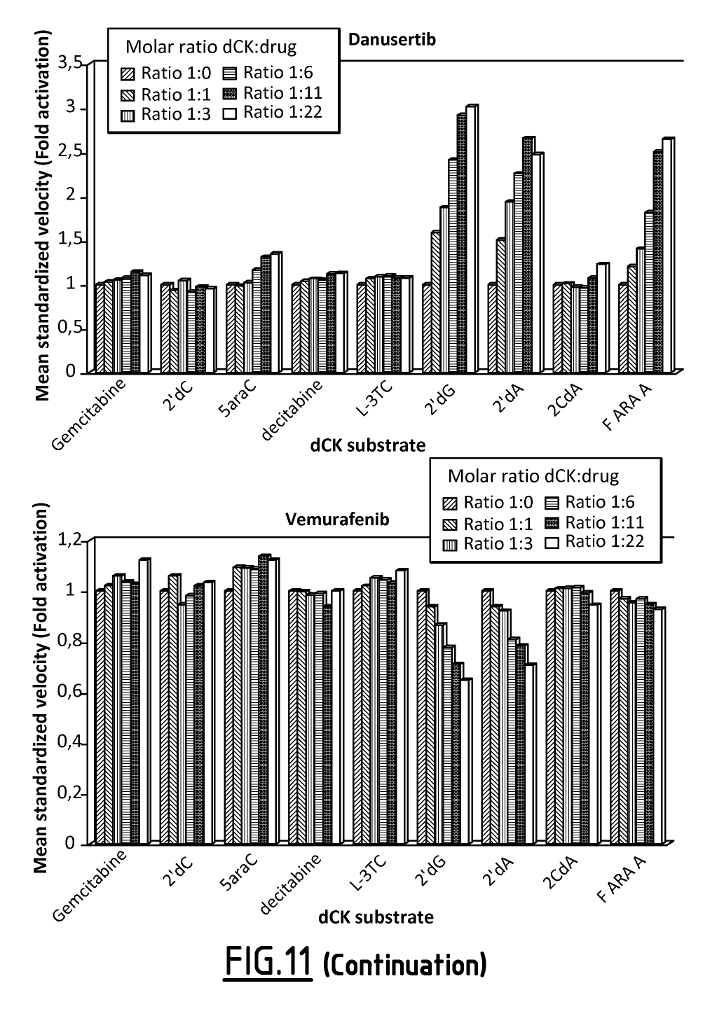

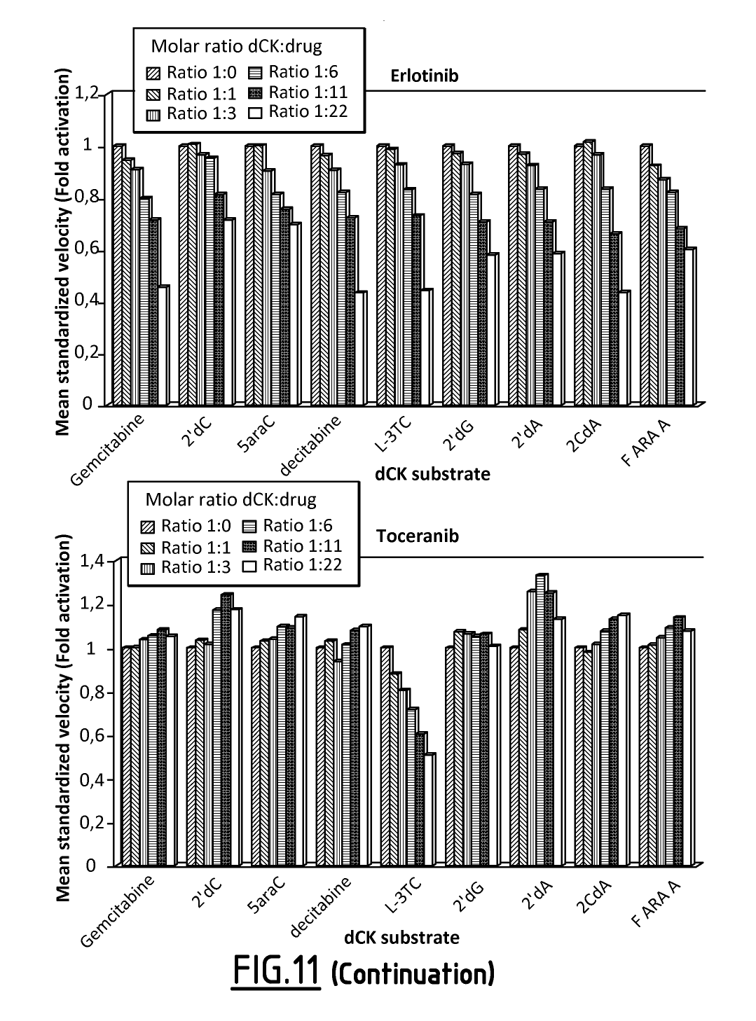

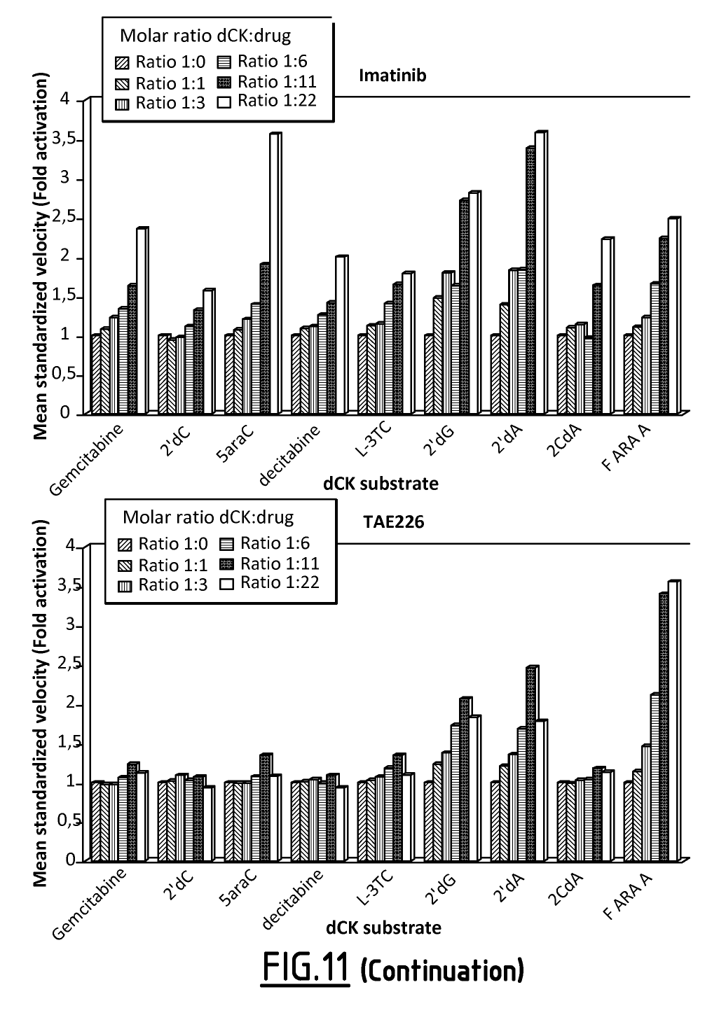

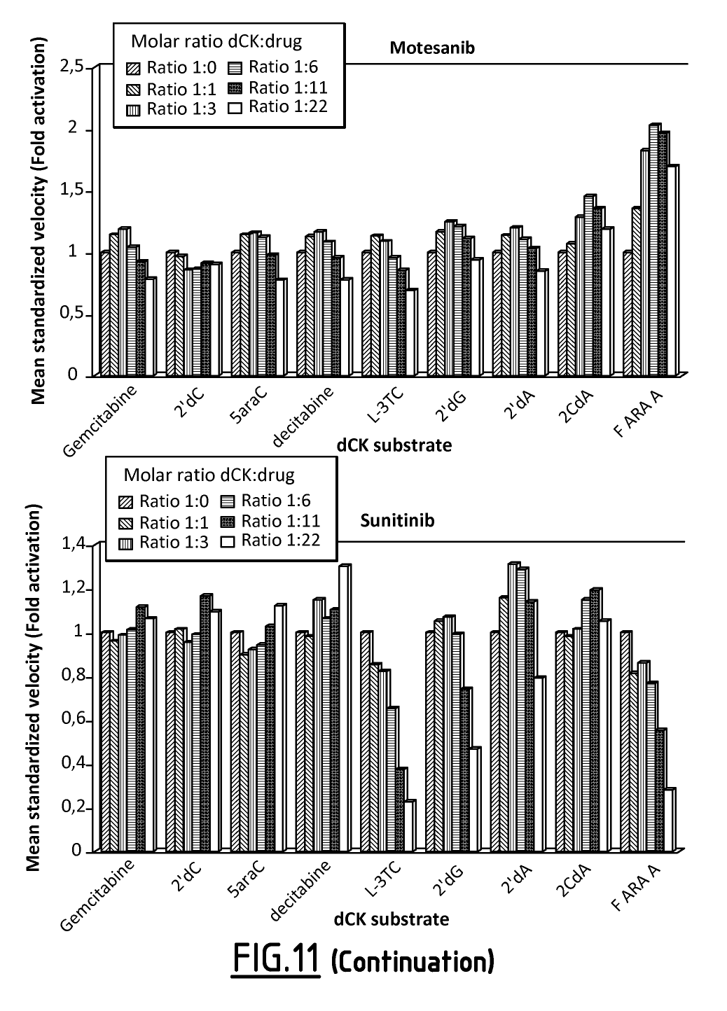

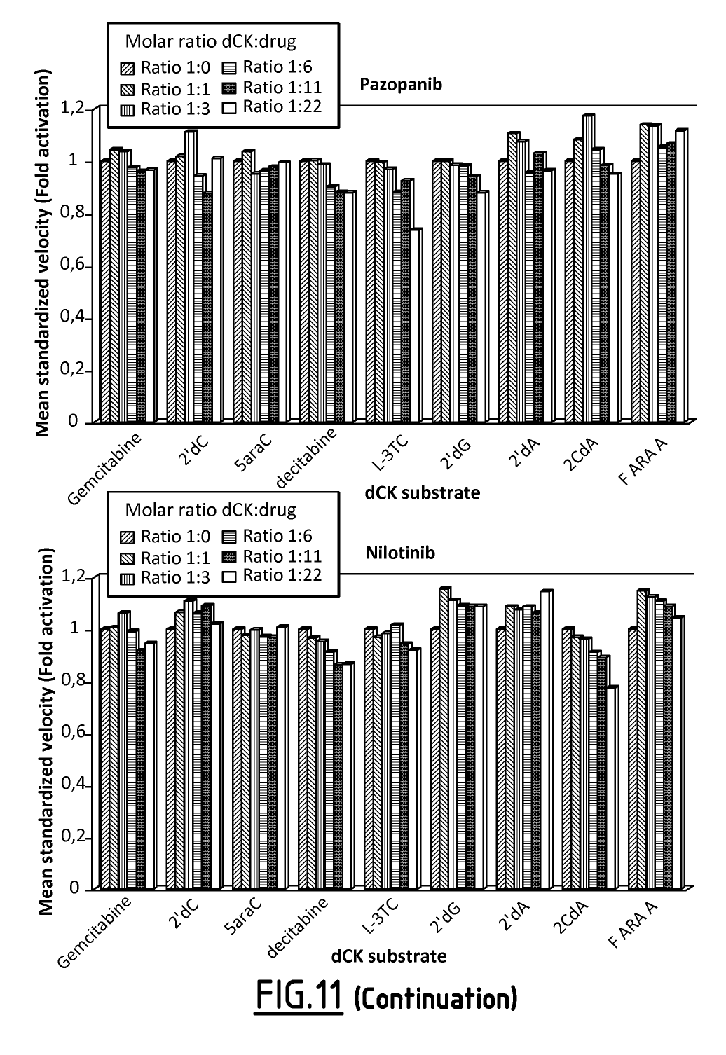

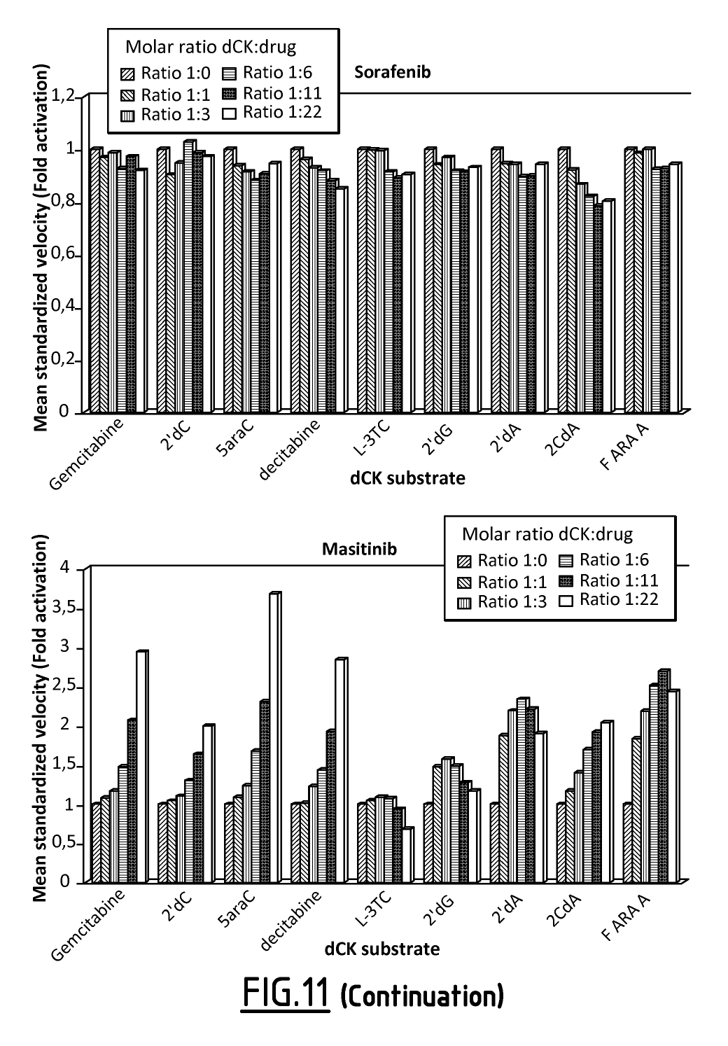

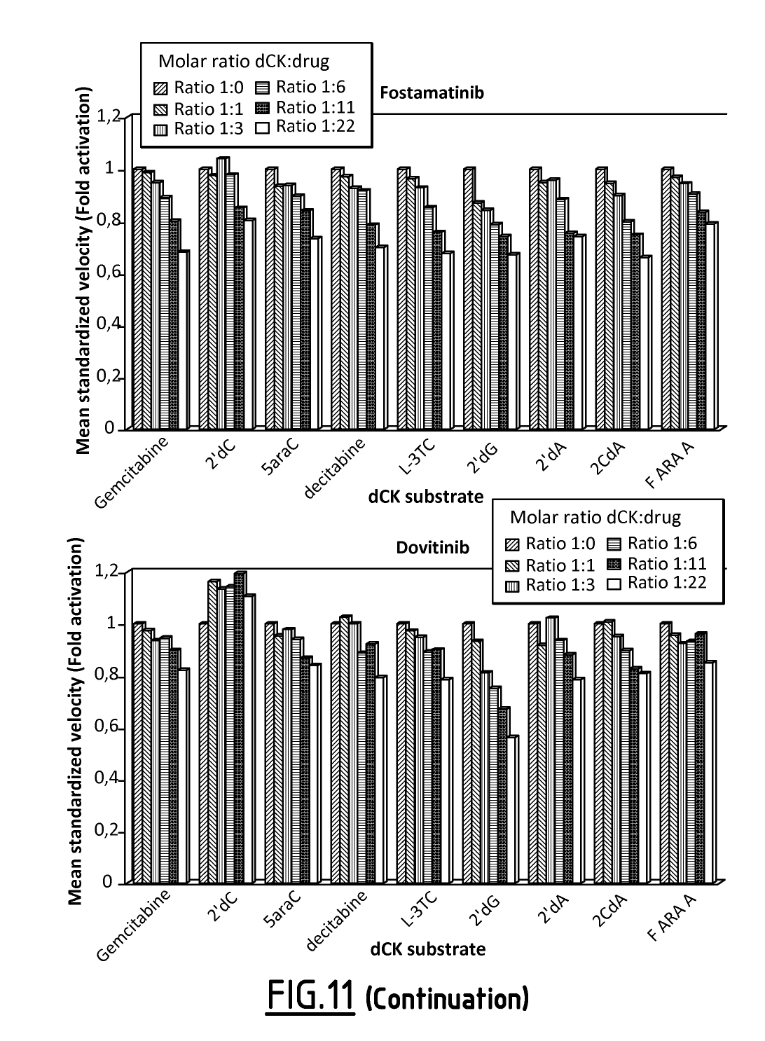

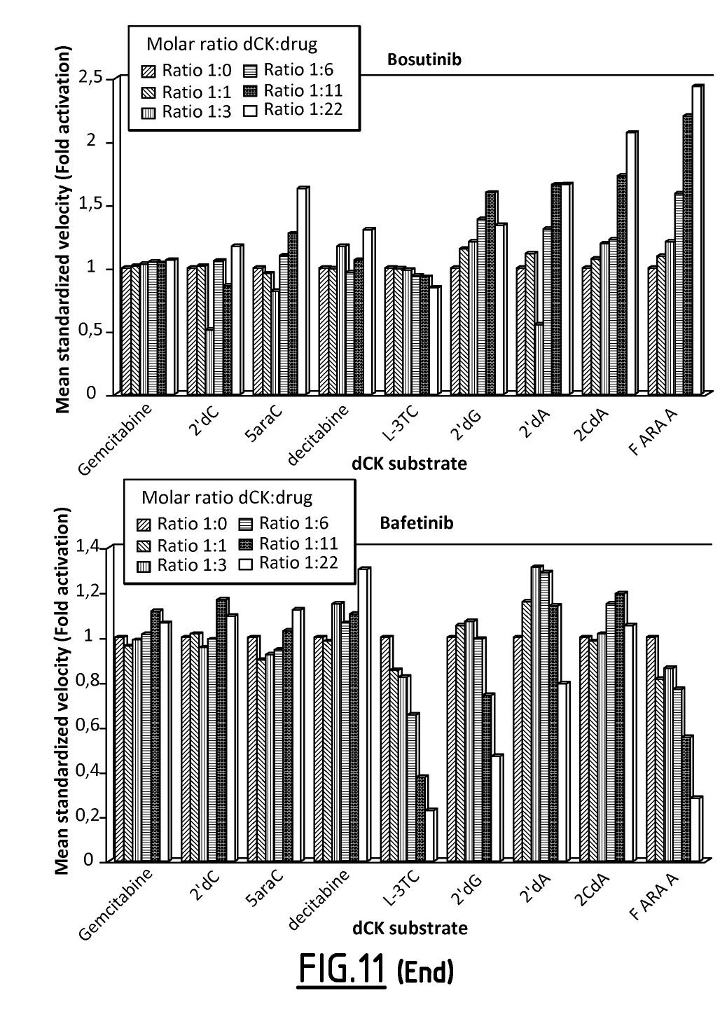

FIG. 11: Effect of various small molecule inhibitors/activators on different dCK substrates. Velocity was standardized with respect to the drug free control and the level of activation was defined as the ratio between the velocity at a given drug concentration and the velocity in the absence of drug. Concentration of the dCK substrate and dCK were held constant while varying the concentration of the small molecule inhibitor/activator under investigation.

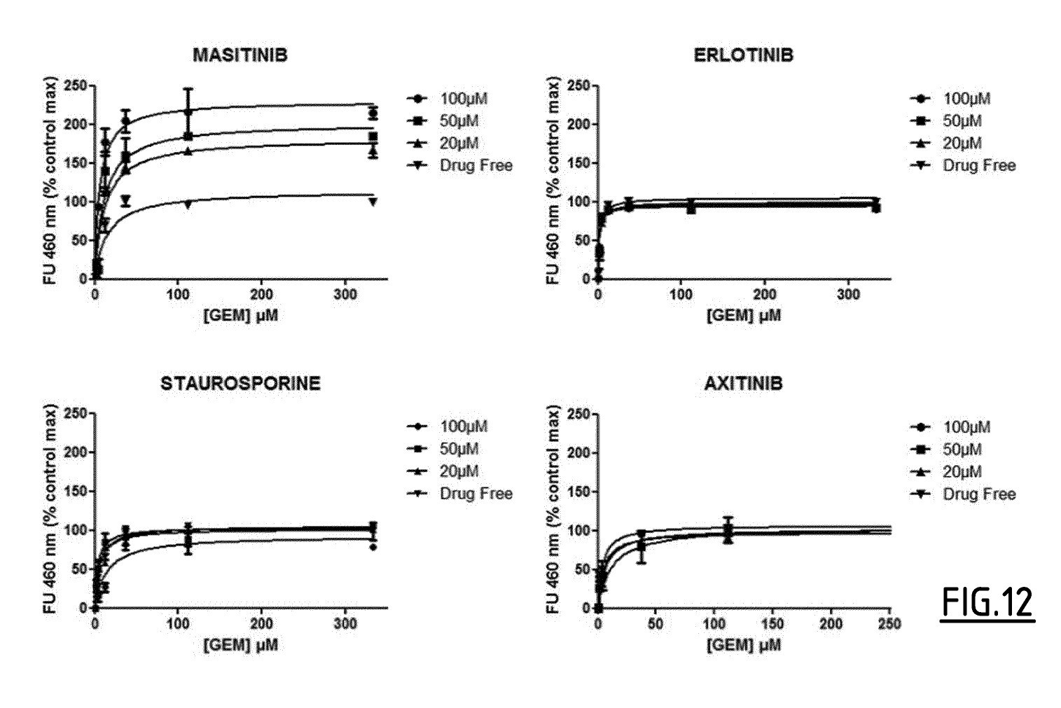

FIG. 12: Comparison of the effect of gemcitabine-enhancing cytotoxicity compounds on DCK activity. dCK (9 μM) was incubated in the presence of various amounts of gemcitabine and drug under investigation and 2 mM UTP.

The present invention is further illustrated by means of the following examples.

### Example 1

### In Vitro Study of Masitinib as a Chemosensitizer of Human Pancreatic Tumor Cell Lines

Preclinical studies were performed in vitro on human pancreatic tumor cell lines to evaluate the therapeutic potential of masitinib mesilate in pancreatic cancer, as a single agent and in combination with gemcitabine.

### Methods

Reagents: Masitinib (AB Science, Paris, France) was prepared from powder as a 10 or 20 mM stock solution in dimethyl sulfoxide and stored at −80° C. Gemcitabine (Gemzar, Lilly France) was obtained as a powder and dissolved in sterile 0.9% NaCl solution and stored as aliquots at −80° C. Fresh dilutions were prepared fcr each experiment.

Cancer cell lines: Pancreatic cancer cells lines (Mia Paca-2, Panc-1, BxPC-3 and Capan-2) were obtained from Dr. Juan Iovanna (Inserm, France). Cells were maintained in RPMI (BxPC-3, Capan-2) or DMEM (Mia Paca-2, Panc-1) medium containing glutamax-1 (Lonza), supplemented with 100 U/ml penicillin/100 μg/ml streptomycin, and 10% fetal calf serum (FCS) (AbCys, Lot S02823S1800). Expression of tyrosine kinases was determined by RT-PCR using Hot Star Taq (Qiagen GmbH, Hilden, Germany) in a 2720 Thermal Cycler (Applied Biosystems).

In vitro tyrosine phosphorylation assays: Mia Paca-2 cells (5×10⁶) were treated for 6 hours with increasing concentrations of masitinib in DMEM medium 0.5% serum. Cells were then placed on ice, washed in PBS, and lysed in 200 μl of ice-cold HNTG buffer (50 mM HEPES, pH 7, 50 mM NaF, 1 mM EGTA, 150 mM NaCl, 1% Triton X-100, 10% glycerol, and 1.5 mM MgCl2) in the presence of protease inhibitors (Roche Applied Science, France) and 100 μM Na3VO4. Proteins (20 μg) were resolved by SDS-PAGE 10%, followed by western blotting and immunostaining. The following primary antibodies were used: rabbit anti-phospho-GRB2 antibody (sc-255 1:1000, Santa Cruz, Calif.), and anti-phosphotyrosine antibody (4G10 1:1000, Cell Signaling Technology, Ozyme, France). These were followed by 1:10,000 horseradish peroxidase-conjugated anti-rabbit antibody (Jackson Laboratory, USA) or 1:20,000 horseradish peroxidase-conjugated anti-mouse antibody (Dako-France SAS, France). Immunoreactive bands were detected using enhanced chemiluminescent reagents (Pierce, USA).

Proliferation assays: Cytotoxicity of masitinib and gemcitabine was assessed using a WST-1 proliferation/survival assay (Roche diagnostic) in growth medium containing 1% FCS. Treatment was started with the addition of the respective drug. For combination treatment (masitinib plus gemcitabine), cells were resuspended in medium (1% FCS) containing 0, 5 or 10 μM masitinib and incubated overnight before gemcitabine addition. After 72 hours WST-1 reagent was added and incubated with the cells for 4 hours before absorbance measurement at 450 nm in an EL800 Universal Microplate Reader (Bio-Tek Instruments Inc.). Media alone was used as a blank and proliferation in the absence of compounds served as positive control. Results are representative of three/four experiments. The masitinib sensitization index is the ratio of the IC₅₀ of gemcitabine against the IC₅₀ of the drug combination.

### Results

Effect of masitinib on pancreatic cancer cells in vitro: PCR with gene-specific primers was performed to determine the expression profile of masitinib's targets in the human pancreatic cancer cell lines: Mia Paca-2, Panc-1, BxPC-3 and Capan-2. C-Kit was detectable in Panc-1 cells but was undetectable in all the other cell lines. PDGFRa was expressed in BxPC-3 and Panc-1 cells while PDGFRβ was mainly expressed in Panc-1 cells. A broader profile of tyrosine kinases revealed a strong expression of the EGFR family members ErbB1 and ErbB2, src family kinases Src and Lyn, FAK and FGFR3, in all four cell lines (FIG. 2A).

To estimate the range of masitinib concentration necessary to sensitize pancreatic tumor cell lines to chemotherapy, we assessed the ability of masitinib to block protein tyrosine phosphorylation by western blot analysis in cell lysates. FIG. 2B shows a strong pattern of protein tyrosine phosphorylation at baseline in Mia Paca-2 cells. Treatment with masitinib clearly inhibited tyrosine phosphorylation at 1 μM and beyond, demonstrating that masitinib is active at these concentrations. The control protein GRB2 remained unchanged under all treatment conditions. Similar results were obtained with the other pancreatic tumor cell lines. Based on these results, a masitinib concentration of up to 10 μM was considered appropriate to study its effect on cell proliferation.

The antiproliferative activity of masitinib or gemcitabine in monotherapy was assessed by WST-1 assays (FIGS. 3A and B). Masitinib did not significantly affect the growth of the tested cell lines, with an IC₅₀ of 5 to 10 μM. FIG. 3B shows that gemcitabine inhibits cell lines BxPC-3 and Capan-2 with an IC₅₀ of 2-20 μM, while Mia Paca-2 and Panc-1 cells show resistance (IC₅₀ >2.5 mM) as previously reported. Masitinib's potential to enhance gemcitabine cytotoxicity was assessed by pre-treating cell lines with masitinib overnight then exposing them to different doses of gemcitabine and recording the IC₅₀ concentrations. Table 5 summarizes the IC₅₀ of gemcitabine in the absence or presence of 5 and 10 μM masitinib. Mia Paca-2 cells, pre-treated with 5 and 10 μM masitinib, were significantly sensitized to gemcitabine, as evidenced by the substantial reductions (>400-fold reduction) in gemcitabine IC₅₀ (FIG. 4C). Panc1 cells were moderately sensitized (10-fold reduction) and no synergy was observed in the gemcitabine-sensitive cell lines Capan-2 and BxPC-3 (Table 5). These results suggest that pre-treatment with masitinib can restore cellular responsiveness to gemcitabine.

TABLE 5
IC₅₀ concentrations (μM) of various masitinib and/or gemcitabine
treatment regimens in different pancreatic cell lines.

| | | | Gemcitabine | Gemcitabine | Sensi- |
|---|---|---|---|---|---|
| | | | plus 5 μM | plus 10 μM | tization |
| | Masitinib | Gemcitabine | masitinib | masitinib | Index* |
| BxPC-3 | 5-10 | 10 | 10 | 10 | 1 |
| Capan-2 | 5-10 | 2 | 2 | NA | 1 |
| Mia | 5-10 | >10 | 1.5 | 0.025 | 400 |
| Paca-2 | | | | | |
| Panc-1 | 5-10 | >10 | 8 | 1 | 10 |

*Sensitization Index is defined as the IC₅₀ ratio of gemcitabine alone against the gemcitabine plus masitinib combination.
NA = Not available

Comparison of masitinib to other TKIs for their potential to sensitize gemcitabine-resistant pancreatic cancer cells: Similar TKI plus gemcitabine combination experiments to those described above were performed with gemcitabine-resistant Mia Paca-2 cells to compare masitinib with imatinib (Gleevec™, STI-571; Novartis, Basel, Switzerland), a TKI targeting ABL, PDGFR, and c-Kit); and dasatinib (Sprycel, Bristol-Myers Squibb), a TKI targeting SRC, ABL, PDGFR, and c-Kit. Mia Paca-2 cell proliferation was not inhibited by imatinib alone (10 μM), whereas it was partially inhibited in the presence of low concentrations of the SRC inhibitor dasatinib (>0.1 μM); albeit with <50% of the cells remaining resistant (FIG. 3D). This suggests that Mia Paca-2 cell growth is partly dependent on SRC, which is expressed at high levels in this cell line as shown in FIG. 2A. Pre-incubation of cells with 10 μM of imatinib or dasatinib did not result in an increased response of Mia Paca-2 cells to gemcitabine as compared to masitinib (FIG. 3D). Therefore, only masitinib was able to restore sensitivity to gemcitabine in Mia Paca-2 cells.

### Conclusion

The preclinical data reported here tentatively suggest that masitinib can reverse resistance to chemotherapy in pancreatic tumor cell lines. Further experimentation is however necessary to identify the mechanism of action responsible for this effect, to establish the wider proof-of-concept, and to determine how broadly applicable this combined treatment regimen may be, both in terms of possible drug combinations and disease indications.

### Example 2

### In Vitro Study of Masitinib as a Chemosensitizer of Human Tumor Cell Lines

Preclinical studies were performed in vitro on various human tumor cell lines to evaluate the therapeutic potential of masitinib mesilate in combination with gemcitabine for the treatment of breast cancer, prostate cancer, colorectal cancer, non-small cell lung cancer and ovarian cancer.

### Methods

Reagents: Masitinib (AB Science, Paris, France) was prepared from powder as a 10 or 20 mM stock solution in dimethyl sulfoxide and stored at −80° C. Gemcitabine (Gemzar, Lilly France) was obtained as a powder and dissolved in sterile 0.9% NaCl solution and stored as aliquots at −80° C. Fresh dilutions were prepared fcr each experiment. Cell lines: Colon and prostate cancer cell lines (Dr. Juan Iovanna, INSERM U624, Marseille, France), breast and ovarian cancer cell lines (Dr. Patrice Dubreuil, UMR 599 INSERM, Marseille, France), and lung cancer cell lines (Pr. Christian Auclair, UMR 8113 CNRS) were cultured as monolayers in RPMI 1640 medium containing L-glutamine supplemented with 100 U/ml penicillin and 100 μg/ml streptomycin, and 10% v/v heat-inactivated fetal calf serum (AbCys Lot S02823S1800) under standard culture conditions (5% CO2, 95% air in humidified chamber at 37° C.). In proliferation assays, all cells were grown in medium containing 1% FCS.

Cells survival and proliferation assays: Cytotoxicity of masitinib and chemotherapeutic agents were assessed using a WST-1 proliferation/survival assay (Roche diagnostic) in growth medium containing 1% FCS. Treatment was started with the addition of the respective drug. For combination treatment (masitinib plus chemotherapy), cells were resuspended in medium (1% FCS) containing 0, 5 or 10 μM masitinib and incubated over night before addition of cytotoxic agents. After 72 hours WST-1 reagent was added and incubated with the cells for 4 hours before absorbance measurement at 450 nm in an EL800 Universal Microplate Reader (Bio-Tek Instruments Inc.). Media alone was used as a blank and proliferation in the absence of compounds served as positive control (DMSO control). The new IC₅₀ was scored and the results are representative of 3-4 experiments. The masitinib sensitization index (SI) represents the ratio of the IC₅₀ of cytotoxic agent and the IC₅₀ of the drug combination.

### Results

When administered in combination with gemcitabine, masitinib sensitized human breast cancer cell lines, prostate cancer cell lines, colorectal cancer cell lines, non-small cell lung cancer cell lines, and ovarian cancer cell lines (Table 6). IC₅₀ is chemotherapy half inhibitory concentration for a fixed concentration of masitinib (5 or 10 μM). SI is the sensitization index (maximum sensitization reported) calculated as the IC₅₀ for the chemotherapeutic agent alone divided by the equivalent IC₅₀ in combination with masitinib.

Graphical representation of the gemcitabine data is shown in FIG. 4. Gemcitabine resistant cell lines LNCaP (prostate cancer) (A), HRT-18 (colon cancer) (B), and A549 (NSCLC) (C) were tested in proliferation assays in the presence and absence of masitinib at different concentrations. While gemcitabine could not induce apoptosis over a wide concentration ranges, addition of increasing doses of masitinib led to a shift of the respective IC₅₀ to lower gemcitabine concentrations.

### Conclusion

The preclinical data reported here tentatively suggest that masitinib can reverse resistance to chemotherapy and possibly generate synergistic growth inhibition in various human cancers, possibly through chemosensitization. Further experimentation is however necessary to identify the mechanism of action responsible for this effect, to establish the wider proof-of-concept, and to determine how broadly applicable this combined treatment regimen may be, both in terms of possible drug combinations and disease indications.

TABLE 6
Masitinib sensitization of various human cancer cell lines,
when administered in combination with gemcitabine
(maximum sensitization index shown).

| | | Gemcitabine | Sensitization |
|---|---|---|---|
| Cancer | Cell line | IC₅₀ (μM) | index |
| Breast cancer | MDAMB 231 | 100 | 2 |
| | MDAMB 134 | 100 | 2-10 |
| | BT20 | 50 | 5-10 |
| | BT474 | 100 | 2-10 |
| Prostate cancer | LnCaP | 25-100 | 5-20 |
| | DU145 | 100 | 5 |
| Ovarian cancer | OVCAR3 | 50 | 2.5 |
| Colorectal cancer | CaCo-2 | >100 | 2-5 |
| | HRT118 | >100 | 20 |
| NSCLC | A549 | 100 | 1-10 |
| | H1299 | 100 | 1-2 |
| | H1650 | 5 | 2.5 |

### Example 3

### In Vitro Study of Masitinib as a Chemosensitizer of Canine Tumor Cell Lines

The objective of this study was to evaluate masitinib's potential to sensitize various canine cancer cell lines to cytotoxic agents, including gemcitabine. Such chemosensitization, or synergistic growth inhibition, may allow lower concentrations of chemotherapeutic agent to be used, thereby reducing risk, or may increase the available efficacy at standard doses.

### Methods

We examined the ability of masitinib to inhibit the growth of a panel of canine cancer cells, including one canine mastocytoma cell line (C2), two osteosarcoma cell lines (Abrams and D17), two breast carcinoma cell lines (CMT12 and CMT27), a B-cell lymphoma line (1771), two hemangiosarcoma cell lines (DEN and FITZ), a histocytic sarcoma cell line (DH82), three melanoma cell lines (CML-6M, CML-10C2 and 17CM98), and two bladder carcinoma cell lines (Bliley and K9TCC).

A bioreductive fluorometric cell proliferation assay was used to assess the inhibitory activity of masitinib on cell proliferation and survival. To determine the half inhibitory concentration (IC₅₀) of masitinib as a single agent, cells were grown overnight in 96-well plates and then treated for 72 h with various concentrations of masitinib under standard conditions. For evaluation of masitinib's ability to synergize with various chemotherapeutic agents, each cell line was grown overnight in 96-well plates and then treated for 72 h with gemcitabine (0.01 to 100 μM), in the absence or presence of masitinib added at two concentrations near its IC₅₀ for each cell type. Relative viable cell number was assessed using Alamar Blue (Promega), expressed as a percentage of cells treated without chemotherapeutic agent.

The IC₅₀ was calculated for each cell line by nonlinear regression analysis fitting to a sigmoidal dose-response curve, using Prism v4.0b for Macintosh (GraphPad Software, Inc.). A sensitization factor was defined as the IC₅₀ for the chemotherapeutic agent alone divided by the equivalent IC₅₀ in combination with masitinib. The results are representative of at least three independent experiments. In order to determine whether the addition of masitinib to cytotoxic chemotherapy synergistically enhanced antiproliferative activity, the Bliss independence model was utilized. Differences between treatment groups (Bliss theoretical vs. experimental) were assessed using 2-way ANOVA and a Bonferroni post test.

### Results

The IC₅₀ for masitinib in C2 mastocytoma cells was 0.03 μM, whereas in all other cell lines tested, the IC₅₀ was between 5 and 20 μM (Table 7). The high sensitivity of the C2 cells to masitinib is expected because their proliferation is dependent on mutant c-Kit, masitinib's main kinase target. For this study, the activity of masitinib in C2 cells served as a positive control to compare the relative sensitivity of other canine tumor cell lines to masitinib monotherapy.

The maximum sensitization factor for each of those combinations showing synergistic activity is presented in Table 7. Sensitivity to gemcitabine was greatly enhanced by masitinib in four cell lines (FIG. 5); namely, the CMT27 and CMT12 breast carcinoma cell lines, and the D17 and Abrams osteosarcoma cell lines (sensitization factor of >75, >10, 70, and 18, respectively).

### Conclusions

The preclinical data reported here tentatively suggest that masitinib in combination with chemotherapeutic agents can generate synergistic growth inhibition in various canine cancers, possibly through chemosensitization. Masitinib appeared to sensitized osteosarcoma and mammary carcinoma cells to gemcitabine (>70-fold reduction at 5-10 μM). It is plausible that a masitinib/gemcitabine combination may be useful for treatment of osteosarcoma and mammary carcinoma. Further experimentation is however necessary to identify the mechanism of action responsible for this effect, to establish the wider proof-of-concept, and to determine how broadly applicable this combined treatment regimen may be, both in terms of possible drug combinations and disease indications.

TABLE 7
Chemosensitization of canine tumor cell lines by masitinib in combination with
gemcitabine, according to maximum sensitization factor.

| | | IC₅₀ masitinib | Masitinib | Combination | |
|---|---|---|---|---|---|
| Chemotherapeutic | | monotherapy | concentration in | IC₅₀ | Sensitization |
| agent | Cell line | (μM) | combination (μM) | (μM)^{a} | factor^{b} |
| Gemcitabine | Abrams | >10 | 5 | 1.0 | 18 |
| | D17 | >10 | 5 | 1.3 | 70 |
| | CMT12 | 8 | 10 | 10.8 | >10 |
| | CMT27 | 8 | 10 | 1.3 | >75 |
| | C2 | 0.03 | 0.001 | >100 | 1 |

^{a}Combination IC₅₀ refers to the variable concentration of chemotherapeutic agent in combination with a fixed concentration of masitinib.
^{b}The sensitization factor was calculated as the IC₅₀ for the chemotherapeutic agent alone divided by the equivalent IC₅₀ in combination with a fixed concentration of masitinib. The combination resulting in the maximum sensitization is reported along with the associated concentration of masitinib. All combinations presented showed synergistic antiproliferative activity as determined by Bliss analysis. Results are representative of at least three independent experiments

### Example 4

### Effect of Masitinib on Human Pancreatic Cancer In Vivo in a Nog-SCID Mouse Model

Preclinical studies were performed in vivo using a mouse model of human pancreatic cancer to evaluate the therapeutic potential of masitinib mesilate in pancreatic cancer, as a single agent and in combination with gemcitabine.

### Methods

Masitinib (AB Science, Paris, France) was prepared from powder as a 10 or 20 mM stock solution in dimethyl sulfoxide and stored at −80° C. Gemcitabine (Gemzar, Lilly France) was obtained as a powder and dissolved in sterile 0.9% NaCl solution and stored as aliquots at −80° C. Fresh dilutions were prepared for each experiment.

Pancreatic cancer cells lines (Mia Paca-2, Panc-1, BxPC-3 and Capan-2) were obtained from Dr. Juan Iovanna (Inserm, France). Cells were maintained in RPMI (BxPC-3, Capan-2) or DMEM (Mia Paca-2, Panc-1) medium containing glutamax-1 (Lonza), supplemented with 100 U/ml penicillin/100 μg/ml streptomycin, and 10% fetal calf serum (FCS) (AbCys, Lot S02823S1800). Expression of tyrosine kinases was determined by RT-PCR using Hot Star Taq (Qiagen GmbH, Hilden, Germany) in a 2720 Thermal Cycler (Applied Biosystems).

Male Nog-SCID mice (7 weeks old) were obtained from internal breeding and were housed under specific pathogen-free conditions at 20±1° C. in a 12-hour light/12-hour dark cycle and ad libitum access to food and filtered water. Mia Paca-2 cells were cultured as described above. At day 0 (D0), mice were injected with 107 Mia Paca-2 cells in 200 μl PBS into the right flank. Tumors were allowed to grow for 1.5 to 4 weeks until the desired tumor size was reached (˜200 mm³). At day 28, animals were allocated into four treatment groups (n=7 to 8 per group), ensuring that each group's mean body weight and tumor volume were well matched, and treatment was initiated for a duration of 4 to 5 weeks. Treatments consisted of either: a) daily sterile water for the control group, b) an intraperitoneal (i.p.) injection of 50 mg/kg gemcitabine twice a week, c) daily gavage with 100 mg/kg masitinib, or d) combined i.p injection of 50 mg/kg gemcitabine twice a week and daily gavage with 100 mg/kg masitinib. Tumor size was measured with calipers and tumor volume was estimated using the formula: volume=(length×width2)/2. The tumor growth inhibition ratio was calculated as (100)×(median tumor volume of treated group)/(median tumor volume of control group). Relative changes in tumor volumes were compared between treatment groups using a variance analysis (ANOVA). Normality of relative changes in tumor volumes between day 28 and day 56 was first tested using the Shapiro-Wilk test of normality. In case of a positive treatment effect, treatment groups were compared two-by-two using Tukey's multiple comparison test. A p-value <0.05 was considered as significant.

### Results

Preliminary experiments showed the optimal doses to use in this model (in terms of the combination's response and risk) were, masitinib at 100 mg/kg/day by gavage and gemcitabine at 50 mg/kg twice weekly by i.p. injection (data not shown). Tumors of the desired size (200 mm³) were obtained 28 days following Mia Paca-2 cell injection. The tumor size was monitored every 7 days until day 56, after which time the animals were sacrificed. FIG. 6 shows stabilization of tumor growth between day 35 and 49 in mice treated with gemcitabine or gemcitabine plus masitinib. Tumor response for each treatment group is reported in Table 8.

TABLE 8
Effect of masitinib plus gemcitabine on Mia Paca-2 pancreatic tumors
in Nog-SCID mice following 28 days of treatment.

| | | Tumor Volume | | Relative change in | |
|---|---|---|---|---|---|
| Treatment | Response | (mm³) | | volume (%) | |
| group | rate | Median | Range | Mean ± SD | Range |
| Control | 0/7 (0%) | 1023 | 711-1422 | 5.4 ± 2.3 | 2.8-9.0 |
| Masitinib | 3/7 (43%) | 865 | 450-1543 | 4.8 ± 1.4 | 2.6-6.6 |
| (100 mg/kg) | | | | | |
| Gemcitabine | 6/8 (75%) | 662* | 353-1317 | 2.1 ± 1.1 | 0.7-3.6 |
| (50 mg/kg) | | | | | |
| Masitinib + | 6/8 (75%) | 526* | 166-1190 | 2.4 ± 1.8 | 0.0-5.3 |
| Gemcitabine | | | | | |

*p-value <0.05 versus control using Tukey's multiple comparison test. Responders are defined as having a smaller tumor volume than the lower range limit of the control group (i.e. 711 mm³). Relative change in tumor volume measured from day 28 to day 56.

Mia Paca-2 tumor cells (10⁷) were injected into the flank of Nog-SCID mice. Treatment was initiated 28 days after tumor cell injection. The different groups were treated with either: twice weekly injections of gemcitabine (i.p. 50 mg/kg), daily oral masitinib (100 mg/kg), water (control), or combined daily oral masitinib (100 mg/kg) and twice weekly injections of gemcitabine. Mice were treated for 56 days.

The antitumor effect continued until day 56 (28 days of treatment) with better control of tumor growth evident in mice treated with the gemcitabine plus masitinib combination, as compared to the masitinib monotherapy or the control groups. Overall response analysis at day 56 defined a responder as having a smaller tumor volume than the lower range limit of the control group (i.e. 711 mm³). Following 28 days of treatment, 3/7 mice (43%) treated with masitinib alone were responders, with 6/8 mice (75%) responding in both the gemcitabine monotherapy and masitinib plus gemcitabine groups. Median tumor volumes were significantly reduced in the gemcitabine monotherapy and masitinib plus gemcitabine groups relative to control (p<0.05 Tukey's multiple comparison test). Although statistical significance was not demonstrated (p>0.05), the combination of masitinib plus gemcitabine appeared more potent than gemcitabine alone, with this observed trend being consistent over two separate experiments.

### Conclusion

The preclinical data reported here tentatively suggest that masitinib can reverse resistance to chemotherapy in pancreatic tumor cell lines. Further experimentation is however necessary to identify the mechanism of action responsible for this effect, to establish the wider proof-of-concept, and to determine how broadly applicable this combined treatment regimen may be, both in terms of possible drug combinations and disease indications.

### Example 5

### Studies Identifying the Mechanism of Action Responsible for the (Re)Sensitization Effect of Small Molecule Inhibitors/Activators in Combination with (Deoxy)Nucleotide or (Deoxy)Nucleoside Analog Drugs

Preliminary data (Examples 1 to 4) tentatively suggest that masitinib can reverse resistance to chemotherapy in various tumors. If these observations are confirmed via extensive clinical trials or discovery of a novel mechanistic data, the combination therapy of small molecule inhibitors/activators plus at least one anticancer or antiviral agent s would represent an innovative treatment option for a plurality of diseases. We hypothesized that masitinib specifically targets a protein that is responsible of this beneficial effect. To discover what this original mechanism of action is we have conducted studies designed to identify previously unknown targets (kinase or non kinase) responsible for this effect by a reverse proteomic approach. For the first time the deoxynucleoside kinase dCK has been positively identified as one of the masitinib-interacting proteins (secondary target). We have therefore characterized the effect of masitinib on the nucleoside and nucleoside like prodrugs-phosphorylation activity of human deoxycytidine kinase. Findings have clearly demonstrated that masitinib enhances the dCK-dependent activation of the pro-drug gemcitabine independently of the phosphate donor (ATP or UTP). Moreover, masitinib also activates the dCK dependent phosphorylation of various substrates including the physiological substrates (2′deoxycytidine, 2′deoxyguanosine and 2′deoxyguanosine) and several prodrugs of therapeutic interest such as cladribine and cytosine arabinoside. From these results it should be consider that masitinib is an activator of hdCK and therefore a potentiator of (deoxy)nucleotide or (deoxy)nucleoside analog agents.

### Methods

A technique based upon reverse proteomic technology has previously been shown capable of identifying subtle differences in protein-drug interaction profile between inhibitors/activators with very close selectivity profiles [Rix et al. Blood 2007. 110:4055-4063]. We have adapted this technique with the objective of identifying possible mechanisms of actions that might confirm our hypothesis of an enhanced or synergistic effect between small molecule inhibitors/activators and anticancer or antiviral agents, such as (deoxy)nucleotide or (deoxy)nucleoside analog drugs.

### dCK Cloning, Expression and Purification

hDCK cDNA was Gateway® cloned into the pDEST 17 vector (Invitrogen) from the IMAGE cDNA clone BC103764, leading to the expression of a NH2-hexahistidine-tagged full length enzyme. The protein was expressed in the BL21 AI (Arabinose induced) E. coli strain (Invitrogen) before a one-step purification by nickel affinity chromatography on a Histrap crude 1 ml column (GE healthcare life sciences). dCK was purified to homogeneity.

Substrate Characteristics with dCK Using ATP as the Phosphate Donor.

The analysis of the effect of masitinib on dCK activity using ATP as phosphate donor was assayed with the HTRF® Transcreener® ADP assay (Cisbio International). It is an immunoassay based on the competition between the native ADP (generated by the reaction of transfer of phosphate catalyzed by dCK) and a fluorescent tracer the ADP-d2. ADP and ADP-d2 compete for the binding to a monoclonal anti-ADP antibody labeled with Europium (Eu3+) cryptate. This assay comprises two steps: (1) an enzymatic step during which the substrate is incubated with dCK in the presence of ATP and Mg2+, leading to the generation of native ADP; (2) at the end of the reaction (stopped by addition of EDTA, which chelates Mg2+) the antibody anti-ADP-Eu3+(emission wavelength 620 nm) is added in the presence of the fluorescent tracer ADP-d2 (emission wavelength 665 nm). The obtained signal is inversely proportional to the concentration of ADP in the sample. All measurements were performed on a BMG Labtech Pherastar FS apparatus. Results are expressed in delta fluorescence (DF) unit defined as follow DF %=[(ratio-ratio blank)/(ratio blank)]*100, where ratio=(665 nm/620 nm)*10⁴.

Substrate Characteristics with dCK Using UTP as the Phosphate Donor.

Analysis of the effect of masitinib on dCK activity using UTP as phosphate donor was performed using a spectrophotometric continuous enzymatic-coupled assay based on the conversion of phosphoenolpyruvate (PEP) and UDP to pyruvate and UTP by pyruvate kinase (PK) and the subsequent conversion of pyruvate to lactate by lactate dehydrogenase (LDH). The latter step requires NADH+, which is oxidized to NAD+. NADH is a fluorescent molecule with a 337 nm excitation wavelength and a maximum emission peak at 460 nm. By contrast, NAD+, the oxidized form of the coenzyme, does not fluoresce. Thus, the measurement of decrease in the fluorescent emission (wavelength 460 nm) can be converted into kinase activity where one molecule of NADH oxidized to NAD+ corresponds to the production of one molecule of UDP by dCK. All experiments were performed in 50 mM HEPES, 5 mM MgCl2, 1 mM DTT, 0.01% BRIJ-35 buffer supplemented by DCK at 9 μM, dCK substrate and masitinib at varying concentrations. All measurements were performed on a BMG Labtech Pherastar FS apparatus. All assays were performed in triplicate or quadruplicate and each experiment was performed at least twice. Km and Vmax values were determined using PRISM software (GraphPad Software Inc, La Jolla, Calif.) by fitting the experimental data according to Michaelis-Mentem approximation defined as v=Vmax*[S]/Km+[S].

### Analysis of the Effect of Masitinib on the Phosphorylation of Pemcitabine by dCK in Presence of ATP

Preliminary experiments to determine dCK steady state kinetic parameters in the presence of ATP showed that the experimental conditions of 100 μM ATP, 1 mM gemcitabine, and 10 nM dCK, corresponded to a steady state kinetic. That is to say, a 10 nM dCK working concentration ensures a linear reaction rate and a good assay window. The Km values with respect of gemcitabine (Km=1±0.3 μM) and ATP (Km=1.5±0.2 μM) were consistent with previously published values. The effect of various concentration of masitinib on dCK activity was analyzed by co-varying either gemcitabine or ATP in presence of a fixed concentration of masitinib (2, 5, or 10 μM). The results presented in FIG. 7 show that crescent concentrations of masitinib lead to an augmented maximum velocity of the reaction (Vmax). This result clearly indicates that masitinib directly enhance dCK enzymatic activity.

The Vmax and Km values summarized in Table 9, illustrate that the binding of masitinib to dCK results in a strong augmentation of reaction velocity (2-fold) without significantly affecting the Km values with respect to ATP and gemcitabine. This indicates that masitinib activates dCK by acting on the enzyme turnover (Kcat=Vmax/[E]).

TABLE 9

| | Effect of masitinib on velocity and Km with respect of ATP and gemcitabine | | | | | | | | | | | | | | | |
|---|---|---|---|---|---|---|---|---|---|---|---|---|---|---|---|---|
| | ATP | | | | | | | | Gemcitabine | | | | | | | |
| | 10 μM | | 5 μM | | 2 μM | | 0 μM | | 10 μM | | 5 μM | | 2 μM | | 0 μM | |
| | Masitinib | | Masitinib | | Masitinib | | Masitinib | | Masitinib | | Masitinib | | Masitinib | | Masitinib | |
| Vmax | 3490 ± | 107 | 2953 ± | 170 | 2554 ± | 160 | 2715 ± | 250 | 5720 ± | 190 | 4702 ± | 141.7 | 3754 ± | 133 | 3005 ± | 117 |
| Km | 0.9 ± | 0.14 | 0.8 ± | 0.24 | 1 ± | 0.3 | 2 ± | 0.76 | 0.77 ± | 0.12 | 0.52 ± | 0.075 | 0.64 ± | 0.1 | 0.52 ± | 0.1 |

| | | | | | | | | |
|---|---|---|---|---|---|---|---|---|
| R² | 0.9745 | 0.9077 | 0.9071 | 0.8545 | 0.9666 | 0.9655 | 0.9587 | 0.9462 |

### Analysis of the Effect of Masitinib on the Phosphorylation of Gemcitabine by dCK in Presence of UTP

It has been described previously that UTP is the preferred phosphoryl donor for dCK, thus, analysis of the effect of masitinib on the phosphorylation of dCK substrates in the presence of UTP was performed. Preliminary experiments to determine optimal dCK assay conditions in the presence of UTP showed that the experimental conditions of 2 mM UTP, 1 mM dCK substrate, and 9 μM dCK corresponded to a steady state kinetic. The effect of masitinib on dCK activity was analyzed by co-varying either the dCK substrate or UTP in presence of a fixed concentration of masitinib (20, 50 or 100 μM). In general, all UTP experiments were performed by incubating 9 μM of dCK with 1 mM of a dCK substrate under investigation (e.g. gemcitabine), 2 mM UTP, and various amounts of masitinib for 2 hours at room temperature. The velocity of subsequent reactions was calculated as the slope of the linear range of each kinetic curve (according to v=d[P]/dt). FIG. 8 shows that crescent concentrations of masitinib lead to a 2-fold augmentation of gemcitabine's reaction's maximum velocity, without significantly affecting the Km values with respect of both UTP and gemcitabine. The Vmax and Km values summarized in Table 10, illustrate that masitinib enhances the dCK enzymatic activity in the presence of UTP.

TABLE 10

| | Effect of masitinib on velocity and Km with respect of UTP and gemcitabine | | | | | | | | | | | | | | | |
|---|---|---|---|---|---|---|---|---|---|---|---|---|---|---|---|---|
| | UTP | | | | | | | | GEMCITABINE | | | | | | | |
| | 100 μM | | 50μM | | 20μM | | 0μM | | 100 μM | | 50μM | | 20μM | | 0μM | |
| | Masitinib | | Masitinib | | Masitinib | | Masitinib | | Masitinib | | Masitinib | | Masitinib | | Masitinib | |
| Vmax | 143293 ± | 5058 | 116756 ± | 2338 | 77994 ± | 3161 | 61941 ± | 2483 | 40156 ± | 2015 | 28014 ± | 1894 | 25572 ± | 2068 | 19136 ± | 1210 |
| Km | 350.8 ± | 41.17 | 562.6 ± | 33.26 | 529.8 ± | 64.69 | 451.2 ± | 57.14 | 38.16 ± | 9.272 | 34.71 ± | 11.72 | 61.92 ± | 19.66 | 55.48 ± | 16.20 |

| | | | | | | | | |
|---|---|---|---|---|---|---|---|---|
| R² | 0.9824 | 0.9960 | 0.9836 | 0.9832 | 0.9482 | 0.9194 | 0.8813 | 0.9428 |

The effect of masitinib was assayed on nine dCK substrates including the physiological substrates of 2′dC, 2′dA and 2′dG, and several prodrugs of therapeutic interest (gemcitabine, cladribine, fludarabine, lamivudine, cytosine arabinoside, and decitabine). Experimental results are exemplified by gemcitabine in FIG. 9.

For each dCK substrate and each concentration of masitinib, the velocity of the reaction was standardized with respect to the drug free control and velocity ratios were compared. FIG. 10 clearly shows that masitinib activates the phosphotransfer activity of dCK in a dose dependent manner, as evidenced by a 2-fold increase in the reaction's velocity with masitinib concentration. Activation is more pronounced (3-4 fold increase) for deoxycytidine-like substrates, such as gemcitabine and 5-ARA-C. One exception to the general observation of increased phosphotransfer activity was seen with lamivudine (L-3TC), although this can be explain by the fact that L-3TC is an L-nucleoside analog and therefore binds dCK differently from D-nucleoside analogs. These results show that masitinib is global activator of dCK.

Compounds with a structurally different scaffold from masitinib (including: axitinib, bafetinib, BI-2536, bosutinib, danusertib, dovitinib, erlotinib, fostamatinib, imatinib, motesanib, nilotinib, pazopanib, sorafenib, sunitinib, TAE226, TAE684, toceranib, tozacertib, vemurafenib) were investigated to evaluate their effect on substrate phosphorylation in the presence of UTP. FIG. 11 shows a summary of the effect of these different small molecule inhibitors/activators tested on nine different dCK substrates.

### Masitinib Sensitizes Cancer Cells to Gemcitabine by a Unique Mechanism

Several studies have reported that certain kinase inhibitors enhance gemcitabine cytotoxicity including the investigational drug staurosporine, axitinib and erlotinib. To date, erlotinib is the only kinase inhibitor approved for the treatment of pancreatic cancer in association with gemcitabine, however, its mechanism of action remains unclear. We have therefore investigated the effect of these three compounds and masitinib on the dCK enzymatic activation of gemcitabine (see FIG. 12). It is clear that these compounds, unlike masitinib, have no effect on dCK enzymatic activity since they do not affect either Km or Vmax values. Conversely, masitinib produced at least a 2-fold increase in Vmax. Our results confirm unambiguously that, among kinase inhibitors, masitinib has the unique property to directly activate gemcitabine dCK in vitro.

### Conclusion

We have positively identified that the deoxynucleoside kinase dCK is one of the masitinib-interacting proteins, with masitinib effectively up-regulating its activity. Thus, it appears that masitinib is capable of modulating dCK activity with a consequence that it can modulate phosphorylation of (deoxy)nucleotide or (deoxy)nucleoside analog drugs. These data also clearly establish that some structurally divergent kinase inhibitors are also capable of modulating dCK activities in the same manner as discovered for masitinib, albeit for a more limited range of dCK substrates. The most active compounds are masitinib, imatinib, BI-2536, bosutinib, danusertib, and tozacertib. However, such an effect is not a class/agent effect because the majority of kinase inhibitors/activators tested have relatively little or no activity, including dovitinib, erlotinib, fostamatinib, nilotinib, pazopanib, sorafenib, sunitinib, toceranib, and vemurafenib. This property of dCK regulation may be of great therapeutic benefit, either amplifying the effectiveness of dCK-associated therapeutic agents, such as but not limited to (deoxy)nucleotide or (deoxy)nucleoside analog drugs for the treatment of cancer (including hematological malignancies) or viral infections, reducing the risk of such therapeutic agents by maintaining effectiveness at lower doses, or by counteracting the effects of drug resistance.

1-146. (canceled)

147. A method for modulating deoxycitidine kinase activity in a human patient with cancer, thereby treating cancer, wherein said method comprises administering to the human patient at least one small molecule inhibitor/activator or a pharmaceutically acceptable salt or solvate thereof in combination with at least one anticancer drug, wherein said at least one small molecule inhibitor/activator is selected from imatinib, BI-2536, bosutinib, danusertib, tozacertib, and compounds of formula A: 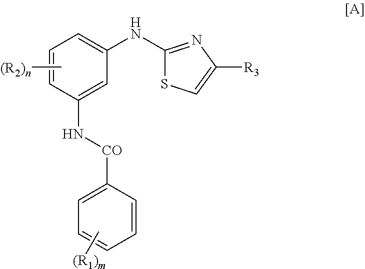
wherein:
R1 and R2 are selected independently from hydrogen, halogen, a linear or branched alkyl, cycloalkyl group containing from 1 to 10 carbon atoms, trifluoromethyl, alkoxy, cyano, amino, alkylamino, dialkylamino, solubilizing group,
m is 0-5 and n is 0-4,
R3 is one of the following:
(i) an aryl group such as phenyl or a substituted variant thereof bearing any combination, at any one ring position, of one or more substituents such as halogen, alkyl groups containing from 1 to 10 carbon atoms, trifluoromethyl, cyano and alkoxy;
(ii) a heteroaryl group such as 2, 3, or 4-pyridyl group, which may additionally bear any combination of one or more substituents such as halogen, alkyl groups containing from 1 to 10 carbon atoms, trifluoromethyl and alkoxy;
(iii) a five-membered ring aromatic heterocyclic group, which may additionally bear any combination of one or more substituents such as halogen, an alkyl group containing from 1 to 10 carbon atoms, trifluoromethyl, and alkoxy, or a pharmaceutically acceptable salt or solvent thereof.

148. The method according to claim 147, wherein said at least one small molecule inhibitor/activator or a pharmaceutically acceptable salt or solvate thereof is selected from the group consisting of masitinib, imatinib, BI-2536, bosutinib, danusertib, and tozacertib, pharmaceutically acceptable salts or solvates thereof.

149. The method according to claim 147, wherein said at least one anticancer drug is a (deoxy)nucleotide or (deoxy)nucleoside analog agent.

150. The method according to claim 147, wherein said at least one anticancer drug is a (deoxy)nucleotide or (deoxy)nucleoside analog drug selected from: gemcitabine, abacavir, acyclovir, adefovir, amdoxovir, apricitabine, azacitidine, Atripla®, capecitabine, cladribine, movectro, clevudine, clofarabine, evoltra, Combivir®, cytarabine, decitabine, didanosine, elvucitabine, emtricitabine, entecavir, Epzicom®, festinavir, fludarabine, fluorouracil, idoxuridine, KP-1461, lamivudine, nelarabine, racivir, ribavirin, sapacitabine, stavudine, taribavirin, telbivudine, tenofovir, tezacitabine, trifluridine, Trizivir®, troxacitabine, Truvada®, vidarabine, zalcitabine, or zidovudine.

151. The method according to claim 147, wherein said at least one anticancer drug is a (deoxy)nucleotide or (deoxy)nucleoside analog drug is selected from gemcitabine, azacitidine, capecitabine, clofarabine, cytarabine, decitabine, fludarabine, fluorouracile, nelarabine, sapacitabine, tezacitabine or troxacitabine.

152. The method according to claim 147, wherein said at least one anticancer drug is gemcitabine.

153. The method according to claim 147, comprising administering masitinib mesilate.

154. The method according to claim 147, wherein the daily or weekly dosage of said at least one anticancer drug is reduced by 50 to 95% of the manufacture's recommended dose with equivalent therapeutic effect.

155. The method according to claim 147, wherein the at least one small molecule inhibitor/activator or pharmaceutically acceptable salt or solvate thereof is administered at a dose of 6 to 12 mg/kg bodyweight/day.

156. The method according to claim 147, wherein the at least one small molecule inhibitor/activator or pharmaceutically acceptable salt or solvate thereof is administered at a starting dose of 6.0 mg/kg/day±1.5 mg/kg/day.

157. The method according to claim 147, wherein the at least one small molecule inhibitor/activator or pharmaceutically acceptable salt or solvate thereof is administered orally.

158. The method according to claim 147, wherein the at least one small molecule inhibitor/activator or pharmaceutically acceptable salt or solvate thereof is administered twice a day.

159. The method according to claim 147, said method comprising a long-term administration of said combination over more than 3 months.

160. The method according to claim 147, wherein the patient is a patient with an under-expression, down-regulation, or decreased activity of dCK.

161. The method according to claim 147, wherein said patient is either resistant or refractory or intolerant to said at least one anticancer drug.

162. The method according to claim 147, wherein said patient is either naïve to said at least one anticancer drug or is responding to treatment with said at least one anticancer drug.

163. The method according to claim 147, wherein said patient is in need of treatment for cancer (including hematological malignancies) selected from: acute lymphocytic leukemia (ALL), acute myelogenous leukemia (AML), adrenocortical carcinoma, anal cancer, B cell lymphoma, basal cell carcinoma, bile duct cancer, bladder cancer, bone cancer, brainstem glioma, brain tumor, breast cancer, cervical cancer, chronic lymphocytic leukemia (CLL), chronic myelogenous leukemia (CML), colorectal cancer (CRC), endometrial cancer, esophageal cancer, eye cancer, gallbladder cancer, gastric (stomach) cancer, gastrointestinal stromal tumor (GIST), glioblastoma multiforme (GBM), hairy cell leukemia, head and neck cancer, heart cancer, hepatocellular (liver) carcinoma (HCC), Hodgkin's lymphoma and non-Hodgkin's lymphomas, Kaposi sarcoma, laryngeal cancer, mastocytosis, melanoma, myelofibrosis, myelodysplastic syndrome (MDS), multiple myeloma, non-small-cell lung carcinoma (NSCLC), lung cancer (small cell), melanoma, nasopharyngeal carcinoma, neuroendocrine tumors, neuroblastoma, oral cancer, oropharyngeal cancer, ovarian cancer, pancreatic cancer, paranasal sinus and nasal cavity cancer, parathyroid cancer, penile cancer, pharyngeal cancer, pituitary adenoma, prostate cancer, rectal cancer, renal cell (kidney) carcinoma (RCC), salivary gland cancer, skin cancer (nonmelanoma), small intestine cancer, small lymphocytic lymphoma (SSL), soft tissue sarcoma, squamous-cell carcinoma, T cell lymphoma, testicular cancer, throat cancer, thyroid cancer, triple negative breast cancer, urethral cancer, and uterine cancer.

164. The method according to claim 147, comprising administering gemcitabine and masitinib mesilate to the patient.

165. The method according to claim 147, comprising administering gemcitabine and masitinib mesilate to the patient, for the treatment of advanced or metastatic pancreatic cancer, breast cancer that has metastasized, advanced or metastatic non-small cell lung cancer, advanced or metastatic ovarian cancer, biliary tract cancer, bladder cancer, cervical cancer or malignant mesothelioma.

166. A method for treating cancer in a human patient with an under-expression, down-regulation, or decreased activity of dCK, wherein said method comprises administering to the human patient at least one small molecule inhibitor/activator or a pharmaceutically acceptable salt or solvate thereof in combination with at least one anticancer drug.

167. The method according to claim 166, comprising administering to the patient masitinib mesilate and gemcitabine.
